(19) United States
(12) Patent Application Publication
(10) Pub. No.: US 2026/0209316 A1
(43) Pub. Date: Jul. 23, 2026

(54) EBOLAVIRUS (SUDAN AND ZAIRE) ANTIBODIES FROM NON-HUMAN PRIMATES AND HUMAN VACCINEES

(71) Applicants: The U.S.A., as represented by the Secretary, Department of Health and Human Services, Bethesda, MD (US); Ridgeback Biotherapeutics, Miami, FL (US)

(72) Inventors: Nancy J. Sullivan, Brookline, MA (US); John Misasi, Dedham, MA (US); Thomas Niezold, Cambridge (GB); Bingchun Zhao, Bethesda, MD (US); Kendra Leigh, Germantown, MD (US); Ruth Hunegnaw, Bethesda, MD (US); Amarendra Pegu, Rockville, MD (US); Richard Koup, Washington, DC (US); Jean Christophe Sabue Mulangu, Frederick, MD (US)

(73) Assignees: The U.S.A., as represented by the Secretary, Department of Health and Human Services, Bethesda, MD (US); Ridgeback Biotherapeutics, Miami, FL (US)

(21) Appl. No.: 19/141,976

(22) PCT Filed: Dec. 22, 2023
(86) PCT No.: PCT/US2023/085745

§ 371 Date: Jun. 20, 2025

Related U.S. Application Data
(60) Provisional application No. 63/476,889, filed on Dec. 22, 2022.

(51) Int. Cl. C07K 16/10; A61K 39/00; A61P 31/14; G01N 33/569

(52) U.S. Cl. CPC C07K 16/10; A61P 31/14; G01N 33/56983; A61K 2039/505; A61K 2039/545; C07K 2317/31; C07K 2317/565; C07K 2317/76; G01N 2333/08; G01N 2800/26

(57) ABSTRACT

Monoclonal antibodies and bispecific monoclonal antibodies that bind Sudan virus (SUDV) and/or Ebola virus (EBOV) glycoprotein (GP) with nanomolar affinity are described. The monoclonal antibodies were developed by single-cell sorting of B cells obtained from non-human primate and human subjects previously immunized with Ebola (Zaire) virus (EBOV) and Sudan virus (SUDV) glycoprotein. Use of the monoclonal antibodies and bispecific antibodies for treating, inhibiting, and detecting infection by SUDV and EBOV is also described.

### CROSS REFERENCE TO RELATED APPLICATIONS

This application claims the benefit of U.S. Provisional Application No. 63/476,889, filed Dec. 22, 2022, which is herein incorporated by reference in its entirety.

### FIELD

This disclosure concerns monoclonal antibodies isolated by single-cell sorting of B cells obtained from non-human primate and human subjects previously immunized with Ebola (Zaire) virus (EBOV) and Sudan virus (SUDV) glycoprotein, and use of the antibodies for the treatment and prophylaxis against SUDV and EBOV infection.

### PARTIES TO A JOINT RESEARCH AGREEMENT

This invention was made under Cooperative Research and Development Agreement (CRADA) No. 2019-0971 between the National Institutes of Health National Institute of Allergy and Infectious Disease and Ridgeback Biotherapeutics.

### INCORPORATION OF ELECTRONIC SEQUENCE LISTING

The electronic sequence listing, submitted herewith as an XML file named 4239-109151-03.xml (176,097 bytes), created on Dec. 8, 2023, is herein incorporated by reference in its entirety.

### BACKGROUND

In 2013, the International Committee on the Taxonomy of Viruses (ICTV) Filoviridae Study Group and other experts published an updated taxonomy for filoviruses. The genus Ebolavirus is one of three genera in the family Filoviridae, which along with the genera Marburgvirus and Cuevavirus, are known to induce viral hemorrhagic fever. Six distinct species included in the genus Ebolavirus are Bundibugyo virus (BDBV), Reston virus (RESTV), Sudan virus (SUDV), Tal Forest virus (TAFV), Bombali virus (BOMV) and Ebola (also known as Zaire) virus (EBOV).

Ebolaviruses are large, negative-strand RNA viruses composed of 7 genes encoding viral proteins, including a single glycoprotein (GP). These viruses are responsible for causing Ebola virus disease (EVD), formerly known as Ebola hemorrhagic fever (EHF), in humans. In particular, BDBV, EBOV and SUDV have been associated with large outbreaks of EVD in Africa and reported case fatality rates of up to 90%. Transmission of Ebolavirus to humans is not yet fully understood, but is likely due to incidental exposure to infected animals. EVD spreads through human-to-human transmission, with infection resulting from direct contact with blood, secretions, organs or other bodily fluids of infected people, and indirect contact with environments contaminated by such fluids.

EVD has an incubation period of 2 to 21 days (7 days on average, depending on the strain) followed by a rapid onset of non-specific symptoms such as fever, extreme fatigue, gastrointestinal complaints, abdominal pain, anorexia, headache, myalgias and/or arthralgias. These initial symptoms last for about 2 to 7 days after which more severe symptoms related to hemorrhagic fever occur, including hemorrhagic rash, epistaxis, mucosal bleeding, hematuria, hemoptysis, hematemesis, melena, conjunctival hemorrhage, tachypnea, confusion, somnolence, and hearing loss. Laboratory findings include low white blood cell and platelet counts and elevated liver enzymes. In general, the symptoms last for about 7 to 14 days after which recovery may occur. Death can occur 6 to 16 days after the onset of symptoms. People are infectious as long as their blood and secretions contain the virus; the virus was isolated from semen 61 days after onset of illness in a man who was infected in a laboratory.

A very limited number of clinically approved therapeutics exist for the treatment of EVD. Thus, a need remains for the identification of effective therapeutic agents that prevent or treat infection by members of the Ebolavirus genus, particularly SUDV and EBOV.

### SUMMARY

Described herein are monoclonal antibodies and antigen binding fragments thereof that bind Sudan virus (SUDV) and/or Ebola virus (EBOV) glycoprotein (GP) with high (nanomolar) affinity. The disclosed monoclonal antibodies were isolated through nested PCR of the heavy and light chain immunoglobulin genes from GP-positive, single cell-sorted B cells from peripheral blood mononuclear cell (PBMC) samples of human and non-human primate (NHP) subjects previously immunized with Ebola (Zaire) virus (EBOV) and/or Sudan virus (SUDV) glycoprotein. The monoclonal antibodies and compositions thereof can be used for treating, inhibiting, and detecting infection by Ebolaviruses, such as SUDV and EBOV.

Provided herein are monoclonal antibodies that specifically bind SUDV and/or EBOV GP. The GP-specific monoclonal antibodies include the complementarity determining region (CDR) sequences (or the complete variable domains) of any one of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L and 396L antibodies.

Also provided herein are bispecific monoclonal antibodies that include a GP-specific monoclonal antibody disclosed herein. The bispecific monoclonal antibodies include a first antigen binding portion and a second antigen binding portion, wherein at least one of the first antigen binding portion and the second antigen binding portion includes the CDR sequences (or the complete variable domains) of any one of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L and 396L antibodies. In some aspects, the first antigen binding portion and the second antigen binding portion each individually include the CDR sequences (or the complete variable domains) of any one of antibodies disclosed herein. In other aspects, the first antigen binding portion includes the CDR sequences (or complete variable domains) of any one of the antibodies disclosed herein, and the second antigen binding portion includes the CDR sequences (or complete variable domains) of a different Ebolavirus-specific monoclonal antibody.

Also provided herein are antigen binding fragments of the disclosed monoclonal antibodies.

Isolated nucleic acid molecules and vectors encoding the monoclonal antibodies and bispecific monoclonal antibodies disclosed herein are also provided. Further provided are isolated cells that include a nucleic acid molecule or vector disclosed herein.

Also provided herein are compositions that include a pharmaceutically acceptable carrier and a monoclonal antibody, bispecific monoclonal antibody, nucleic acid molecule or vector disclosed herein.

Further provided are methods of detecting SUDV or EBOV GP in a sample by contacting the sample with a disclosed monoclonal antibody or bispecific monoclonal antibody under conditions sufficient to form an immune complex, and detecting the presence of the immune complex in the sample. Similarly, provided are methods of diagnosing an Ebolavirus infection (such as a SUDV or EBOV infection) in a subject by contacting a biological sample from the subject with a disclosed monoclonal antibody or bispecific monoclonal antibody under conditions sufficient to form an immune complex, and detecting the presence of the immune complex in the sample.

Also provided are methods of treating or inhibiting an Ebolavirus infection (such as a SUDV or EBOV infection) in a subject by administering to the subject a therapeutically effective amount of a monoclonal antibody, bispecific monoclonal antibody, nucleic acid molecule, vector, or composition disclosed herein. The methods can include post-infection treatment, post-exposure prophylaxis or pre-exposure prophylaxis. In some aspects, the method includes administering multiple (e.g., at least two, at least three, at least four, or at least 5) different monoclonal antibodies and/or bispecific antibodies disclosed herein to the subject.

The foregoing and other features of this disclosure will become more apparent from the following detailed description of several aspects which proceeds with reference to the accompanying figures.

### BRIEF DESCRIPTION OF THE DRAWINGS

FIGS. 1A-1D: Binding of 316L and 380L to Ebola GP measured by ELISA. Shown is antibody binding to full-length Sudan GP (GP_{FL}, FIG. 1A), mucin domain-deleted Sudan GP (GP_{dMuc}, FIG. 1B), Zaire GPF (FIG. 1C) and Zaire GP_{dMuc} (FIG. 1D). HIV-specific antibody VRC01 was included as a negative control, while antibodies 16F6 and mAb114 were included as positive controls in ELISA binding assays against Sudan GP and Zaire GP, respectively.

FIGS. 2A-2B: In vitro neutralization of SUDV GP- and EBOV GP-pseudotyped lentiviral vectors by 316L and 380L. (FIG. 2A) Microneutralization assay against SUDV GP-pseudotyped lentivirus. (FIG. 2B) Microneutralization assay against EBOV GP-pseudotyped lentivirus. VRC01 was included as a negative control. 16F6 and mAb114 were used as positive controls in the neutralization assays. Error bars represent the standard deviation of triplicate well values.

FIG. 3: Niemann-Pick disease, type C1-domain C (NPC1-dC) competition assay measured by biolayer interferometry (BLI). Shown is the percent inhibition of NPC1-dC receptor binding to thermolysin-cleaved SUDV GP (GP(S)THL) by antibodies 316L and 380L. Negative control 16F6, which binds to the SUDV GP base region, showed no blocking of NPC1-dC binding to GP(S)THL. NPC1-dC self-competition and mAb114, included as positive controls, showed 79% and 86% blocking of NPC1-dC binding to GP(S)THL, respectively. 316L and 380L each blocked 71% of NPC1-dC binding.

FIG. 4: Immunoprecipitation assay to detect direct interaction of antibody (316L, 380L) with thermolysin cleaved SUDV GP (GP(S)THL). Incubation of GP(S)THL with no mAb or an irrelevant mAb were included as negative controls. SUDV GP base binder 16F6 was used as a positive control. The results show that like 16F6, 316L and 380L were immunoprecipitated with GP(S)THL, indicating they directly interact with GP(S)THL.

FIG. 5: Competition group of antibodies 316L and 380L determined by BLI. The order of addition to the biosensors was: mucin domain-deleted GP (antigen), competitor mAb, and analyte mAb. 15H10 binds to the low base region of GP near the membrane-proximal external region/heptad repeat 2 (MPER/HR2) site. KZ52 binds to one protomer of the GP trimer. mAb114 binds the GP trimer from the top. 13C6 binds to the medial portion of the glycan cap, while mAb166 binds to the lateral GP. 16F6 binds to base region of GP.

FIG. 6: Class average images from negative-stain transmission electron micrographs of Fabs complexed with mucin domain-deleted (dMuc) GP. GP is indicated by dotted lines, and Fab is indicated by red arrows.

FIG. 7: Schematic of SUDV challenge and monoclonal antibody (mAb) dosing timeline for in vivo efficacy studies.

FIG. 8: Kaplan-Meier curves showing survival of animals treated with 316L or 380L and challenged with SUDV. Data are combined from two independent experiments. Antibodies 316L and 380L were administered three times at a dose of 50 mg/kg/dose.

FIG. 9: Kaplan-Meier curves showing survival in an in vivo efficacy study. Animals were treated with a 1:1 mixture of the 3106L and 380L antibodies at a dose of 50 mg/kg. Treated animals were compared to a contemporaneous untreated control group and a historical untreated control group.

FIGS. 10A-10B: Binding of antibodies 291S and 545S to Sudan GP determined by ELISA assay. (FIG. 10A) Binding of 291S and 545S to SUDV GPL. (FIG. 10B) Binding of 291S and 545S to SUDV GP_{dMuc}. HIV-specific antibody VRC01 was included as a negative control.

FIG. 11: In vitro neutralization of SUDV GP-pseudotyped lentiviral vectors by 291S and 545S. VRC01 was included as a negative control. Error bars represent the standard deviation of triplicate well values.

FIG. 12: NPC1-dC competition assay measured by BLI. Shown is the percent inhibition of NPC1-dC receptor binding to thermolysin-cleaved SUDV GP (GP(S)THL) by antibodies 291S and 545S. VRC01 and 16F6 were included as negative controls. NPC1-dC (dC) self-competition, included as a positive control, showed 72.0% blocking of NPC1-dC binding to GP(S)THL. 291S and 545S blocked 63.5% and 74.9% of NPC1-dC binding, respectively.

FIG. 13: Competition group of antibody 316L as determined by BLI. The order of addition to the biosensors was: GP_{dMuc} (antigen), competitor mAb, analyte mAb. 16F6 binds to the base region of GP. 15H10 binds to the low base region of GP near the MPER/HR2 site. mAb166 binds to glycan cap of the GP trimer. 316L binds the GP trimer from the top. Results of the competition study demonstrate that antibody 316L is in the same competition group as antibodies 291S and 545S.

FIG. 14: Class average images from negative-stain transmission electron micrographs of Fabs complexed with mucin domain-deleted GP. GP is indicated by dotted lines, and Fab is indicated by red arrows.

FIG. 15: Kaplan-Meier curves showing survival in an in vivo efficacy study in cynomolgus macaques. Animals were treated with a 1:1 mixture of the 545S and 523S antibodies at a dose of 50 mg/kg. Twenty-four hours after lethal challenge (100 PFU) with Sudan Gulu strain, animals were administered the antibody cocktail by three IV injections at 24-hour intervals. Treated animals were compared to a contemporaneous untreated control animal and a historical untreated control group.

FIGS. 16A-16B: Binding of antibodies to Sudan GP determined by ELISA assay. Binding of antibodies 523S, 573S, 541S, 294S, 241S, 345S, 233S and 503S to SUDV GP_{FL} (FIG. 16A) and SUDV GP_{dMuc} (FIG. 16B) is shown. VRC01 was included as a negative control.

FIG. 17: In vitro neutralization of SUDV GP-pseudotyped lentiviral vectors by antibodies 523S, 573S, 541S, 294S, 241S, 345S, 233S and 503S. VRC01 was included as a negative control. Error bars represent the standard deviation of triplicate well values.

FIG. 18: Competition groups of 523S, 573S, 541S, 294S, 241S, 345S, 233S and 503S as determined by BLL The order of addition to the biosensors was: GP_{dMuc} (antigen), competitor mAb, analyte mAb. The results show that 523S, 573S and 541S are in the same competition group as 16F6, which binds at the base region of GP.

FIG. 19: Class average images from negative-stain transmission electron micrographs of Fabs complexed with mucin domain-deleted GP. 2D class averages of Fab100 were generated from complex with mucin domain-deleted Zaire GP. mAb100 is a GP base binder, and binds to interprotomer of GP. ma-C10 binds to the MPER/HR2 region of GP. GP is indicated by dotted lines, and Fab is indicated by red arrows.

FIGS. 20A-20B: Binding of antibodies to Sudan GP determined by ELISA. Binding of antibodies 203S, 315S, 586S, 377S, 528S, 246S, 338S, 335S, 285S, 382S, 365S, 405S, 102S and 578S to SUDV GP_{FL} (FIG. 20A) and SUDV GP_{dMuc} (FIG. 20B) is shown. VRC01 was included as a negative control.

FIG. 21: In vitro neutralization of SUDV GP-pseudotyped lentiviral vectors by antibodies 203S, 315S, 586S, 377S, 528S, 246S, 338S, 335S, 285S, 382S, 365S, 405S, 102S and 578S. VRC01 was included as a negative control. Error bars represent the standard deviation of triplicate well values.

FIG. 22: Competition groups of antibodies 203S, 315S, 586S, 377S, 528S, 248S, 338S, 335S, 285S, 382S, 365S, 405S, 102S and 578S as determined by BLI. The order of addition to the biosensors was: GP_{dMuc} (antigen), competitor mAb, analyte mAb.

FIGS. 23A-23B: Class average images from negative-stain transmission electron micrographs of Fabs complexed with GP_{dMuc}. 2D class averages of Fab166 were generated from complex with mucin domain-deleted Zaire GP. mAb166 is a GP glycan cap binder. GP is indicated by dotted lines, and Fab is indicated by red arrows.

FIGS. 24A-24D: Binding of antibodies to Ebola GP determined by ELISA. Binding of antibodies 191L, 206L, 231L, 232L, 310L, 314L, 315L and 396L to SUDV GP_{FL}, (FIG. 24A), SUDV GP_{dMuc} (FIG. 24B), Zaire GP_{FL}, (FIG. 24C) and Zaire GP_{dMuc} (FIG. 24D) is shown. VRC01 was included as a negative control. 16F6 and mAb114 were included as positive controls in ELISA binding assays against Sudan GP and Zaire GP, respectively.

FIG. 25: In vitro neutralization of SUDV GP- and EBOV GP-pseudotyped lentiviral vectors by 191L, 206L, 231L, 232L, 310L, 315L. VRC01 was included as a negative control. 16F6 and mAb114 were included as positive controls. Error bars represent the standard deviation of triplicate well values.

FIG. 26: Schematic of bispecific antibodies BiSp201LS and BiSp202LS.

FIG. 27: SDS-PAGE analysis of BiSp201LS and BiSp202LS purity and association.

FIG. 28: Analytic HPLC trace of purified BiSp201LS (top) and BiSp202LS (bottom).

FIG. 29: Neutralization of lentiviruses pseudotyped with Sudan GP by BiSp201LS or a mixture of antibodies 523S and 545S. VRC01 was included as a negative control.

FIG. 30: Kaplan-Meier curve showing percent survival of animals treated with a cocktail of antibodies 545S and 523S after challenge with SUDV. Cynomolgus macaques (n=3) were challenged with 1000 PFU SUDV Gulu on day 0 (DO) and administered IV injections of a 1:1 cocktail of 545S+523S (50 mg/kg total) on D1, D2 and D3 (represented by the vertical dashed lines). A study control animal (n=1) was challenged with SUDV but did not receive treatment. The treated and control animals were compared to historical untreated control animals (n=4).

FIGS. 31A-31B: Kaplan-Meier curves showing percent survival of animals treated with a single dose of a cocktail of antibodies 545S and 523S after challenge with SUDV. (FIG. 31A) Cynomolgus macaques (n=3) were challenged with 1000 PFU SUDV Gulu on DO and then infused with a 1:1 cocktail of 545S+523S (50 mg/kg total) on D4. Study control animals (n=3) were challenged with SUDV but did not receive treatment. The treated and control animals were compared to historical untreated controls (n=5). (FIG. 31B) Cynomolgus macaques (n=3) were challenged with 1000 PFU SUDV Gulu on DO and then infused with a 1:1 cocktail of 545S+523S (50 mg/kg total) on D5. A study control animal (n=1) was challenged with SUDV but did not receive treatment. The treated and control animals were compared to historical untreated control animals (n=12).

FIG. 32: Kaplan-Meier curve showing percent survival of animals treated with lower doses of the 545S+523S antibody cocktail. Cynomolgus macaques were challenged with 1000 PFU SUDV Gulu on DO and then infused with a 1:1 cocktail of 545S+523S at a dose of 20 mg/kg total (n=3) or 5 mg/kg (n=3) on D5. A study control animal (n=1) was challenged with SUDV but did not receive treatment. The treated and control animals were compared to historical untreated controls (n=13).

FIG. 33: Kaplan-Meier curve showing percent survival of animals treated with a cocktail of antibodies 545S and 523S prior to challenge with SUDV. Three days prior to challenge with 1000 PFU SUDV Gulu, cynomolgus macaques were infused with a 1:1 cocktail of 545S+523S (50 mg/kg total) (n=3). A study control animal (n=1) was challenged with SUDV but did not receive treatment. The treated and control animals were compared to historical untreated control animals (n=12).

FIGS. 34A-34B: Kaplan-Meier curves showing percent survival of animals treated with BiSp201LS after challenge with SUDV. (FIG. 34A) Cynomolgus macaques (n=3) were challenged with 1000 PFU SUDV Gulu on DO and then infused with BiSp201LS at a dose of 50 mg/kg on D4. A study control animal (n=1) was challenged with SUDV but did not receive treatment. The treated and control animals were compared to historical untreated control animals (n=12). (FIG. 34B) Cynomolgus macaques (n=3) were challenged with 1000 PFU SUDV Gulu on DO and then infused with BiSp201LS at a dose of 50 mg/kg on D5. Study control animals (n=2) were challenged with SUDV but did not receive treatment. The treated and control animals were compared to historical untreated control animals (n=12).

FIG. 35: Kaplan-Meier curve showing percent survival of animals treated with BiSp201LS prior to challenge with SUDV. Cynomolgus macaques (n=3) were infused with the bispecific antibody BiSp201LS at a dose of 50 mg/kg three days prior to challenge with 1000 PFU of SUDV Gulu on DO. A study control animal (n=1) was challenged with SUDV but did not receive treatment. The treated and control animals were compared to historical untreated control animals (n=12).

### SEQUENCE LISTING

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing: SEQ ID NOs: 1 and 2 are the nucleotide and amino acid sequences of the VH domain of antibody 316L.

SEQ ID NOs: 3 and 4 are the nucleotide and amino acid sequences of the VL domain of antibody 316L.

SEQ ID NOs: 5 and 6 are the nucleotide and amino acid sequences of the VH domain of antibody 380L.

SEQ ID NOs: 7 and 8 are the nucleotide and amino acid sequences of the VL domain of antibody 380L.

SEQ ID NOs: 9 and 10 are the nucleotide and amino acid sequences of the VH domain of antibody 291S.

SEQ ID NOs: 11 and 12 are the nucleotide and amino acid sequences of the VL domain of antibody 291S.

SEQ ID NOs: 13 and 14 are the nucleotide and amino acid sequences of the VH domain of antibody 545S.

SEQ ID NOs: 15 and 16 are the nucleotide and amino acid sequences of the VL domain of antibody 545S.

SEQ ID NOs: 17 and 18 are the nucleotide and amino acid sequences of the VH domain of antibody 523S.

SEQ ID NOs: 19 and 20 are the nucleotide and amino acid sequences of the VL domain of antibody 523S.

SEQ ID NOs: 21 and 22 are the nucleotide and amino acid sequences of the VH domain of antibody 573S.

SEQ ID NOs: 23 and 24 are the nucleotide and amino acid sequences of the VL domain of antibody 573S.

SEQ ID NOs: 25 and 26 are the nucleotide and amino acid sequences of the VH domain of antibody 541S.

SEQ ID NOs: 27 and 28 are the nucleotide and amino acid sequences of the VL domain of antibody 514S.

SEQ ID NOs: 29 and 30 are the nucleotide and amino acid sequences of the VH domain of antibody 294S.

SEQ ID NOs: 31 and 32 are the nucleotide and amino acid sequences of the VL domain of antibody 294S.

SEQ ID NOs: 33 and 34 are the nucleotide and amino acid sequences of the VH domain of antibody 241S.

SEQ ID NOs: 35 and 36 are the nucleotide and amino acid sequences of the VL domain of antibody 241S.

SEQ ID NOs: 37 and 38 are the nucleotide and amino acid sequences of the VH domain of antibody 354S.

SEQ ID NOs: 39 and 40 are the nucleotide and amino acid sequences of the VL domain of antibody 354S.

SEQ ID NOs: 41 and 42 are the nucleotide and amino acid sequences of the VH domain of antibody 233S.

SEQ ID NOs: 43 and 44 are the nucleotide and amino acid sequences of the VL domain of antibody 233S.

SEQ ID NOs: 45 and 46 are the nucleotide and amino acid sequences of the VH domain of antibody 503S.

SEQ ID NOs: 47 and 48 are the nucleotide and amino acid sequences of the VL domain of antibody 503S.

SEQ ID NOs: 49 and 50 are the nucleotide and amino acid sequences of the VH domain of antibody 203S.

SEQ ID NOs: 51 and 52 are the nucleotide and amino acid sequences of the VL domain of antibody 203S.

SEQ ID NOs: 53 and 54 are the nucleotide and amino acid sequences of the VH domain of antibody 315S.

SEQ ID NOs: 55 and 56 are the nucleotide and amino acid sequences of the VL domain of antibody 315S.

SEQ ID NOs: 57 and 58 are the nucleotide and amino acid sequences of the VH domain of antibody 586S.

SEQ ID NOs: 59 and 60 are the nucleotide and amino acid sequences of the VL domain of antibody 586S.

SEQ ID NOs: 61 and 62 are the nucleotide and amino acid sequences of the VH domain of antibody 377S.

SEQ ID NOs: 63 and 64 are the nucleotide and amino acid sequences of the VL domain of antibody 377S.

SEQ ID NOs: 65 and 66 are the nucleotide and amino acid sequences of the VH domain of antibody 528S.

SEQ ID NOs: 67 and 68 are the nucleotide and amino acid sequences of the VL domain of antibody 528S.

SEQ ID NOs: 69 and 70 are the nucleotide and amino acid sequences of the VH domain of antibody 246S.

SEQ ID NOs: 71 and 72 are the nucleotide and amino acid sequences of the VL domain of antibody 246S.

SEQ ID NOs: 73 and 74 are the nucleotide and amino acid sequences of the VH domain of antibody 338S.

SEQ ID NOs: 75 and 76 are the nucleotide and amino acid sequences of the VL domain of antibody 338S.

SEQ ID NOs: 77 and 78 are the nucleotide and amino acid sequences of the VH domain of antibody 335S.

SEQ ID NOs: 79 and 80 are the nucleotide and amino acid sequences of the VL domain of antibody 335S.

SEQ ID NOs: 81 and 82 are the nucleotide and amino acid sequences of the VH domain of antibody 285S.

SEQ ID NOs: 83 and 84 are the nucleotide and amino acid sequences of the VL domain of antibody 285S.

SEQ ID NOs: 85 and 86 are the nucleotide and amino acid sequences of the VH domain of antibody 382S.

SEQ ID NOs: 87 and 88 are the nucleotide and amino acid sequences of the VL domain of antibody 382S.

SEQ ID NOs: 89 and 90 are the nucleotide and amino acid sequences of the VH domain of antibody 365S.

SEQ ID NOs: 91 and 92 are the nucleotide and amino acid sequences of the VL domain of antibody 365S.

SEQ ID NOs: 93 and 94 are the nucleotide and amino acid sequences of the VH domain of antibody 405S.

SEQ ID NOs: 95 and 96 are the nucleotide and amino acid sequences of the VL domain of antibody 405S.

SEQ ID NOs: 97 and 98 are the nucleotide and amino acid sequences of the VH domain of antibody 102S.

SEQ ID NOs: 99 and 100 are the nucleotide and amino acid sequences of the VL domain of antibody 102S.

SEQ ID NOs: 101 and 102 are the nucleotide and amino acid sequences of the VH domain of antibody 578S.

SEQ ID NOs: 103 and 104 are the nucleotide and amino acid sequences of the VL domain of antibody 578S.

SEQ ID NOs: 105 and 106 are the nucleotide and amino acid sequences of the VH domain of antibody 191L.

SEQ ID NOs: 107 and 108 are the nucleotide and amino acid sequences of the VL domain of antibody 191L.

SEQ ID NOs: 109 and 110 are the nucleotide and amino acid sequences of the VH domain of antibody 206L.

SEQ ID NOs: 111 and 112 are the nucleotide and amino acid sequences of the VL domain of antibody 206L.

SEQ ID NOs: 113 and 114 are the nucleotide and amino acid sequences of the VH domain of antibody 231L.

SEQ ID NOs: 115 and 116 are the nucleotide and amino acid sequences of the VL domain of antibody 231L.

SEQ ID NOs: 117 and 118 are the nucleotide and amino acid sequences of the VH domain of antibody 232L.

SEQ ID NOs: 119 and 120 are the nucleotide and amino acid sequences of the VL domain of antibody 232L.

SEQ ID NOs: 121 and 122 are the nucleotide and amino acid sequences of the VH domain of antibody 310L.

SEQ ID NOs: 123 and 124 are the nucleotide and amino acid sequences of the VL domain of antibody 310L.

SEQ ID NOs: 125 and 126 are the nucleotide and amino acid sequences of the VH domain of antibody 314L.

SEQ ID NOs: 127 and 128 are the nucleotide and amino acid sequences of the VL domain of antibody 314L.

SEQ ID NOs: 129 and 130 are the nucleotide and amino acid sequences of the VH domain of antibody 315L.

SEQ ID NOs: 131 and 132 are the nucleotide and amino acid sequences of the VL domain of antibody 315L.

SEQ ID NOs: 133 and 134 are the nucleotide and amino acid sequences of the VH domain of antibody 396L.

SEQ ID NOs: 135 and 136 are the nucleotide and amino acid sequences of the VL domain of antibody 396L.

SEQ ID NO: 137 is the amino acid sequence of the 316L heavy chain.

SEQ ID NO: 138 is the amino acid sequence of the 316L light chain.

SEQ ID NO: 139 is the amino acid sequence of the 545S heavy chain.

SEQ ID NO: 140 is the amino acid sequence of the 545S light chain.

SEQ ID NO: 141 is the amino acid sequence of the 523S heavy chain.

SEQ ID NO: 142 is the amino acid sequence of the 523S light chain.

SEQ ID NO: 143 is the amino acid sequence of the 294S heavy chain.

SEQ ID NO: 144 is the amino acid sequence of the 294S light chain.

SEQ ID NOs: 145 and 146 are exemplary leader sequences.

SEQ ID NO: 147 is the amino acid sequence of a macaque heavy chain (IgG1) constant region.

SEQ ID NO: 148 is the amino acid sequence of a macaque lambda light chain constant region.

SEQ ID NO: 149 is the amino acid sequence of a human heavy chain (IgG1) constant region.

SEQ ID NO: 150 is the amino acid sequence of a human lambda light chain constant region.

SEQ ID NO: 151 is the amino acid sequence of a human kappa light chain constant region.

SEQ ID NO: 152 is the amino acid sequence of the unswapped Fv523 heavy chain.

SEQ ID NO: 153 is the amino acid sequence of the unswapped Fv523 lambda light chain.

SEQ ID NO: 154 is the amino acid sequence of the swapped Fv545 heavy chain.

SEQ ID NO: 155 is the amino acid sequence of the swapped Fv545 lambda light chain.

SEQ ID NO: 156 is the amino acid sequence of the unswapped Fv545 heavy chain.

SEQ ID NO: 157 is the amino acid sequence of the unswapped Fv545 lambda light chain.

SEQ ID NO: 158 is the amino acid sequence of the swapped Fv523 heavy chain.

SEQ ID NO: 159 is the amino acid sequence of the swapped Fv523 lambda light chain.

### DETAILED DESCRIPTION

### I. Introduction

The 2014 outbreak of EVD in West Africa was first recognized in March 2014, and as of Apr. 13, 2016, the number of cases far exceeded the largest prior EVD outbreak with a combined total (suspected, probable, and laboratory-confirmed) 28,616 cases and 11,310 deaths, resulting in a case fatality rate of 39.5%. In 2017, there was a subsequent outbreak of Zaire ebolavirus (EBOV) with a total of 8 cases and 4 deaths in the Democratic Republic of Congo (DRC) (case fatality rate of 50%). The most recent outbreak of EBOV also occurred in DRC, which resulted in a total of 54 cases and 33 deaths (case fatality rate of 61%). The largest previous outbreak of EBOV EVD occurred in Uganda in 2000-2001, with 425 cases and 224 deaths (case fatality rate of 53%).

Sudan ebolavirus (SUDV) is the second most common cause of EVD. SUDV EVD first broke out in southern Sudan in 1976 at the same time the first EBOV EVD was occurring in the DRC. A subsequent outbreak of SUDV occurred in June-August 2012, with 24 confirmed cases and 17 deaths reported in Uganda. The fatality rate was as high as 71%. In October 2000, the third and largest outbreak of infection occurred in the Gulu district of Uganda. During the course of the outbreak (August 2000-January 2001), 1400 cases of SUDV infection were identified. Similar to what had been reported for earlier SUDV infection outbreaks, the case fatality rate was 53%, with a mean time to death of 8 days after onset of symptoms. SUDV was associated with outbreaks in 1976 and 1979 in the southern Sudan towns of Nzara and Maridi; in 2000 in Gulu, Uganda; and in 2004 in Yambio, Sudan. The average fatality rates for SUDV were 54% in 1976, 68% in 1979, 53% in 2000 and 2001, and 41% in 2004.

While prior outbreaks of EVD have been localized to regions of Africa, there is a potential threat of spread to other countries given the frequency of international travel. Viruses in the Filoviridae family are also categorized as potential threats for use as biological weapons due to ease of dissemination and transmission, and high levels of mortality. With the exception of the Zaire ebolavirus-specific antibody-based therapeutics EBANGA™ (formerly mAb114) and INMAZEB™ (a combination of three monoclonal antibodies), no effective therapies or FDA-licensed vaccines exist for any member of the Filoviridae family of viruses. Given the high mortality rate of Ebolavirus infections and the potential risk of their use as a biological weapon, a need exists for additional therapeutics for the treatment of infection by these viruses, particularly for SUDV infection.

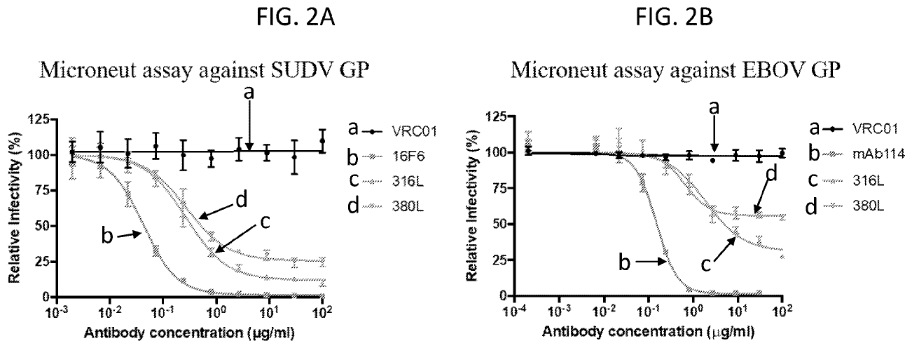

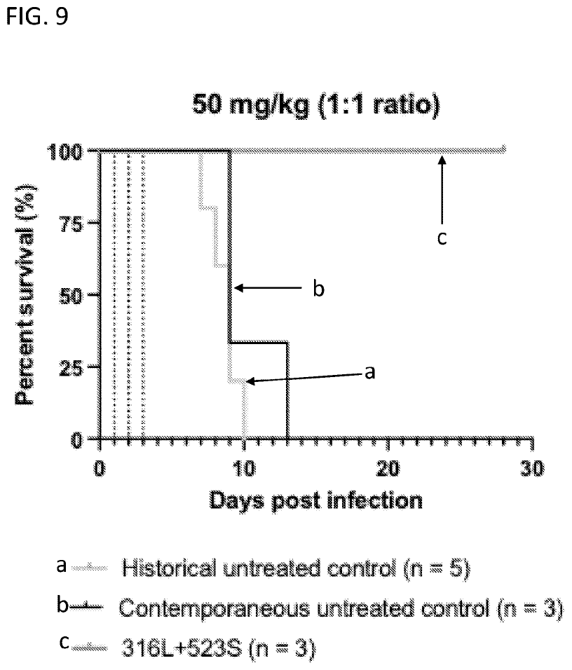

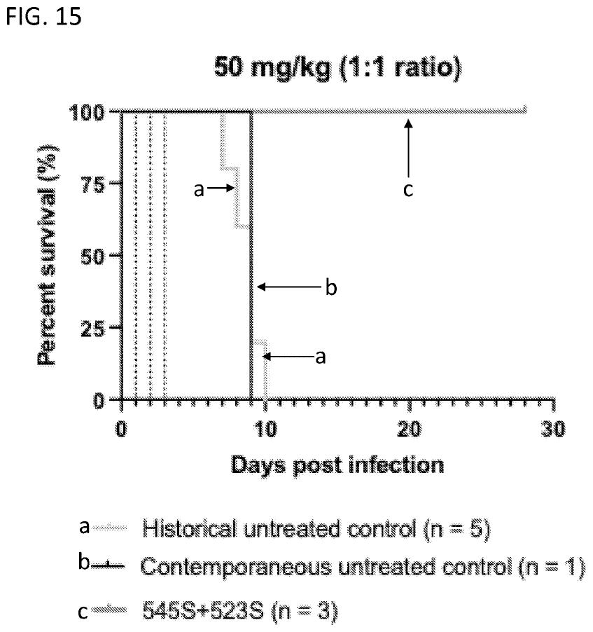

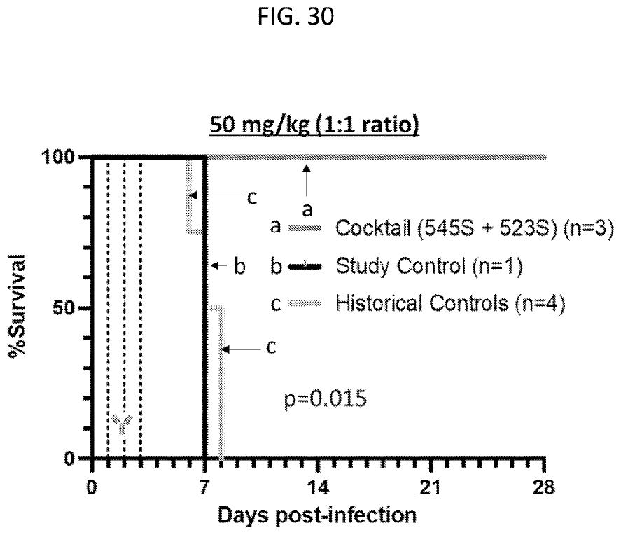

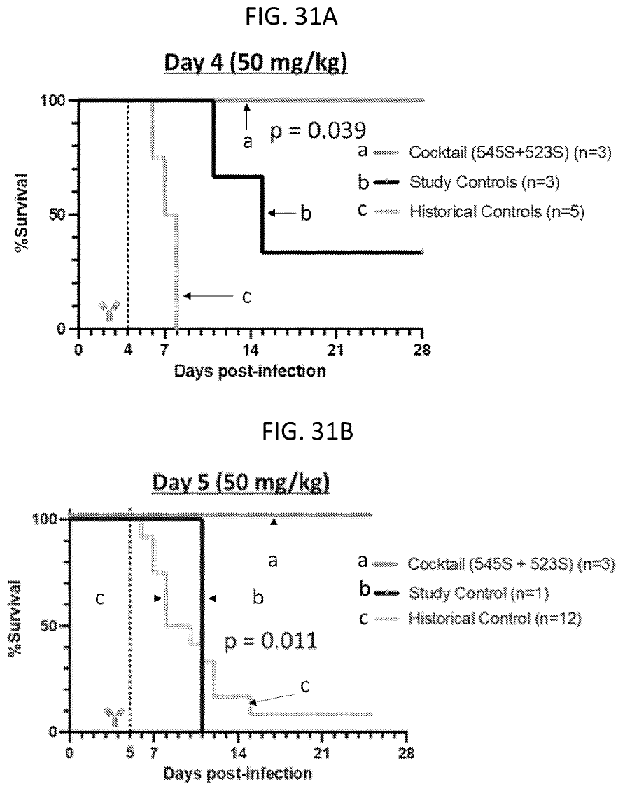

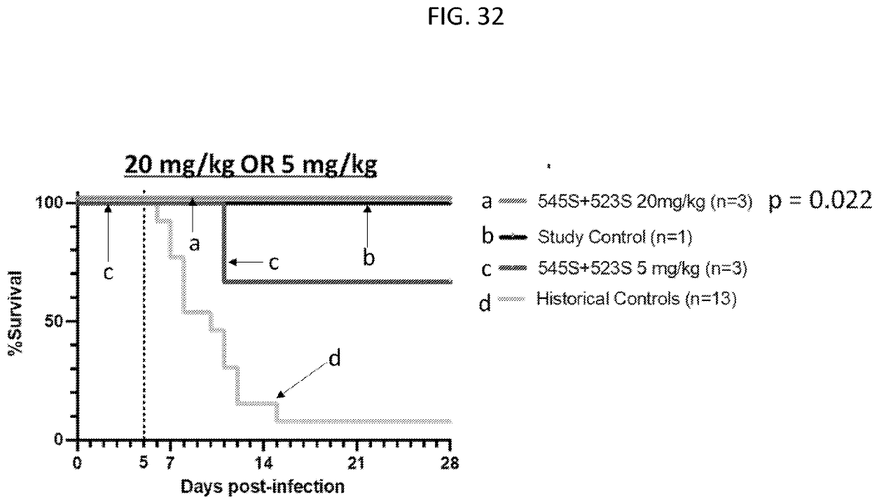

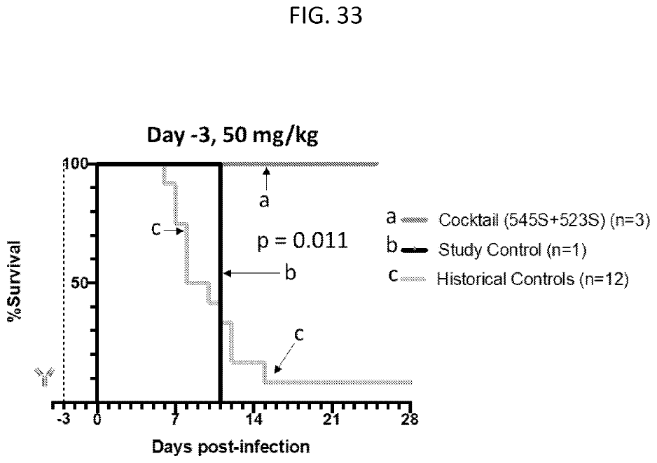

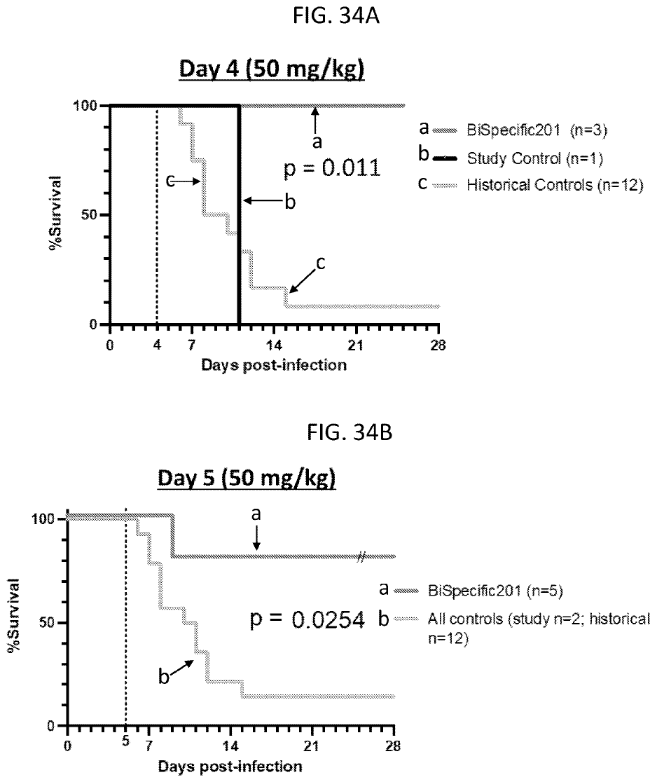

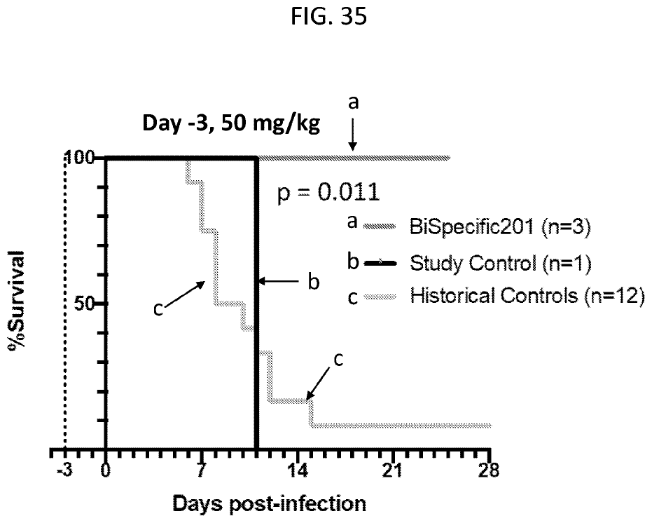

The monoclonal antibodies disclosed herein satisfy this unmet need. Several of the disclosed antibodies (e.g., 316L, 380L, 291S and 545S) bind an epitope of GP that is near the receptor binding site (RBS) for SUDV and EBOV. Antibodies that bind this epitope have rarely been reported for EBOV and until the present disclosure, have never been reported for SUDV. Other antibodies disclosed herein have epitopes in the MPER/HR2 region of GP (e.g., 294S, 241S, 354S, 233S and 503S), the base region of GP (e.g., 523S, 573S and 541S), or the glycan cap region of GP (e.g., 103S, 315S, 586S, 377S, 528S, 246S, 338S, 335S, 285S, 382S, 365S, 405S, 102S and 578S). The disclosed antibodies bind SUDV and/or EBOV GP with high affinity (in the nanomolar range; see Tables 3, 8, 12 and 15) and are capable of potently neutralizing infection by SUDV GP- and/or EBOV GP-pseudotyped lentiviruses (FIGS. 2A, 2B, 11, 17 and 21). Furthermore, studies disclosed herein demonstrate that 316L and 380L provide potent protection against SUDV (Gulu strain) challenges in a NHP model (FIG. 8). The antibody combinations of 316L+523S and 545S+523S also fully protected NHPs from lethal SUDV challenge (FIGS. 9 and 15). In addition, the antibody cocktail of 545S+523S was protective against lethal (1000 PFU) SUDV challenge when administered in a single dose (FIGS. 30-33) either before challenge (e.g., 3 days prior to challenge; FIG. 33), or after challenge (e.g., 4 or 5 days after challenge; FIGS. 31A-31B), including at relatively lower doses of 20 mg/kg or 5 mg/kg (FIG. 32). Furthermore, data disclosed herein demonstrates that a bispecific antibody having the antigen-binding portions of antibodies 545S and 523S (“BiSp201LS”) provides complete protection against SUDV challenge in NHPs when the bispecific antibody is administered in a single dose either before challenge (e.g., 3 days prior to challenge; FIG. 35) or after challenge (e.g., 4 or 5 days after challenge; FIGS. 34A-34B).

Taken together, these results demonstrate that the monoclonal antibodies, antibody cocktails and bispecific antibodies disclosed herein provide effective post-exposure and pre-exposure protection against SUDV using as little as one dose.

### II. Abbreviations

BDBV Bundibugyo virus
BLI biolayer interferometry
BOMV Bombali virus
EBOV Ebola virus
EVD Ebola virus disease
GP glycoprotein
GP(S)THL thermolysin-cleaved SUDV GP
HR2 heptad repeat 2
IC₅₀ inhibitory concentration 50
IV intravenous
mAb monoclonal antibody
MPER membrane-proximal external region
NHP non-human primate
NPC1-dC Niemann-Pick disease, type C1-domain C
OD optical density
PBMC peripheral blood mononuclear cells
PFU plaque forming units
RBS receptor binding site
RESTV Reston virus
SUDV Sudan virus
TAFV Tai Forest virus
VH variable heavy
VL variable light

### III. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, and intravenous), infusion, sublingual, rectal, transdermal (for example, topical), intranasal, vaginal, and inhalation routes.

Antibody and Antigen Binding Fragment: An immunoglobulin, antigen-binding fragment, or derivative thereof, that specifically binds and recognizes an analyte (antigen) such as Ebola virus GP. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity.

Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)₂; diabodies; linear antibodies; single-chain antibody molecules (e.g., scFv, V_{H}H); and multispecific antibodies formed from antibody fragments. Antibody fragments include antigen binding fragments either produced by the modification of whole antibodies or those synthesized de novo using recombinant DNA methodologies (see, e.g., Kontermann and Dfibel (Eds.), Antibody Engineering, Vols. 1-2, 2^{ND} ed., Springer-Verlag, 2010).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242(4877):423-426, 1988; Huston et al., Proc. Natl. Acad. Sci. U.S.A., 85(16):5879-5883, 1988; Ahmad et al., Clin. Dev. Immunol., 2012, doi:10.1155/2012/980250; Marbry and Snavely, IDrugs, 13(8):543-549, 2010). The intramolecular orientation of the V_{H} domain and the V_{L} domain in a scFv is typically not decisive for scFvs. Thus, scFvs with both possible arrangements (V_{H} domain-linker domain-V_{L} domain; V_{L} domain-linker domain-V_{H} domain) may be used.

In a dsFv, the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci. U.S.A., 90(14):6444-6448, 1993; Poljak et al., Structure, 2(12):1121-1123, 1994).

Antibodies also include genetically engineered forms such as chimeric antibodies (such as humanized murine or macaque antibodies) and heteroconjugate antibodies (such as bispecific antibodies).

Typically, a naturally occurring mammalian immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) that determine the functional activity of a mammalian antibody molecule: IgM, IgD, IgG, IgA and IgE.

Each heavy and light chain contain a constant region (or constant domain) and a variable region (or variable domain). In several aspects, the V_{H} and V_{L} combine to specifically bind the antigen. In additional aspects, only the V_{H} is required. For example, naturally occurring camelid antibodies consisting of a heavy chain only (V_{H}H) are functional and stable in the absence of light chain. Any of the disclosed antibodies can include a heterologous constant domain. For example, the antibody can include a constant domain that is different from a native constant domain, such as a constant domain including one or more modifications (such as the “LS” mutations) to increase half-life.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples, monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a non-human primate, mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one aspect, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody that includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some aspects, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} ed. New York: Cold Spring Harbor Laboratory Press, 2004; Lonberg, Nat. Biotechnol., 23(9): 1117-1125, 2005; Lonberg, Curr. Opin. Immunol. 20(4):450-459, 2008).

Binding affinity: Affinity of an antibody (or bispecific antibody) for an antigen. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using biolayer interferometry (BLI) technology, such as by using the Octet system (Creative Biolabs). In other aspects, Kd is measured using a surface plasmon resonance (SPR) assay, such as by using a BIACORES-2000 or a BIACORES-3000 (BIAcore, Inc., Piscataway, N.J.). In other aspects, antibody affinity is measured by flow cytometry. An antibody that “specifically binds” an antigen (such as SUDV and/or EBOV glycoprotein) is an antibody that binds the antigen with high affinity and does not significantly bind other unrelated antigens.

Biological sample: A sample obtained from a subject. Biological samples include all clinical samples useful for detection of disease or infection (for example, SUDV or EBOV infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having an EBOV or SUDV infection.

Bispecific antibody: A recombinant molecule composed of two different antigen binding portions that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain. In some aspects disclosed herein, the bispecific antibody is in the CrossMab format (Roche). Similarly, a multi-specific antibody is a recombinant protein that includes antigen-binding fragments of at least two different monoclonal antibodies, such as two, three or four different monoclonal antibodies.

Conditions sufficient to form an immune complex: Conditions that allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Harlow & Lane, Antibodies, A Laboratory Manual, 2^{nd} ed. Cold Spring Harbor Publications, New York (2013) for a description of immunoassay formats and conditions. The conditions employed in the methods are “physiological conditions” which include reference to conditions (such as temperature, osmolarity, pH) that are typical inside a living mammal or a mammalian cell. While it is recognized that some organs are subject to extreme conditions, the intra-organismal and intracellular environment normally lies around pH 7 (for example, from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, X-ray and affinity chromatography. Immunological binding properties of selected antibodies may be quantified using well-known methods.

Conjugate: A complex of two molecules linked together, for example, linked together by a covalent bond. In some aspects, an antibody or a bispecific antibody disclosed herein is linked to an effector molecule, such as covalently linked to an effector molecule, or to a detectable label. The linkage can be by chemical or recombinant means. In one aspect, the linkage is chemical, wherein a reaction between the antibody moiety and the effector molecule has produced a covalent bond formed between the two molecules to form one molecule. A peptide linker (short peptide sequence) can optionally be included between the antibody/bispecific antibody and the effector molecule.

Conservative amino acid substitution: “Conservative” amino acid substitutions are those substitutions that do not substantially affect a function of a protein, such as the ability of the protein to interact with a target protein.

In some aspects, a conservative amino acid substitution in a SUDV or EBOV GP-specific antibody is one that does not reduce binding of the antibody to GP by more than 10% (such as by more than 5%) compared to the GP binding of the corresponding antibody lacking the conservative amino acid substitution.

In some aspects, the SUDV or EBOV GP-specific antibody can include up to 1, 2, 3, 4, 5, 6, 7, 8, 9, or up to 10 conservative substitutions compared to a reference antibody and retain specific binding activity for GP, and/or retain SUDV/EBOV neutralization activity.

Typically, individual substitutions, deletions or additions which alter, add or delete a single amino acid or a small percentage of amino acids (for instance less than 5%, in some aspects less than 1%) in an encoded sequence are conservative variations where the alterations result in the substitution of an amino acid with a chemically similar amino acid. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some aspects, the control is a negative control sample obtained from a healthy patient. In other aspects, the control is a positive control sample obtained from a patient diagnosed with SUDV or EBOV infection. In still other aspects, the control is a historical control or standard reference value or range of values (such as a previously tested control sample, such as a group of SUDV or EBOV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a protein (for example, an antibody or portion thereof (such as a variable region) that specifically binds SUDV and/or EBOV GP) that comprises a sequence that is degenerate as a result of the genetic code. There are twenty natural amino acids, most of which are specified by more than one codon. Therefore, all degenerate nucleotide sequences are included as long as the amino acid sequence of the antibody encoded by the nucleotide sequence is unchanged.

Detectable label: A detectable molecule (also known as a detectable marker) that is conjugated directly or indirectly to a second molecule, such as an antibody, to facilitate detection of the second molecule. For example, the detectable label can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable labels include fluorophores, chemiluminescent agents, enzymatic linkages, radioactive isotopes, nucleic acids (such as DNA barcodes), and heavy metals or compounds (for example super paramagnetic iron oxide nanocrystals for detection by MRI). In one example, a “labeled antibody” refers to incorporation of another molecule in the antibody. For example, the label is a detectable label, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides (such as antibodies) are known and may be used. Examples of labels for polypeptides include, but are not limited to, the following: radioisotopes or radionuclides (such as ³S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance. Methods for using detectable labels and guidance in the choice of detectable labels appropriate for various purposes are discussed for example in Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Ebolavirus: A genus of enveloped, non-segmented, negative-sense, single-stranded RNA viruses that causes Ebolavirus disease (EVD), formerly known as Ebola hemorrhagic fever (EHF), in humans. Ebolaviruses spread through human-to-human transmission, with infection resulting from direct contact with blood, secretions, organs or other bodily fluids of infected people, and indirect contact with environments contaminated by such fluids.

The symptoms of Ebolavirus infection and EVD are well-known. Briefly, in humans, Ebolaviruses have an initial incubation period of 2 to 21 days (7 days on average, depending on the Ebolavirus species) followed by rapid onset of non-specific symptoms such as fever, extreme fatigue, gastrointestinal complaints, abdominal pain, anorexia, headache, myalgias and/or arthralgias. These initial symptoms last for about 2 to 7 days after which more severe symptoms related to hemorrhagic fever occur, including hemorrhagic rash, epistaxis, mucosal bleeding, hematuria, hemoptysis, hematemesis, melena, conjunctival hemorrhage, tachypnea, confusion, somnolence, and hearing loss. In general, the symptoms last for about 7 to 14 days after which recovery may occur. Death can occur 6 to 16 days after the onset of symptoms. People are infectious as long as their blood and secretions contain the virus, which in some instances can be more than 60 days.

Immunoglobulin M (IgM) antibodies to the virus appear 2 to 9 days after infection whereas immunoglobulin G (IgG) antibodies appear approximately 17 to 25 days after infection, which coincides with the recovery phase. In survivors of EVD, both humoral and cellular immunity are detected, however, their relative contribution to protection is unknown.

Six distinct species of Ebolavirus are known, including Zaire ebolavirus (EBOV), Sudan ebolavirus (SUDV), Bundibugyo ebolavirus (BDBV), Reston ebolavirus (RESTV), Tar Forest ebolavirus (TAFV), and Bombali ebolavirus (BOMV). Bundibugyo ebolavirus, Sudan ebolavirus, and Zaire ebolavirus have been associated with large outbreaks of EVD in Africa and reported case fatality rates of up to 90%.

The genome of Ebolaviruses includes about 19 kb, which encode seven structural proteins including NP (a nucleoprotein), VP35 (a polymerase cofactor), VP30 (a transcriptional activator), VP24, L (a RNA polymerase), and GP (a glycoprotein).

Effective amount (or therapeutically effective amount): A quantity of a specific substance sufficient to achieve a desired effect in a subject to whom the substance is administered. For instance, this can be the amount necessary to inhibit, prevent or treat a SUDV or EBOV infection, or to measurably alter outward symptoms of the infection.

In some aspects, a therapeutically effective amount of a disclosed antibody (or bispecific antibody) that binds to SUDV or EBOV GP is an amount necessary to reduce or inhibit a SUDV or EBOV infection (for example, as measured by infection of cells, or by number or percentage of subjects infected by SUDV or EBOV, or by an increase in the survival time of infected subjects, or by reduction in symptoms associated with the infection) by a desired amount, for example by at least 10%, at least 20%, at least 50%, at least 60%, at least 70%, at least 80%, at least 90%, at least 95%, at least 98%, or even at least 100% (elimination or prevention of detectable SUDV or EBOV infection), as compared to a suitable control.

The effective amount (or therapeutically effective amount) of an antibody disclosed herein that is administered to a subject to inhibit SUDV and/or EBOV infection will vary depending upon a number of factors associated with that subject, for example the overall health and/or weight of the subject. An effective amount can be determined by varying the dosage and measuring the resulting response, such as, for example, a reduction in SUDV or EBOV titer. Effective amounts also can be determined through various in vitro, in vivo or in situ immunoassays.

An effective or therapeutically effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the bispecific antibody can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted. Effector molecules can include, for example, polypeptides, small molecules, drugs, toxins, therapeutic agents, detectable labels, nucleic acids, lipids, nanoparticles, carbohydrates or recombinant viruses. In one non-limiting example, the effector molecule is a toxin. Some effector molecules may have or produce more than one desired effect.

Epitope: An antigenic determinant. Epitopes are particular chemical groups or peptide sequences on a molecule that are antigenic (elicit a specific immune response). An antibody specifically binds a particular antigenic epitope on a polypeptide (such as GP). In some examples a disclosed antibody specifically binds to an epitope on SUDV or EBOV GP. In some examples, a disclosed bispecific antibody specifically binds to two different epitopes on SUDV or EBOV GP (a first antigen binding portion of the bispecific antibody binds a first epitope of GP and a second antigen binding portion of the bispecific antibody binds a second epitope of GP).

Expression control sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements that are sufficient to render promoter-dependent gene expression controllable for cell-type specific, tissue-specific, or inducible by external signals or agents; such elements may be located in the 5′ or 3′ regions of the gene. Both constitutive and inducible promoters are included. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Glycoprotein (GP): The virion-associated transmembrane glycoprotein of Ebolavirus (such as GP of SUDV or EBOV) is initially synthesized as a precursor protein of about 676 amino acids in size, designated GP₀. Individual GP₀ polypeptides form a homotrimer and undergo glycosylation and processing to remove the signal peptide, as well as cleavage by a cellular protease between approximately positions 501/502 (from the initiating methionine) to generate separate GP₁ and GP₂ polypeptide chains, which remain associated via disulfide bonds as GP₁/GP₂ protomers within the homotrimer. The extracellular GP₁ trimer (approximately 153 kDa) is derived from the amino-terminal portion of the GP₀ precursors. The GP₂ trimer (approximately 59 kDa), which includes extracellular, transmembrane, and cytosolic domains, is derived from the carboxyl-terminal portion of the GP₀ precursors. GP₁ is responsible for attachment to new host cells while GP₂ mediates fusion with those cells. GP, contains a mucin-like domain from position 309-501 that is dispensable for infection. Given this, the domain is often removed in order to more efficiently produce viruses and proteins for assays and is referred to as GP_{dMuc}, or GPΔMuc.

Comparisons of the predicted amino acid sequences for the GPs of the different species of Ebolavirus show conservation of amino acids in the amino-terminal and carboxy-terminal regions with a highly variable region in the middle of the protein (Sanchez et al., Virus Res. 29(3): 215-240, 1993; Sanchez et al. Proc. Natl. Acad. Sci. U.S.A., 93(8): 3602-3607, 1996). The GPs of the Ebolaviruses are highly glycosylated and contain both N-linked and O-linked carbohydrates that contribute up to 50% of the molecular weight of the protein. Most of the glycosylation sites are found in the central variable region of GP.

Heterologous: Originating from a separate genetic source or species. For example, a promoter can be heterologous to an operably linked nucleic acid sequence.

IgG: A polypeptide belonging to the class or isotype of antibodies that are substantially encoded by a recognized immunoglobulin gamma gene. In humans, this class comprises IgG₁, IgG₂, IgG₃, and IgG₄. In mice, this class comprises IgG₁, IgG₂a, IgG_{2b}, IgG₃.

Immune complex: The binding of an antibody to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, X-ray and affinity chromatography. Immunological binding properties of selected antibodies may be quantified using well-known methods.

Isolated: A biological component (such as a nucleic acid, peptide, protein or protein complex, for example an antibody) that has been substantially separated, produced apart from, or purified away from other biological components in the cell of the organism in which the component occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Linker: A bi-functional molecule that can be used to link two molecules into one contiguous molecule, for example, to link an effector molecule to an antibody. Non-limiting examples of peptide linkers include glycine-serine linkers. The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an antibody or antibody fragment. The linkage can be either by chemical or recombinant means. “Chemical means” refers to a reaction between the antibody moiety and the effector molecule such that there is a covalent bond formed between the two molecules to form one molecule.

Neutralizing antibody: An antibody (or bispecific antibody) that reduces the infectious titer of an infectious agent by binding to a specific antigen on the infectious agent, such as a virus (e.g., SUDV or EBOV). In some aspects, an antibody or bispecific antibody that is specific for SUDV GP neutralizes the infectious titer of SUDV. For example, an antibody or bispecific antibody that neutralizes SUDV may interfere with the virus by binding it directly and limiting entry into cells. Alternately, a neutralizing antibody may interfere with one or more post-attachment interactions of the pathogen with a receptor, for example, by interfering with viral entry using the receptor. In some aspects, an antibody or bispecific antibody that specifically binds to SUDV GP and neutralizes SUDV inhibits infection of cells, for example, by at least 50%, by at least 60%, by at least 70%, by at least 80% or by at least 90%, compared to a control antibody. In some aspects, an antibody, such as a bispecific antibody, that specifically binds to an SUDV GP can neutralize two or more (such as three, four, five, or more) species of Ebolavirus.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed antibodies.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some aspects, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Recombinant: A recombinant nucleic acid is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several aspects, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The identity between two or more nucleic acid sequences, or two or more amino acid sequences, is expressed in terms of the identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75%, for example at least about 80%, 85%, 90%, 91%, 92%, 93%, 94%, 95%, 96%, 97%, 98% or 99% sequence identity counted over the full-length alignment with the amino acid sequence of interest.

Methods of alignment of sequences for comparison are well known in the art. Various programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics. 5(2):151-3, 1989; Corpet, Nucleic Acids Res. 16(22):10881-10890, 1988; Huang et al. Bioinformatics. 8(2):155-165, 1992; and Pearson, Methods Mol. Biol. 24:307-331, 1994. Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp. blastn, blastx, tblastn, and tblastx. Blasin is used to compare nucleic acid sequences, while blastp is used to compare amino acid sequences. Additional information can be found at the NCBI web site.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or bispecific antibody, refers to a binding reaction that determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example SUDV GP) and does not bind in a significant amount to other proteins present in the sample or subject. Specific binding can be determined by methods known in the art. See Greenfield (Ed.), Antibodies: A Laboratory Manual, 2^{nd} ed. New York: Cold Spring Harbor Laboratory Press, 2014, for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide-ligand interaction or an antibody-antigen interaction. For example, for the bimolecular interaction of an antibody or antigen binding fragment and an antigen it is the concentration of the individual components of the bimolecular interaction divided by the concentration of the complex.

An antibody (or antigen-binding fragment) that specifically binds to an epitope on SUDV or EBOV GP is an antibody that binds substantially to SUDV or EBOV GP, including cells or tissue expressing SUDV or EBOV GP, substrates to which the SUDV or EBOV GP is attached, or SUDV or EBOV GP in a biological specimen. It is recognized that a certain degree of non-specific interaction may occur between an antibody and a non-target (such as a cell that does not express SUDV or GP). Typically, specific binding results in a much stronger association between the antibody and protein or cells bearing the antigen than between the antibody and protein or cells lacking the antigen. Specific binding typically results in greater than 2-fold, such as greater than 5-fold, greater than 10-fold, or greater than 100-fold increase in the amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats (e.g., ELISA, BLI, SPR and flow cytometry) are appropriate for selecting antibodies or other ligands specifically immunoreactive with a particular protein.

Subject: Living multicellular vertebrate organisms, a category that includes human and non-human mammals. In some examples, the subject is a human. In other examples, the subject is a non-human primate (NHP). In some examples, the subject is a subject with an SUDV or EBOV infection or at risk of an SUDV or EBOV infection.

Synthetic: Produced by artificial means in a laboratory, for example a synthetic nucleic acid or protein (for example, an antibody) can be chemically synthesized in a laboratory.

Treating or inhibiting a disease or condition: Reducing the full development of a disease or condition in a subject, for example, reducing the full development of EVD in a subject who has a SUDV or EBOV infection (e.g., reducing viremia), and/or reducing SUDV/EBOV infection in a subject or in a population of subjects at risk thereof. This includes neutralizing, antagonizing, prohibiting, preventing, restraining, slowing, disrupting, stopping, or reversing progression, severity or spread of the disease or condition.

Treating a disease or condition (such as a SUDV or EBOV infection) refers to a therapeutic intervention that ameliorates a sign or symptom of a disease or pathological condition after it has begun to develop. The term “ameliorating” refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. Inhibiting a disease or condition refers to a prophylactic intervention administered before the disease or condition has begun to develop (for example a treatment initiated in a subject at risk of an EBOV or SUDV infection, but not infected by an EBOV or SUDV) that reduces subsequent development of the disease or condition, and also ameliorates one or more signs or symptoms of the disease or condition following development. A “prophylactic” treatment is a treatment administered to a subject who does not exhibit signs of a disease for the purpose of reducing the risk of developing pathology.

In some aspects, an antibody that specifically binds to SUDV or EBOV GP inhibits infection of a human subject by a SUDV or EBOV, for example, by at least 25%, at least 50%, at least 60%, at least 70%, at least 80%, at least 90%, or at least 95%, compared to a control or compared to the absence of treatment.

Under conditions sufficient for: A phrase that is used to describe any environment that permits a desired activity. In some examples, the desired activity is formation of an immune complex. In other examples, the desired activity is treatment of a SUDV or EBOV infection.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. In some aspects, a viral vector comprises a nucleic acid molecule encoding a disclosed antibody or bispecific that specifically binds to SUDV and/or EBOV GP. Viral vectors include, but are not limited to, lentiviral vectors, adenovirus vectors and adeno-associated virus (AAV) vectors.

### IV. Monoclonal Antibodies Specific for Sudan and Zaire Ebolavirus Glycoprotein

Monoclonal antibodies that specifically bind SUDV and/or EBOV GP with nanomolar affinity are described. The disclosed monoclonal antibodies were isolated through nested PCR of the heavy and light chain immunoglobulin genes from GP-positive, single cell-sorted B cells from PBMC samples of human and NHP subjects previously immunized with EBOV and/or SUDV glycoprotein. Several of the monoclonal antibodies (and combinations thereof) are shown herein to potently neutralize infection by SUDV GP- and/or EBOV GP-pseudotyped lentiviruses (FIGS. 2A, 2B, 11, 17 and 21) and/or provide potent protection against SUDV challenge in a NHP model (FIGS. 8, 9 and 15). Thus, the disclosed monoclonal antibodies, bispecific monoclonal antibodies and compositions thereof can be used for treating, inhibiting and detecting infection by Ebolaviruses, such as SUDV and EBOV.

### A. Antibody Sequences

The nucleotide and amino acid sequences of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L and 396L antibodies are provided below. The location of each CDR, as determined by IMGT, is underlined in each amino acid sequence. The CDR residues are also listed in Table 18 below. Although the CDR locations were identified using IMGT, a skilled person understands that other numbering schemes, such as Chothia or Kabat, can also be used to determine the boundaries of each CDR.

316L VH domain
(SEQ ID NO: 1)
gaagagagattggtggagtctgggggaggcctggtccagcctggggggtccctgagactctcgtgtgcagcctctggattcacttttggcgattatg
ccatgcactgggtccgccaagttcctgggaagagtctcgagtggctctctggtataagttggagtggttatacgacatactctgcagactccgtcaa
gggccgattcaccatctccagagacaacgccaagaattccctgtatctgcaaatgaaccgcctgagacctgaggacacaggcctctattactgtgtg
accttgataggggcgattggaattaaagactcattggatgtctggggccggggagttctggtcaccgtctcctca
316L VH domain
(SEQ ID NO: 2)
EERLVESGGGLVQPGGSLRLSCAASGFTFGDYAMHWVRQVPGKSLEWLSGISWSGYTTYSADSVK
GRFTISRDNAKNSLYLQMNRLRPEDTGLYYCVTLIGAIGIKDSLDVWGRGVLVTVSS
316L VL domain
(SEQ ID NO: 3)
cagtctgtgctgacgcagccgccctcagtgtctggggcgcccgggcagagggtcaccgtctcctgctctgggagcagctccaacattggggcgggta
attatgtacagtggtaccagcaacttccaggaacggcccccaaagtcctcatctatcaaactgagaagcgaccctcaggcacttctgatcgattctc
tggctccaagtctgatacctcggcctccctgaccatcaatggactccagtctgaggatgaggctgattattactgccaggtctatgacagcaatttg
aatggttgggtattcggaggagggacccggctgacagtacta
316L VL domain
(SEQ ID NO: 4)
QSVLTQPPSVSGAPGQRVTVSCSGSSSNIGAGNYVQWYQQLPGTAPKVLIYQTEKRPSGTSDRESGS
KSDTSASLTINGLQSEDEADYYCQVYDSNLNGWVFGGGTRLTVL
380L VH domain
(SEQ ID NO: 5)
gaagtgcagttggtggagtctgggggaggcctggtccagcctggggggtccctgacactctcgtgtgcagcctctggattcacctttggtgattatg
tcatgcactgggtccgccaagttccagggaggggtctagagtggctctctgctgtaagttggagtggctacaccacatactctgcagactccgtcaa
gggccgattcaccatctccagagacaacgccaagaactccctctatctacaaatgagccgcctgggaactgcggacacaggcctctattactgtgta
accctgataggggcgattggaatacgcgactccttcgatgcctggggccggggagttctggtcaccgtctcctca
380L VH domain
(SEQ ID NO: 6)
EVQLVESGGGLVQPGGSLTLSCAASGFTFGDYVMHWVRQVPGRGLEWLSAVSWSGYTTYSADSV
KGRFTISRDNAKNSLYLQMSRLGTADTGLYYCVTLIGAIGIRDSFDAWGRGVLVTVSS
380L VL domain
(SEQ ID NO: 7)
cagtctgtgctgacgcagccgccctcaatgtctggggcgcccgggcagagggtcaccgtctcctgcacggggagcagctccaacattggagcgggta
attatgtgcagtggtaccagcaacttccaggaacggcccccaaagtcctcatctatcaaactgataagcgaccctcaggtatttctgatcgattctc
tggctccaagtctggtacctcagcctccctgaccatcagtggactccagtctgaggatgaggctgattattactgccaggtctatgacagcagttt
gaatggttgggtattcggaggagggacccggctgacagtacta
380L VL domain
(SEQ ID NO: 8)
QSVLTQPPSMSGAPGQRVTVSCTGSSSNIGAGNYVQWYQQLPGTAPKVLIYQTDKRPSGISDRESGS
KSGTSASLTISGLQSEDEADYYCQVYDSSLNGWVFGGGTRLTVL
291S VH domain
(SEQ ID NO: 9)
caggtgcagctgcaggagtcggggccaggactggtgaagccttcacagaccctgtccctcacctgcactgtctctggtggctccttcagtagcggtt
tctactggagttggatccgccagcgcccagggaagggcctggagtgggtcgggtacacctattacagtgggagcacctactacaacccgtccctcca
gagtcgagtcaccatatcagtggacacgtctaagaaccagttctccctgaacctgagctctgtgactgccgcggacacggccgtgtatcactgtgcg
agagccgactacggtggcctctttgactactggggccagggaattctggtcaccgtctcctca
291S VH domain
(SEQ ID NO: 10)
QVQLQESGPGLVKPSQTLSLTCTVSGGSFSSGFYWSWIRQRPGKGLEWVGYTYYSGSTYYNPSLQS
RVTISVDTSKNQFSLNLSSVTAADTAVYHCARADYGGLFDYWGQGILVTVSS
291S VL domain
(SEQ ID NO: 11)
cagtctgtgctgactcagccgtcttccctctctgcatctcctggagcatcagccagtctcacctgcaccttgcgcagtggcatcaatgttggtactt
acaggatattctggtaccagcagaagccagggagtcctccccagtttctcctgaggtacaactcagactcagataagcagcagggctctggagtccc
cagccgcttctctggatccaaagatgcttcggccaatgcagggattttactcatctctgggctccagtctgaggatgaggctgactattactgtatg
atttggcacaccagcgcttgggtgttcggcggagggaccaagctgaccgtccta
291S VL domain
(SEQ ID NO: 12)
QSVLTQPSSLSASPGASASLTCTLRSGINVGTYRIFWYQQKPGSPPQFLLRYNSDSDKQQGSGVPSRF
SGSKDASANAGILLISGLQSEDEADYYCMIWHTSAWVFGGGTKLTVL
545S VH domain
(SEQ ID NO: 13)
caggtgcagctgcaggagtcggggccaggactggtgaagccttcacagaccctgtccctcacctgcactgtctctggtggctccatcagccgtgctg
gttcctactggagctggatccgccagcacccagggaagggcctggagtggattggctacatctcttacagtgggagcacctactacaacccgtccct
cgagagtcgagttaccatgtcactagacacgtctaagagccagttctcccttaagttgagctctgtgactgccgcggacacggccgtctattattgt
gcgagagaggaccgctcgggcttctacggtttggacgtctggggccaagggaccacggtcaccgtctcctca
545S VH domain
(SEQ ID NO: 14)
QVQLQESGPGLVKPSQTLSLTCTVSGGSISRAGSYWSWIRQHPGKGLEWIGYISYSGSTYYNPSLES
RVTMSLDTSKSQFSLKLSSVTAADTAVYYCAREDRSGFYGLDVWGQGTTVTVSS
545S VL domain
(SEQ ID NO: 15)
cagtctgtgctgactcagccgtcttccctctctgcatctcctggagcaacagccagtctcacttgcaccttgcgcagtggcatcaatgttgatacct
acaggatatactggtaccagcagaagccagggagtcctccccagtatctcctgaggtacaaatcagactcggataaacatcagggctctggagtccc
cagccgcttctctggatccaaagatgcttcggccaatgcagggattttactcatctctgggctccagtctgcggatgaggctgactattactgtatg
atatggcacagcggcgcttgggtgttcggcggagggaccaagctgaccgtccaa
545S VL domain
(SEQ ID NO: 16)
QSVLTQPSSLSASPGATASLTCTLRSGINVDTYRIYWYQQKPGSPPQYLLRYKSDSDKHQGSGVPSR
FSGSKDASANAGILLISGLQSADEADYYCMIWHSGAWVFGGGTKLTVQ
523S VH domain
(SEQ ID NO: 17)
caggtgcagctggtgcagtctggggctgagttgaagaagcctggggcctcagtgaaggtctcctgcaaggcttctggatacaccttcagcgaccact
atgtgcactgggtgcgacaggcccctggacaagggcttgagtggatgggacggatcaaccctaacagtggtggcacaaactatgcacagaggtttct
gggcagggtcaccatgaccagggacgcgtccatcagcacagcctacctggacctgagcagtctgagatctgacgacacggccgtctattattgtgcg
agaagtagcagcagttggtcggggcactactactactacatggacgtctggggcaaagggaccacggtcaccgtctcgtca
523S VH domain
(SEQ ID NO: 18)
QVQLVQSGAELKKPGASVKVSCKASGYTFSDHYVHWVRQAPGQGLEWMGRINPNSGGTNYAQRF
LGRVTMTRDASISTAYLDLSSLRSDDTAVYYCARSSSSWSGHYYYYMDVWGKGTTVTVSS
523S VL domain
(SEQ ID NO: 19)
cagtctgtgctgacgcagccgccctcagtgtctgcggccccaggacagagggtcaccatctcctgctctggaagcacctccaacattgcgagtaatt
ttgtatcctggtaccagcagctcccaggaacagcccccaaactcctcatttatgataatcataagcgaccctcagggattcctgaccgattctctgg
ctccaagtctggcacgtcagccaccctgggcatcaccggactccagactggggacgaggccgattattactgcggaacatgggatagcagcctgagt
gctggggtcttcggcggagggaccaaggtgaccgtccta
523S VL domain
(SEQ ID NO: 20)
QSVLTQPPSVSAAPGQRVTISCSGSTSNIASNFVSWYQQLPGTAPKLLIYDNHKRPSGIPDRFSGSKS
GTSATLGITGLQTGDEADYYCGTWDSSLSAGVFGGGTKVTVL
573S VH domain
(SEQ ID NO: 21)
gaagtgcagttggtggagtctgggggaggcctggtcaagccgggggggtccctgagactctcctgtgcagcctctggattcaccttcagtagttata
ccatgaactgggtccgccaggctccagggaaggggctggagtgggtctcatgcattaggagtagcagtagtgacaaatactacgcagactcagtgaa
gggccgattcaccatctccagagacaacaccaagaactcactgtatctgcaaatgaacagcctgagagccgacgacacggctgtgtattactgtgcg
agagatctaggcggtgctaataaccgCtactacttcgactatggtatggacgtctggggccaagggaccacggtcaccgtctcctca
573S VH domain
(SEQ ID NO: 22)
EVQLVESGGGLVKPGGSLRLSCAASGFTFSSYTMNWVRQAPGKGLEWVSCIRSSSSDKYYADSVK
GRFTISRDNTKNSLYLQMNSLRADDTAVYYCARDLGGANNRYYFDYGMDVWGQGTTVTVSS
573S VL domain
(SEQ ID NO: 23)
gaaatagtgatgacgcagtctccagccaccctgtctgtgtctccagggggaagagccaccctctcctgcagggccagtcagagtgtttacaccaact
tagcctggtaccagcagaaacctggccaggctcccaggctcctcatctatgatgcatccaccagggccactggtatcccagccaggttcagtggcag
tgggtctgggacagagttcactctcaccatcagcagcctgcagtgtgaggattttgcagtttattactgtcagcactataataactggcccctgtac
acttttggccaggggaccaagctggagatcaaa
573S VL domain
(SEQ ID NO: 24)
EIVMTQSPATLSVSPGGRATLSCRASQSVYTNLAWYQQKPGQAPRLLIYDASTRATGIPARFSGSGS
GTEFTLTISSLQCEDFAVYYCQHYNNWPLYTFGQGTKLEIK
541S VH domain
(SEQ ID NO: 25)
caggtgcagctggtgcagtctggagctgaagtgaagaagcctggggcctcagtgaaggtctcctgcaaggcttctggttacacctttaccaactatg
ccatcacctgggtgcgacaggcccctggacaagggcttgagtggatgggatggatcagcgctttcaatggtcaaataaactatgcacagaagttcca
gggcagagtcgccatgaccacagacacatccacgagcacagcctacatggagctgaggagcctgagatctgacgacacggccgtctattactgtgcg
agagccccccctatgtggttctctggggccaaatttgactactggggccagggaaccctggtcaccgtctcctca
541S VH domain
(SEQ ID NO: 26)
QVQLVQSGAEVKKPGASVKVSCKASGYTFTNYAITWVRQAPGQGLEWMGWISAFNGQINYAQKF
QGRVAMTTDTSTSTAYMELRSLRSDDTAVYYCARAPPMWESGAKFDYWGQGTLVTVSS
541S VL domain
(SEQ ID NO: 27)
cagtctgtgctgactcagccaccctcagtgtctgggacccccgggcagagggtcaccatctcttgttctggaagcagctccaacatCggaagcaata
ctgtaaactggtaccagcagctcccaggaacggcccccaaactCctcatctatagtaatgatcagcggccctcaggggtccctgaccgattctctgg
ctccaagtctggcacctctgcctccctggccatcagtgggctccggtccgaggatgaggctgattattactgtgcagcatgggatgaaaccctgagg
agggtgttcggcggagggaccaggctgaccgtccta
541S VL domain
(SEQ ID NO: 28)
QSVLTQPPSVSGTPGQRVTISCSGSSSNIGSNTVNWYQQLPGTAPKLLIYSNDQRPSGVPDRFSGSKS
GTSASLAISGLRSEDEADYYCAAWDETLRRVFGGGTRLTVL
294S VH domain
(SEQ ID NO: 29)
caggtgcagctgcaggagtcgggGccaggactggtgaagccttcacagaccctgtccctcacctgcactgtctctggtggctccatcagcagtggtg
gttaccactggagctggatccgccagcacccagggaagggcctggagtggattgggaacatctattacaatgggcgcacctactataatccgtccct
caagagtcgagttaccatatcagtagacacgtctgagaacgaattctccctgaagttgagctctgtgactgccgcggacacggccgtgtattactgc
gcgagagatcggaggaggcctagaatcgggcacgactacggtatggacgtctggggccaagggaccaaggtcatcgtctcctca
294S VH domain
(SEQ ID NO: 30)
QVQLQESGPGLVKPSQTLSLTCTVSGGSISSGGYHWSWIRQHPGKGLEWIGNIYYNGRTYYNPSLK
SRVTISVDTSENEFSLKLSSVTAADTAVYYCARDRRRPRIGHDYGMDVWGQGTKVIVSS
294S VL domain
(SEQ ID NO: 31)
gacatccagatgacccagtctccatcctccctgtctgcatctgtaggagacagagtcaccatctcttgccgggcaagtcagagcattagcaactttt
taaattggtatcagcagaaaccagggcaagcccctaaactcctgatctacactgcatccaatttgcaaagtggggtcccatcaaggttcagtggcag
tggatctgggacagatttcactctcaccatcagcagtctgcaacctgaagattttgcaacttactactgtcaacagatttacaataccggcatgtac
tcttttggccaggggaccaagctggagatcaaa
294S VL domain
(SEQ ID NO: 32)
DIQMTQSPSSLSASVGDRVTISCRASQSISNFLNWYQQKPGQAPKLLIYTASNLQSGVPSRFSGSGSG
TDFTLTISSLQPEDFATYYCQQIYNTGMYSFGQGTKLEIK
241S VH domain
(SEQ ID NO: 33)
caggtgcagctggtacaatctgggtctgagttgaagaagcctggggcctcagtgaaggtttcctgcaaggcttctggatacaccttcacaagctata
ctgtgaattgggtgcgacaggcccctggacaagggcttgagtggatgggatggatcaacaccaacactgggaacccaacgtatgcccagggcttcac
aggacggtttgtcttctcctcggacacctctgtcagcacggcatatctgcagatcagcgacctaaaggctgaggacacagccatgtattactgtgcg
agacacgtcgcatacgatctttggagtggtcacacggttacctttgaccactggggccagggaaccctggtcacagtctcctca
241S VH domain
(SEQ ID NO: 34)
QVQLVQSGSELKKPGASVKVSCKASGYTFTSYTVNWVRQAPGQGLEWMGWINTNTGNPTYAQGF
TGRFVFSSDTSVSTAYLQISDLKAEDTAMYYCARHVAYDLWSGHTVTFDHWGQGTLVTVSS
241S VL domain
(SEQ ID NO: 35)
cagtctgccctgactcagcctccctccgcgtccgggtctcctggacagtcagtcaccatctcctgcactggaaccagcagtgacgttggtgcttata
actacgtctcctggtaccagcagcacccaggcaaagcccccaaactcatcatttatgaggtcactaagCggccctcaggggtccctgatcgcttctc
tggctccaagtctggcaacacggcctccctgaccgtctctgggctccaggctgaggatgaggctgattattactgcagctcaaaagcaggcggaaaa
acttatgtcttcggaactgggaccaaggtcaccgtccta
241S VL domain
(SEQ ID NO: 36
QSALTQPPSASGSPGQSVTISCTGTSSDVGAYNYVSWYQQHPGKAPKLIIYEVTKRPSGVPDRFSGS
KSGNTASLTVSGLQAEDEADYYCSSKAGGKTYVFGTGTKVTVL
354S VH domain
(SEQ ID NO: 37)
caggtgcagctggtgcagtctggggctgaggtgaagaagcctggggcctcagtgaaggtttcctgcaaggcatctggatacagcttcgccagttact
atatacactgggtgcgacaggcccctggacaagggcttgagtggatgggaataatcaaccctagtggtggtaccacaagctacgcacagaagttcca
gggcagagtcaccatgaccagggacacgtccacgagcacagtctacatggagctgagcagcctgggatctgaggacacggccgtgtattactgtgcg
agaatagtcgatcgagttacgatatatctgaatggggttgataactactggggccagggaaccctggtcaccgtctcctca
354S VH domain
(SEQ ID NO: 38)
QVQLVQSGAEVKKPGASVKVSCKASGYSFASYYIHWVRQAPGQGLEWMGIINPSGGTTSYAQKFQ
GRVTMTRDTSTSTVYMELSSLGSEDTAVYYCARIVDRVTIYLNGVDNYWGQGTLVTVSS
354S VL domain
(SEQ ID NO: 39)
gaaatagtgatgacgcagtctccagccaccctgtctgtgtctccaggggaaagagtcaccctctcctgcagggccagtcagagcgttagcagcaact
tagcctggttccagcagacacctggccaggctcccaggctcctcatctatggtgcatccaccagggcctctggtgtcccagccaggttcagtggcag
tgggtctgggacagaattcactctcaccatcagcagcctgcagtctgaagattttgcagtctattactgtctgcagtataataattggcctccgggt
tggaagtacggttttggccaggggaccaagctggagatcaaa
354S VL domain
(SEQ ID NO: 40)
EIVMTQSPATLSVSPGERVTLSCRASQSVSSNLAWFQQTPGQAPRLLIYGASTRASGVPARFSGSGS
GTEFTLTISSLQSEDFAVYYCLQYNNWPPGWKYGFGQGTKLEIK
233S VH domain
(SEQ ID NO: 41)
gaagtgcagctggtggagtctgggggaggcttgggacagcctggggggtccctgagactctcctgtgcagcctctggattcaagttcaaaatctttg
gcatgaactgggtccgccaggctccagggaaggggctggagtgggtttcatacattagtagtagaagtagtatcatatattatgcagactctgtgaa
gggccggttcaccatctccagagacgatgccacgaattcactgtttctgcaaatgaacagcctgagagacgaggacacggctatgtattactgtgtg
agagattacaatttgattgacttcgtttgggggagtctatttgactactggggccagggaatcctggtcaccgtctcctca
233S VH domain
(SEQ ID NO: 42)
EVQLVESGGGLGQPGGSLRLSCAASGFKFKIFGMNWVRQAPGKGLEWVSYISSRSSIIYYADSVKG
RFTISRDDATNSLFLQMNSLRDEDTAMYYCVRDYNLIDFVWGSLFDYWGQGILVTVSS
233S VL domain
(SEQ ID NO: 43)
gccatccggatgacccagtctccctcctcactctcagcatctacaggagacagcgtcaccatcacttgtcgggcgagtcagaatattgccagttatt
tagcctggtatcagcaaataccaggaaaagcccctaagctcctgatttatgttgcatccactttgcaaagtggggtcccatccaggttcagcggcag
tggatctgggacagatttcactctcaccatcagctccctgcagtctgaagactttgcaacttattactgtcagcagtattatcgttaccctccgacg
ttcggccaggggaccaaggtggaaatcaaa
233S VL domain
(SEQ ID NO: 44)
AIRMTQSPSSLSASTGDSVTITCRASQNIASYLAWYQQIPGKAPKLLIYVASTLQSGVPSRFSGSGSG
TDFTLTISSLQSEDFATYYCQQYYRYPPTFGQGTKVEIK
503S VH domain
(SEQ ID NO: 45)
caggtgcagctggtgcagtctggggctgaggtgaagaagcctggggcctcagtgagggtttcctgcaaggcatctggatacaccttcaccagctact
atatgcactgggtccgacaggcccctggacaagggcttgagtggatgggacagatcaaccctagtggcggcaccacaaggtacgaacggaagttcca
gggcagaatcaccgtgacccgggacacgtccacgagcacagtctacttggaactgagcagcctgagatctgaggacacggccagttatttttgtgcg
tgtcttcgggataaggtgacgtattacgatctctggagtggagtGgactccgtttggggccagggaaccctggtcaccgtctcctca
503S VH domain
(SEQ ID NO: 46)
QVQLVQSGAEVKKPGASVRVSCKASGYTFTSYYMHWVRQAPGQGLEWMGQINPSGGTTRYERKF
QGRITVTRDTSTSTVYLELSSLRSEDTASYFCACLRDKVTYYDLWSGVDSVWGQGTLVTVSS
503S VL domain
(SEQ ID NO: 47)
gacatccagatgacccagtctccatcctcactgtctgcatctgtaggagacagagtcaccatcacttgtcgggcgagtcaggatattagcacctggt
tagcctggtatcagcagaaaccagagaaagcccctaagtccctgatctatgctgcatccactttggaaactggggtcccatccaggttcagcggcag
tggatctgggacagatttcactctcaccatcagcagcctgcagcctgaagattttgcaacttattactgcgagcagtataatggtttccctcggact
tttggccaggggaccaagctggacatcaaa
503S VL domain
(SEQ ID NO: 48)
DIQMTQSPSSLSASVGDRVTITCRASQDISTWLAWYQQKPEKAPKSLIYAASTLETGVPSRFSGSGSG
TDFTLTISSLQPEDFATYYCEQYNGFPRTFGQGTKLDIK
203S VH domain
(SEQ ID NO: 49)
caggtccagctggtgcagtctggggctgaggtgaagaagcctgggtcctcggtgaaggtctcctgcaaggcttctggaggcaccttcagcagttatg
ctatcagctgggtgcgacaggcccctggacaagggcttgagtggttgggaaggatcctccctgcccttggtttagcaaactacgcacagaagtacca
ggacagagtcaagattaccgcggacaaatccacgagcacagcctacatggagctgagcagactgggatctgaggacacggccgtgtattactgtgcg
agagatcgctgggtgggagctgcccggcgggggcagtactttgactactggggccagggaacccaggtcaccgtctcctca
203S VH domain
(SEQ ID NO: 50)
QVQLVQSGAEVKKPGSSVKVSCKASGGTFSSYAISWVRQAPGQGLEWLGRILPALGLANYAQKYQ
DRVKITADKSTSTAYMELSRLGSEDTAVYYCARDRWVGAARRGQYFDYWGQGTQVTVSS
203S VL domain
(SEQ ID NO: 51)
gacatccagatgacccagtctcctgccaccctgtctgcatctgtaggagacagagtcaccatcacttgccgggccagtcagagtattggtacttggt
tggcctggtatcagcagaaaccagggaaagcccctaaactgctgatctatgatgcctccagtttggaaagtggggtcccaccaaggttcagcggcag
tgggtctgaaacagaattcactctcaccatcagcagcctgcagcctgatgattttgcaacttactactgccaccagtataagagttattcgtacact
tttggccaggggaccaagctggagatcaaa
203S VL domain
(SEQ ID NO: 52)
DIQMTQSPATLSASVGDRVTITCRASQSIGTWLAWYQQKPGKAPKLLIYDASSLESGVPPRFSGSGS
ETEFTLTISSLQPDDFATYYCHQYKSYSYTFGQGTKLEIK
315S VH domain
(SEQ ID NO: 53)
gaagtgcagctggtggagtctgggggaggcgtggtccagcctgggaggtccctgagactctcctgtgcagcctctggattcaccttcaaaacctata
ctatgcactgggtccgccaggctccaggcaagggactagagtgggtggcagttataacagatgatggagttaataaaaagtacgcagactccgtgaa
gggccgattcaccatctccagagacaattccaagaacacgctgtatctgcaaatgaacagcctgagacttgaggacacggctgtgtattactgtgcg
agaagtgttgtggttctggctgcttaccctcttgactactggggccagggaaccatggtcaccgtctcctca
315S VH domain
(SEQ ID NO: 54)
EVQLVESGGGVVQPGRSLRLSCAASGFTFKTYTMHWVRQAPGKGLEWVAVITDDGVNKKYADSV
KGRFTISRDNSKNTLYLQMNSLRLEDTAVYYCARSVVVLAAYPLDYWGQGTMVTVSS
315S VL domain
(SEQ ID NO: 55)
gacatccagatgacccagtctccatcctccctgtctgcatctgtaggagacagagtcaccatcacttgccgggcaagtcagaccattagcacctatt
tacattggtatcagcagagaccagggaaagcccctaagttgttgatctatgctgcatccagtttgcaaagtggggtcccatcaagggtcagtggcag
tggatctgggacagatttcactctcaccatcaccagtctgcaacctgaagattttgcaacttactactgtctacagacttacagtcttcctctcact
ttcggcggagggaccaacgtggagatcaaa
315S VL domain
(SEQ ID NO: 56)
DIQMTQSPSSLSASVGDRVTITCRASQTISTYLHWYQQRPGKAPKLLIYAASSLQSGVPSRVSGSGSG
TDFTLTITSLQPEDFATYYCLQTYSLPLTFGGGTNVEIK
586S VH domain
(SEQ ID NO: 57)
caggtccagctggtgcagtctggggctgaggtgaagaagccggggtcctcggtgaaagtctcctgcaaggcttctggaggcaccttcagcaactatg
ctatcaactgggtgcgacaggcccctggacaagggcttgagtggatgggaaggatcatccctttccttggtttagcaaactacgcacagaagctcca
gggcagagtcacgattaccgcggacaaatccacgagcacagcctacatggagctgagcagcctgagatctgaggacacggccctttattactgtgcg
acaccccctggatccaggtatagtggctttgactcctggggccagggaaccctggtcaccgtctcctca
586S VH domain
(SEQ ID NO: 58)
QVQLVQSGAEVKKPGSSVKVSCKASGGTFSNYAINWVRQAPGQGLEWMGRIIPFLGLANYAQKLQ
GRVTITADKSTSTAYMELSSLRSEDTALYYCATPPGSRYSGFDSWGQGTLVTVSS
586S VL domain
(SEQ ID NO: 59)
gaaattgtgttgacgcagtctccaggcaccctgtctttgtctccaggggaaagagccaccctctcctgcagggccagtcagagttttaacaactact
tagcctggtaccagcagaaacctggccaggctcccaggctcctcatcaatggtgcatccagcagggccactggcatcccagacaggttcagtggcag
tgggtctgggacagacttcactctcaccatcagcagactggagcctgaagattttgcagtgtattactgtcagcagtatggtagctcacctactttc
ggccctgggaccagagtggaaatcaaa
586S VL domain
(SEQ ID NO: 60)
EIVLTQSPGTLSLSPGERATLSCRASQSFNNYLAWYQQKPGQAPRLLINGASSRATGIPDRFSGSGSG
TDFTLTISRLEPEDFAVYYCQQYGSSPTFGPGTRVEIK
377S VH domain
(SEQ ID NO: 61)
caggtgcagctggtgcagtctggggctgaggtgaagaagcctgggtcctcggtgaaggtctcctgcaaggcctctggaggcaccttcagcaactatg
ctatcaactgggtgcgacaggcccctggacaagggcttgagtggatgggaaggaccatccccttccttggtttaacaaactacgcagagaacttcca
ggccagagtcacgattaccgcggacaagtccacgggcacgtcatacatgcagctgagcagcctcagatctgaggacacggccgtgtatttttgtgcg
aaagctgccaccagtggctggtcccccttigacaactggggccagggaaccctggtcaccgtctcctca
377S VH domain
(SEQ ID NO: 62)
QVQLVQSGAEVKKPGSSVKVSCKASGGTFSNYAINWVRQAPGQGLEWMGRTIPFLGLTNYAENFQ
ARVTITADKSTGTSYMQLSSLRSEDTAVYFCAKAATSGWSPFDNWGQGTLVTVSS
377S VL domain
(SEQ ID NO: 63)
gaaattgtgttgacgcagtctccaggcaccctgtctttgtctccaggggaaagagccaccctctcctgcagggccagtcagagtattagcagcagct
actttggctggtaccagcagaaacctggccaggctcccaggctcctcatctatggtgcatccaggagggccgctggcatcccagacaggttcagttg
cagtgggtctgggacagacttcactctcaccatcagcagactggagcctgaagattttggagtgtattactgtcagcagtatggtagctcaccgacc
ttcggccaagggaccaaggtggaaatcaaa
377S VL domain
(SEQ ID NO: 64)
EIVLTQSPGTLSLSPGERATLSCRASQSISSSYFGWYQQKPGQAPRLLIYGASRRAAGIPDRFSCSGSG
TDFTLTISRLEPEDFGVYYCQQYGSSPTFGQGTKVEIK
528S VH domain
(SEQ ID NO: 65)
gaggtgcagctggcggagtctgggggaggcgtggtccagcctgggaggtccctgagactctcctgtgcagcgtctggattcaccttcaatagctatg
gcatgcactgggtccgccaggctccaggcaaggggctggagtgggtggcagtcatctgggatgatggaagtaaaaaatattatgcagactccgtgaa
gggccgattcaccatctccagagacaattccaagaacacgctgtttctgcaaatgaacagcctgagagccgaggacacggctgtgtattactgtgcg
agagagacaattatgtttacatttggggaagttatcgttagcagtagggacaactactacatggacgtctggggcaaagggaccacggtcaccgtct
cctca
528S VH domain
(SEQ ID NO: 66)
EVQLAESGGGVVQPGRSLRLSCAASGFTFNSYGMHWVRQAPGKGLEWVAVIWDDGSKKYYADS
VKGRFTISRDNSKNTLFLQMNSLRAEDTAVYYCARETIMFTFGEVIVSSRDNYYMDVWGKGTTVT
VSS
528S VL domain
(SEQ ID NO: 67)
gaaattgtgttgacacagtctccagccaccctgtctctgtctccaggggaaagagccaccctctcctgcagggccagtcagagtgttagcaactact
tagcctggtaccaacagaaacctggccaggctcccaggctcctcatctatgatgcatctaccagggccattggcatcccagccaggttcagtggcgt
tgggtctgggacagacttcactctcaccatcagcagcctagagcctgaagattttgcagtttattactgtcagcagcgaagcaactggcctcctctc
actttcggcggagggaccaaggtggagatcaaa
528S VL domain
(SEQ ID NO: 68)
EIVLTQSPATLSLSPGERATLSCRASQSVSNYLAWYQQKPGQAPRLLIYDASTRAIGIPARFSGVGSG
TDFTLTISSLEPEDFAVYYCQQRSNWPPLTFGGGTKVEIK
246S VH domain
(SEQ ID NO: 69)
caggtccagctggtgcagtctggggctgaggtgaagaagcctgggtcctcggtgaaggtctcctgcaaggcctctggagacaccttcagcacctatg
ctatcacctgggtgcgacaggcccctggacaagggcttgagtggatgggaaggatcatccctatccttggtgtaacaaactatgcacagaagttcca
gggcagagtcacgtttaccgcggacaaatccacgatcacagcctacttggagctggtgaacctgagatctgaagacacggccgtatattactgtacg
agagatgggtacggagggcgggacctacttttaaactactactggggccagggaaccctggtcaccgtctcctca
246S VH domain
(SEQ ID NO: 70)
QVQLVQSGAEVKKPGSSVKVSCKASGDTFSTYAITWVRQAPGQGLEWMGRIIPILGVTNYAQKFQ
GRVTFTADKSTITAYLELVNLRSEDTAVYYCTRDGYGGRDLLLNYYWGQGTLVTVSS
246S VL domain
(SEQ ID NO: 71)
gaaattgtgttgacgcagtctccaggcttgctgttttggtttccaggggaaagagccaccctctcctgcagggccagccagagtgtcagcagcagca
agttagcctggtaccaacagaaacctggccaggctcccaggctcctcatctatggtgcatccagcagggcccctggcatcccagacaggttcagtgg
cagtgggtctgggacagacttcactctcaccatcaatagactggagcctgaagattttgcagtgtatttctgtcagcagtatggcagcgctcagtgg
acgttcggccaagggaccaaggtggacttcaag
246S VL domain
(SEQ ID NO: 72)
EIVLTQSPGLLFWFPGERATLSCRASQSVSSSKLAWYQQKPGQAPRLLIYGASSRAPGIPDRFSGSGS
GTDFTLTINRLEPEDFAVYFCQQYGSAQWTFGQGTKVDFK
338S VH domain
(SEQ ID NO: 73)
gaagtgcagctggtggagtctgggggaggcgtggtccagcctgggaggtccctgagactctcctgtgcagcgtctggattcaccttcagtcagtatg
gcatgcactgggtccgccaggctccaggcaaggggctggagtgcgtggcagttatatggtttgatggaagtaagaaatactatggagactccgtgaa
gggccgattcaccatctccagagacaattccaagaacacgctgtatctgcaaatgaacagcctgagagccgaggacacggctgtgtatttctgtgcg
agagagagtctgtattactatgatagtggtggtcattactacgagagagggagaattgactactggggccagggaaccctggtcaccgtctcctca
338S VH domain
(SEQ ID NO: 74)
EVQLVESGGGVVQPGRSLRLSCAASGFTFSQYGMHWVRQAPGKGLECVAVIWFDGSKKYYGDSV
KGRFTISRDNSKNTLYLQMNSLRAEDTAVYFCARESLYYYDSGGHYYERGRIDYWGQGTLVTVSS
338S VL domain
(SEQ ID NO: 75)
gacatccagatgacccagtctccatcctcactgtctgcatctgtgggagacagagtcaccatcacttgtcgggcgagtcaggacattagtaattttt
tagcctggtttcaacagaaaccagggaaagcccctaagtcccttatctatgctgcatccagtttgctaagtggggtcccatcaaagttcagcggcag
tggatctgggacagatttcactctcaccatcagcagcctgcagcctgaagattttgcaacttattactgccaacagtataatagttaccctcccact
tttggccaggggaccaagctggagatcaaa
338S VL domain
(SEQ ID NO: 76)
DIQMTQSPSSLSASVGDRVTITCRASQDISNFLAWFQQKPGKAPKSLIYAASSLLSGVPSKFSGSGSG
TDFTLTISSLQPEDFATYYCQQYNSYPPTFGQGTKLEIK
335S VH domain
(SEQ ID NO: 77)
gaggtgcagctggtggagtctggggggggcgtggtccagcctgggaggtccctgagactctcctgtgcagcgtctggattcagtttcagtaactatg
ccatgcactgggtccgccaggctccaggcaagggactggagtgggtggcagttacatggtatgatggaagtagtaagtaccatgcagactccgtgaa
gggccgattcaccatctccagagacaattcgaagaacacgctgtatctgcaaatgaacagcctgagagccgaggacacggctgtctattactgtgcg
agatgggggatgggtatagtagcctccccgggcaacgtactgaactactactactttatggacgtctggggccaagggaccacggtcatcgtctcct
ca
335S VH domain
(SEQ ID NO: 78)
EVQLVESGGGVVQPGRSLRLSCAASGFSFSNYAMHWVRQAPGKGLEWVAVTWYDGSSKYHADS
VKGRFTISRDNSKNTLYLQMNSLRAEDTAVYYCARWGMGIVASPGNVLNYYYFMDVWGQGTTVI
VSS
335S VL domain
(SEQ ID NO: 79)
tcctatgagctgacacagccaccctcggtgtcagtgtccccaggacagacggccaggatcacctgctctggaaatccattgccaaagaaacatactt
actggtaccagcagaagccaggccaggcccctgtgttaatgatatataaagacagtgagaggccctcacggatccctgagcgattctccggctccag
ttcagggacaacagtcacgttgaccatcagtggagtccaggcagaagacgaggctgactattactgtcaatcagcagacagcactggtattgtggtt
ttcggcggagggaccaagctgaccgtccta
335S VL domain
(SEQ ID NO: 80)
SYELTQPPSVSVSPGQTARITCSGNPLPKKHTYWYQQKPGQAPVLMIYKDSERPSRIPERFSGSSSGT
TVTLTISGVQAEDEADYYCQSADSTGIVVFGGGTKLTVL
285S VH domain
(SEQ ID NO: 81)
caggtccagctggtgcagtctggggctgaggtgaagaagcctgggtcctcggtgaaggtctcctgcaaggcttctggaggcaccttcagcacctatg
caatcacctgggtgcgacaggcccctggacaagggcttgagtggatgggaaggatcatccctatccttcgagtagcaaactacgcacagaagttcca
gggcagagtcacgattaccgcggacagctccacgagcacagcctacatggagctgagcagcctgagatctgaggacacggccgtgtattactgtgcg
aacctactagcatattgtggtggtgactgcagatctgactactggggccagggaaccctggtcaccgtctcctca
285S VH domain
(SEQ ID NO: 82)
QVQLVQSGAEVKKPGSSVKVSCKASGGTFSTYAITWVRQAPGQGLEWMGRIIPILRVANYAQKFQ
GRVTITADSSTSTAYMELSSLRSEDTAVYYCANLLAYCGGDCRSDYWGQGTLVTVSS
285S VL domain
(SEQ ID NO: 83)
gaaattgtgttgacgcagtctccaggcaccctgtctttgtctccaggggaaagagccaccctctcctgcagggccagtcagagtgttaagaacagct
acttagcctggtaccaccagaaacctggccaggctcccaggctcctcatctatggtgcatccagcagggccactggcatcccagacaggttcagtgg
cagtgggtctgggacagacttcactctcaccatcagcagactggagcctgaagattttgcggtgtattactgtcagcagtacggtagctcactttgg
acgttcggccaagggaccaaggtggatgtcaaa
285S VL domain
(SEQ ID NO: 84)
EIVLTQSPGTLSLSPGERATLSCRASQSVKNSYLAWYHQKPGQAPRLLIYGASSRATGIPDRFSGSGS
GTDFTLTISRLEPEDFAVYYCQQYGSSLWTFGQGTKVDVK
382S VH domain
(SEQ ID NO: 85)
caggtccagctggtgcagtctggggctgaggtgaagaagcctgggtcctcggtgaaggtctcctgcaaggcttctggaggcaccttcagcaactatg
gtatcagctgggtgcgacaggcccctggacaagggcttgagtggatgggaaggaacatccctatcattggtatagcaaactacgcacagaagttcca
ggacagagtcacgattaccgcggacaaatccacgagcacagtgttcatggagctgagaagcctgagatatgaggacacggccgtatattactgtgcg
agagaccctctagcagctcgccggggaaactggttcgacccctggggccagggaaccctggtcaccgtctcctca
382S VH domain
(SEQ ID NO: 86)
QVQLVQSGAEVKKPGSSVKVSCKASGGTFSNYGISWVRQAPGQGLEWMGRNIPIIGIANYAQKFQD
RVTITADKSTSTVFMELRSLRYEDTAVYYCARDPLAARRGNWFDPWGQGTLVTVSS
382S VL domain
(SEQ ID NO: 87)
gacatccagatgacccagtctccttccaccctgtctgcatctgtaggagacagagtcaccatcacttgccgggccagtcagagtgttagtagttggt
tggcctggtatcagcagaaaccagggaaagcccctaaactcctgatctatgatgcctccactttgcaaagtggggtcccatcaaggttcagcggcag
tggatctgggacagaattcactctcaccatcagcagcctgcagcctgatgatttcgcaacttattactgccaacagtataatagttattcgtacact
tttggccaggggaccaagctggagatcaaa
382S VL domain
(SEQ ID NO: 88)
DIQMTQSPSTLSASVGDRVTITCRASQSVSSWLAWYQQKPGKAPKLLIYDASTLQSGVPSRFSGSGS
GTEFTLTISSLQPDDFATYYCQQYNSYSYTFGQGTKLEIK
365S VH domain
(SEQ ID NO: 89)
gaagtgcagctggtggagtctgggggaggcttggtacagcctggagggtccctgagactctcctgtgcagcctctggactcactttcagtgcttatg
aaatgaactgggtccgccaggctccaggaaaggggctggagtgggtttcacacattactactagtggcagtgccatatactacgcagattctgtgaa
gggccgattcaccatctccagagacaacgccaagaactcactgcatctgcaaatgaacagactgagagccgacgacacggctgtatatttctgtgcg
agagatagtaccatcgttcaatgtagtggcaccagctgccatgtgtcCacctactcctacatggacgtctggggcaaagggaccacagtcaccgtct
cttca
365S VH domain
(SEQ ID NO: 90)
EVQLVESGGGLVQPGGSLRLSCAASGLTFSAYEMNWVRQAPGKGLEWVSHITTSGSAIYYADSVK
GRFTISRDNAKNSLHLQMNRLRADDTAVYFCARDSTIVQCSGTSCHVSTYSYMDVWGKGTTVTVS
S
365S VL domain
(SEQ ID NO: 91)
gaaattgtgttgacacagtctccagccaccctgtctttgtctccaggggaaagagccaccctctcctgcagggccagtcagagtgttaccacctact
tagcctggtaccaacacaaacctggccaggctcccaggctcctcatttatgatgcatccaagagggccactggcatcccagccaggttcagtggcag
tgggtctgggacagacttcactctcaccatcagcagcctagagcctgaagattttgcagtttatcactgtcagcagcgtagcagctggcctccgatc
accttcggccaagggacacgcctggagattaaa
365S VL domain
(SEQ ID NO: 92)
EIVLTQSPATLSLSPGERATLSCRASQSVTTYLAWYQHKPGQAPRLLIYDASKRATGIPARFSGSGSG
TDFTLTISSLEPEDFAVYHCQQRSSWPPITFGQGTRLEIK
405S VH domain
(SEQ ID NO: 93)
gaagtgcagctggtggagtctggaggaggcttgatccagcctggggggtccctgagactctcctgtgcagcctctgggttcaccgtcagtagccact
acatgagttgggtccgccaggctccagggaaggggctggagtgggtctcaggtatttatagcggtggtaggacacactacgcagactccgtgaaggg
ccgattcaccatctccagagacaattccaagaacacgctgtatcttcaaatgaacagcctgagagccgatgacacggccgtgtattcctgtgcgaga
gggaccctactttacttgtacggtatggacgtctggggccaagggaccacggtcaccgtctcctca
405S VH domain
(SEQ ID NO: 94)
EVQLVESGGGLIQPGGSLRLSCAASGFTVSSHYMSWVRQAPGKGLEWVSGIYSGGRTHYADSVKG
RFTISRDNSKNTLYLQMNSLRADDTAVYSCARGTLLYLYGMDVWGQGTTVTVSS
405S VL domain
(SEQ ID NO: 95)
cagtctgtgctgacgcagccgccctcagtgtctgcggccccagggcagagggtcaccatctcctgcactgggagcagctccaacatcggggcaggtt
ataatgtacactggtaccagcaacttccaggagcagcccccaaactcctcatctatggtaacaccaatcggccctcaggggtccctgaccgattctc
tggctccaagtctggcacctcagcctccctggccatcactgggctccaggctgaggatgaggctgattattactgccagtcctttgacagcagcctg
agtggggtcttcggaactgggaccaaggtcaccgtcctg
405S VL domain
(SEQ ID NO: 96)
QSVLTQPPSVSAAPGQRVTISCTGSSSNIGAGYNVHWYQQLPGAAPKLLIYGNTNRPSGVPDRFSGS
KSGTSASLAITGLQAEDEADYYCQSFDSSLSGVFGTGTKVTVL
102S VH domain
(SEQ ID NO: 97)
gaagtgcagctggtggagtctgggggaggcttggtacagcctggagggtccctgagactctcctgttcagcctctggattcaccttccacagttacg
acatgaactgggcAcgccaggctccagggaaggggctggagtgggtttcagagattagtagtagtgggcaaagcaaaaattacgcagactctgtgag
gggccgattcaccatctccagagacaacgccaagaactcactgtatctgcaaataaacagcctgcgaagcgaggacacggctgtttattactgtgcg
cgagtgggttttgtggccaagtttgggagtgatttttctatcactcacgggacgtactacttggactactggggccagggaaccctggtcaccgtct
cctca
102S VH domain
(SEQ ID NO: 98)
EVQLVESGGGLVQPGGSLRLSCSASGFTFHSYDMNWARQAPGKGLEWVSEISSSGQSKNYADSVR
GRFTISRDNAKNSLYLQINSLRSEDTAVYYCARVGFVAKFGSDFSITHGTYYLDYWGQGTLVTVSS
102S VL domain
(SEQ ID NO: 99)
caacttgtgttgacgcagtctccagccaccctgtctttgtctccaggggaaagagccaccctctcctgcagggccagtcagagtgttagcaactact
tagcctggtaccaacagaaacctggccaggctcccaggctcctcatctatgatgcatccaacagggcccctggcgttccagccaggttcagtggcag
tgggtctgggacagacttctctctcaccatcagcagccttgagcctgaagattttgcagtttattactgtctgcagcgtagcaactggcctccttgg
acgttcggccaagggaccaaggtggaaatcaaa
102S VL domain
(SEQ ID NO: 100)
QLVLTQSPATLSLSPGERATLSCRASQSVSNYLAWYQQKPGQAPRLLIYDASNRAPGVPARFSGSGS
GTDFSLTISSLEPEDFAVYYCLQRSNWPPWTFGQGTKVEIK
578S VH domain
(SEQ ID NO: 101)
gaggtgcagctggtggagagcggcggcggcgtggtgcagcccggcaggagcctgaggctgagctgcgccgccagcggcttcaccttcagcagctacg
agctgcactgggtgaggcaggcccccggcaagggcctggagtgggtggccgtgatcagctacgacggcagcagcaagtactacgccgacagcctgaag
ggcaggttcaccatcagcagggacaacagcaagaacaccctgagcctgcagatgaacagcctgagggccgaggacaccgccatgtactactgcgtgagg
cagagcgaggcctactgcagcgacggcaggtgcgactggaggagggccctggacatctggggccagggcaccatggtgaccgtgagcccc
578S VH domain
(SEQ ID NO: 102)
EVQLVESGGGVVQPGRSLRLSCAASGFTFSSYELHWVRQAPGKGLEWVAVISYDGSSKYYADSLK
GRFTISRDNSKNTLSLQMNSLRAEDTAMYYCVRQSEAYCSDGRCDWRRALDIWGQGTMVTVSP
578S VL domain
(SEQ ID NO: 103)
gacatccagatgacccagtctccatcctccctgtctgcatctgtaggagacagagtcaccatcacttgccaggcgagtcaggacattaagaactatt
taaattggtatcagcagaaaccagggaaagcccctaagctcctgatctacgatgtatccaaattggaaacaggggtcccatcaaggttcagtggaag
tggatctgggacagatttttctttcaccatcagcagcctgcagcctgaagatattgcaacatattactgtcaacaatatgataatctgccctcgtac
acttttggccaggggaccaagctggagatcaaa
578S VL domain
(SEQ ID NO: 104)
DIQMTQSPSSLSASVGDRVTITCQASQDIKNYLNWYQQKPGKAPKLLIYDVSKLETGVPSRFSGSGS
GTDFSFTISSLQPEDIATYYCQQYDNLPSYTFGQGTKLEIK
191L VH domain
(SEQ ID NO: 105)
gaggtgcagctggtggagagcggcggcggcctggtgcagcccggggcagcctgaggctgagctgcgccgccagcggcttcaccttcagcagctacg
gcatccactgggtgaggcaggcccccggcaagggcctggagtgggtggccatcatcagctacgacggcaacaagaagttccacgccgacagcgtgaa
ggacaggttcaccatcagcagggacaacagcaagaacatgctgtacctgcagatgaacaacctgaagctggaggacaccgccgtgtactactgcgcc
agggcctacgacagcggctacttcgccggcagcgtgttctactactggggccagggcgtgctggtgaccgtgagcagcg
191L VH domain
(SEQ ID NO: 106)
EVQLVESGGGLVQPGGSLRLSCAASGFTFSSYGIHWVRQAPGKGLEWVAIISYDGNKKFHADSVKD
RFTISRDNSKNMLYLQMNNLKLEDTAVYYCARAYDSGYFAGSVFYYWGQGVLVTVSS
191L VL domain
(SEQ ID NO: 107)
gaggtggtgttcacccagccccacagcgtgagcggcagccccggccagaccgtgaccatcagctgcaccaggagcagcggcagcatcgacagcgagt
acgtgcagtggtaccagcagaggcccggcaacgcccccaccaccgtgatctacaaggacaaccagaggcccagcggcgtgcccgacaggttcagcgg
cagcatcgacagcagcagcaacagcgccagcctggccatcagcggcctgaagagcgaggacgaggccgactactactgccagagcgccgacggcaac
taccaccccgtgttcggcgagggcaccaggctgaccgtgctgg
191L VL domain
(SEQ ID NO: 108)
EVVFTQPHSVSGSPGQTVTISCTRSSGSIDSEYVQWYQQRPGNAPTTVIYKDNQRPSGVPDRFSGSID
SSSNSASLAISGLKSEDEADYYCQSADGNYHPVFGEGTRLTVL
206L VH domain
(SEQ ID NO: 109)
gaggtgcagctggtggagagcggcggcggcgtggtgcagcccggcggcagcctgaggctgagctgcgccgccagcggcttcaccttcgacgactacg
ccatgcactgggtgaggcaggcccccggcaagggcctggagtgggtgagcggcatcagctggagcggcggcaacacctactacgccgacagcgtgaa
gggcaggttcaccgtgagcagggagaacgccaagaacagcctgtacctgcagatgaacaggctgagggccgaggacaccgccttctacaactgcgcc
aggaccagccagggcggcgccgtggtgttcagcgcccccaactactggggccagggcgtgctggtgaccgtgagcagcg
206L VH domain
(SEQ ID NO: 110)
EVQLVESGGGVVQPGGSLRLSCAASGFTFDDYAMHWVRQAPGKGLEWVSGISWSGGNTYYADSV
KGRFTVSRENAKNSLYLQMNRLRAEDTAFYNCARTSQGGAVVFSAPNYWGQGVLVTVSS
206L VL domain
(SEQ ID NO: 111)
cagagcgtgctgacccaggcccccagcgtgagcggcgcccccggccagagggtgaccatcagctgcaccggcagcagcagcaacatcggcggctact
acgtgcagtggtaccagcagctgcccggcagggcccccaagctgctgatctacgagaacaacaagaggcccagcggcgtgagcgacaggttcagcgg
cagcaggagcgccagcagcgccagcctgaccatcaccggcctgcagagcgaggacgaggccgactactactgccagagctacgacagcaggctgaac
ggctacatcttcggcggcggcaccaggctgaccgtgctgg
206L VL domain
(SEQ ID NO: 112)
QSVLTQAPSVSGAPGQRVTISCTGSSSNIGGYYVQWYQQLPGRAPKLLIYENNKRPSGVSDRFSGSR
SASSASLTITGLQSEDEADYYCQSYDSRLNGYIFGGGTRLTVL
231L VH domain
(SEQ ID NO: 113)
gaggtgcagctggtggagagcggcggcggcctggtgcagcccggcggcagcctgaggctgagctgcgccgccagcgccttcaccttcgacgactacg
ccatgcactgggtgaggcaggcccccggcaagggcctggagtgggtgagcggcatcacctggagcggcctgagcacctactacgccgacagcgtgaa
gggcaggttcaccatcagcagggacaacgccaagaacagcctgtacctgcagatgaacagcctgagggtggaggacaccgccctgtactactgcgcc
aagagcggcaacaacagccccttcagcggcctggacagctggggccagggcgccgtggtgaccgtgagcagcg
231L VH domain
(SEQ ID NO: 114)
EVQLVESGGGLVQPGGSLRLSCAASAFTFDDYAMHWVRQAPGKGLEWVSGITWSGLSTYYADSV
KGRFTISRDNAKNSLYLQMNSLRVEDTALYYCAKSGNNSPFSGLDSWGQGAVVTVSS
231L VL domain
(SEQ ID NO: 115)
cagagcgtgctgacccagccccccagcgtgagcggcgcccccggccagagggtgaccgtgagctgcaccggcagcagcagcaacatcggcgccggc
agctacgtgcagtggtaccagcagctgcccggcaccgcccccaagctgctgatctacgagaacaacaagaggcccagcggcgtgagcgacaggttca
gcggcagcaagagcggcaccagcgccagcctgaccatcaccggcctgcagagcgaggacgaggccgactactactgccagagctacgacagcagcct
gaacgcctacatcttcggcgccggcaccaggctgaccgtgctgg
231L VL domain
(SEQ ID NO: 116)
QSVLTQPPSVSGAPGQRVTVSCTGSSSNIGAGSYVQWYQQLPGTAPKLLIYENNKRPSGVSDRFSGS
KSGTSASLTITGLQSEDEADYYCQSYDSSLNAYIFGAGTRLTVL
232L VH domain
(SEQ ID NO: 117)
aagagccagctggtggagagcggcggcggcctggtgcagcccggcggcagcctgaggctgagctgcgccgccagcggcttcaccttcgacgcctacg
ccatgcactgggtgaggcaggcccccggcgagggcctggagtgggtgaccgccatcagctggagcggcagcagcacctactacgccgacagcgtgaa
gggcaggttcaccttcagcagggacaacgccaagaacagcctgtacctggagatgaacagcctgagggccgacgacaccgccttctactactgcgtg
aagggcggcgtgtacaactggttcgacgtgtggggccccggcgtgctggtgaccgtgagcagcg
232L VH domain
(SEQ ID NO: 118)
KSQLVESGGGLVQPGGSLRLSCAASGFTFDAYAMHWVRQAPGEGLEWVTAISWSGSSTYYADSV
KGRFTFSRDNAKNSLYLEMNSLRADDTAFYYCVKGGVYNWFDVWGPGVLVTVSS
232L VL domain
(SEQ ID NO: 119)
cagagcgtgctgacccagccccccagcgtgagcggcgcccccggccagagggtgaccatcagctgcaccggcagcaggagcaacatcggcggctact
acgtgagctggtaccagcagttccccggcaccacccccaagctgctgatctaccaggacaacaagaggcccagcggcgtgagcgacaggttcagcgg
cagcaagagcggcaccagcgccagcctgaccatcaccggcctgcagaccgaggacgaggccgactactactgcctgagctacgacagcagcctgaac
ggctgggtgttcggcggcggcaccaggctgaccgtgctgg
232L VL domain
(SEQ ID NO: 120)
QSVLTQPPSVSGAPGQRVTISCTGSRSNIGGYYVSWYQQFPGTTPKLLIYQDNKRPSGVSDRFSGSK
SGTSASLTITGLQTEDEADYYCLSYDSSLNGWVFGGGTRLTVL
310L VH domain
(SEQ ID NO: 121)
gagatgcagctggtggagagcggcggcggcctggtgcagcccggcggcagcctgaggctgagctgcgccgccagcggcttcaccttcgacgcctacg
ccatgcactgggtgaggcagaggcccggcaagggcctggagtggatcagcggcatcatctggagcggcaggaccacctactacgccgacagcgtgaa
gggcaggttcaccttcagcagggacaacagcaagaacagcctgtacctggagatgaacagcctgaagcccgaggacaccgccctgtactactgcgcc
aaggacagctggaactacggcgtgtacaacagcctggacgtgtggggcaggggcatcctggtgaccgtgagcagcg
310L VH domain
(SEQ ID NO: 122)
EMQLVESGGGLVQPGGSLRLSCAASGFTFDAYAMHWVRQRPGKGLEWISGIIWSGRTTYYADSVK
GRFTFSRDNSKNSLYLEMNSLKPEDTALYYCAKDSWNYGVYNSLDVWGRGILVTVSS
310L VL domain
(SEQ ID NO: 123)
cagagcgtgctgacccagccccccagcgtgagcggcgcccccggccagagggtgaccatcagctgcaccggcagcagcagcaacgtgggcccccact
acgtgagctggtaccagcacctgcccggcaccacccccaagctgctgatcttccaggacaacaagaggcccagcggcgtgagcgacaggttcagcgg
cagcaagagcgccagcagcgccagcctgaccatcaccggcctgcagaccgaggacgaggccgactactactgcctgagctacgacagcagcctgaac
gtgtacatcttcggcagcggcaccaggctgaccgtgctgg
310L VL domain
(SEQ ID NO: 124)
QSVLTQPPSVSGAPGQRVTISCTGSSSNVGPHYVSWYQHLPGTTPKLLIFQDNKRPSGVSDRFSGSK
SASSASLTITGLQTEDEADYYCLSYDSSLNVYIFGSGTRLTVL
314L VH domain
(SEQ ID NO: 125)
gaggagaggctggtggagagcggcggcggcctggtgcagcccggcggcagcctgaggctgagctgcgccgccagcggcttcaccttcggcgactacg
ccatgcactgggtgaggcaggtgcccggcaagagcctggagtggctgagcggcatcagctggagcggctacaccacctacagcgccgacagcgtgaa
gggcaggttcaccatcagcagggacaacgccaagaacagcctgtacctgcagatgaacaggctgaggcccgaggacaccggcctgtactactgcgtg
accctgatcggcgccatcggcatcaaggacagcctggacgtgtggggcaggggcgtgctggtgaccgtgagcagcg
314L VH domain
(SEQ ID NO: 126)
EERLVESGGGLVQPGGSLRLSCAASGFTFGDYAMHWVRQVPGKSLEWLSGISWSGYTTYSADSVK
GRFTISRDNAKNSLYLQMNRLRPEDTGLYYCVTLIGAIGIKDSLDVWGRGVLVTVSS
314L VL domain
(SEQ ID NO: 127)
gaggtggtgttcacccagccccacagcgtgagcggcagccccggccagatggtgaccatcagctgcaccaggagcagcggcaccatcgacaacagcta
cgtgtactggcaccagcagaggcccggcagcgcccccaccaccgtgatctacaacgacgaccagaggcccagcggcgtgcccgacaggttcagcggc
agcatcgacagcagcagcaacagcgccagcctgaccatcagcggcctgaagagcgaggacgaggccgactactactgccagagctacgacagcagcg
gctgggtgttcggcggcggcaccaggctgaccgtgctgg
314L VL domain
(SEQ ID NO: 128)
EVVFTQPHSVSGSPGQMVTISCTRSSGTIDNSYVYWHQQRPGSAPTTVIYNDDQRPSGVPDRFSGSI
DSSSNSASLTISGLKSEDEADYYCQSYDSSGWVFGGGTRLTVL
315L VH domain
(SEQ ID NO: 129)
caggtgcagctgcaggagagcggccccggcctggtgaagcccagcgagaccctgagcctgacctgcaccgtgagcggcggcagcatcaccggcaact
actggacctgggtgaggcagccccccggcaagggcctgcagtacatcggcaggttcgacgccggcggcaagacctactacaacccccccctgaagagc
agggtgagcatcctgggcgacaagagcaagaaccaggtgagcctgaacctgaccagcgtgaccgtggccgacaccggcgtgtactactgcgccaggga
gcagtggggccagctgctgagcggcagcttcgacgtgtggggcaggggcgtgctggtgaccgtgagcagcg
315L VH domain
(SEQ ID NO: 130)
QVQLQESGPGLVKPSETLSLTCTVSGGSITGNYWTWVRQPPGKGLQYIGRFDAGGKTYYNPPLKSR
VSILGDKSKNQVSLNLTSVTVADTGVYYCAREQWGQLLSGSFDVWGRGVLVTVSS
315L VL domain
(SEQ ID NO: 131)
agctacgagctgacccagccccccagcgtgagcgtgagccccggccagaccgccaggatcacctgcagcggcgacgccctgcccgactactacgccca
ctggtaccagcagaagagcggccaggcccccatgctggtgatctacaaggacaccgagaggcccagcggcatccccgagaggttcagcggcagcagca
gcggcaccaccgtgagcctgaccatcagcggcgtgcaggccgaggacgaggccgactactactgccagagcggcgacagcagcggcaaccactgggt
gttcggcggcggcaccaggctgaccgtgctgg
315L VL domain
(SEQ ID NO: 132)
SYELTQPPSVSVSPGQTARITCSGDALPDYYAHWYQQKSGQAPMLVIYKDTERPSGIPERFSGSSSG
TTVSLTISGVQAEDEADYYCQSGDSSGNHWVFGGGTRLTVL
396L VH domain
(SEQ ID NO: 133)
aagaggggctggtggagcctgggcgaggcctggagcagcctgggggcagcctgaggctgagctgcgccgccagcggcttcaacttcggcgactacgc
catgcactgggtgaggcaggtgcccggcaagagcctggagtggctgagcggcatcagctggagcggctacaccacctacagcgccgacagcgtgaagg
gcaggttcaccatcagcagggacaacgccaagaacagcctgtacctgcagatgaacaggctgaggcccgaggacaccggcctgtactactgcgtgac
cctgatcggcgccatcggcatcaaggacagcctggacgtgtggggcaggggcgtgctggtgaccgtgagcagcg
396L VH domain
(SEQ ID NO: 134)
KRGWWSLGEAWSSLGGSLRLSCAASGFNFGDYAMHWVRQVPGKSLEWLSGISWSGYTTYSADSV
KGRFTISRDNAKNSLYLQMNRLRPEDTGLYYCVTLIGAIGIKDSLDVWGRGVLVTVSS
396L VL domain
(SEQ ID NO: 135)
cagagcgtgctgacccagccccccagcgtgagcggcgcccccggccagagcatcaccgtgagctgcaccggcagcagcagcaacatcggcgccggca
acttcgtgcagtggtacaggcacctgcccggcaccgcccccaagctgctgatctaccagaccgacaagaggcccagcggcctgagcgacaggctgag
cggcagcaggagcggcagcagcgccagcctgaccatcaccggcctgcagagcgacgacgaggccgactactactgccaggcctacgacagccacagc
aacacctggctgttcggcggcggcaccaggctgaccgtgctgg
396L VL domain
(SEQ ID NO: 136)
QSVLTQPPSVSGAPGQSITVSCTGSSSNIGAGNFVQWYRHLPGTAPKLLIYQTDKRPSGLSDRLSGSR
SGSSASLTITGLQSDDEADYYCQAYDSHSNTWLFGGGTRLTVL

TABLE 18
Locations of the CDR sequences in each variable domain

| Antibody | SEQ ID | | | |
|---|---|---|---|---|
| Domain | NO: | CDR1 | CDR2 | CDR3 |
| 316L VH | 2 | 26-33 | 51-58 | 96-112 |
| 316L VL | 4 | 26-34 | 52-54 | 90-102 |
| 380L VH | 6 | 26-33 | 51-58 | 96-112 |
| 380L VL | 8 | 26-34 | 52-54 | 90-102 |
| 291S VH | 10 | 26-34 | 52-58 | 96-108 |
| 291S VL | 12 | 26-34 | 52-58 | 96-106 |
| 545S VH | 14 | 26-35 | 53-59 | 97-111 |
| 545S VL | 16 | 26-34 | 52-58 | 96-106 |
| 523S VH | 18 | 26-33 | 51-58 | 96-114 |
| 523S VL | 20 | 26-33 | 51-53 | 89-101 |
| 573S VH | 22 | 26-33 | 51-58 | 96-116 |
| 573S VL | 24 | 27-32 | 50-52 | 88-99 |
| 541S VH | 26 | 26-33 | 51-58 | 96-112 |
| 541S VL | 28 | 26-33 | 51-53 | 89-100 |
| 294S VH | 30 | 26-35 | 53-59 | 97-115 |
| 294S VL | 32 | 27-32 | 50-52 | 88-99 |
| 241S VH | 34 | 26-33 | 51-58 | 96-115 |
| 241S VL | 36 | 26-34 | 52-54 | 90-101 |
| 354S VH | 38 | 26-33 | 51-58 | 96-114 |
| 354S VL | 40 | 27-32 | 50-52 | 88-102 |
| 233S VH | 42 | 26-33 | 51-58 | 96-114 |
| 233S VL | 44 | 27-32 | 50-52 | 88-98 |
| 503S VH | 46 | 26-33 | 51-58 | 96-116 |
| 503S VL | 48 | 27-32 | 50-52 | 88-98 |
| 203S VH | 50 | 26-33 | 51-58 | 96-114 |
| 203S VL | 52 | 27-32 | 50-52 | 88-98 |
| 315S VH | 54 | 26-33 | 51-58 | 96-111 |
| 315S VL | 56 | 27-32 | 50-52 | 88-98 |
| 586S VH | 58 | 26-33 | 51-58 | 96-110 |
| 586S VL | 60 | 27-32 | 50-52 | 88-97 |
| 377S VH | 62 | 26-33 | 51-58 | 96-110 |
| 377S VL | 64 | 27-33 | 51-53 | 89-98 |
| 528S VH | 66 | 26-33 | 51-58 | 96-121 |
| 528S VL | 68 | 27-32 | 50-52 | 88-99 |
| 246S VH | 70 | 26-33 | 51-58 | 96-112 |
| 246S VL | 72 | 27-33 | 51-53 | 89-99 |
| 338S VH | 74 | 26-33 | 51-58 | 96-119 |
| 338S VL | 76 | 27-32 | 50-52 | 88-98 |
| 335S VH | 78 | 26-33 | 51-58 | 96-120 |
| 335S VL | 80 | 26-31 | 49-51 | 87-98 |
| 285S VH | 82 | 26-33 | 51-58 | 96-112 |
| 285S VL | 84 | 27-33 | 51-53 | 89-99 |
| 382S VH | 86 | 26-33 | 51-58 | 96-112 |
| 382S VL | 88 | 27-32 | 50-52 | 88-98 |
| 365S VH | 90 | 26-33 | 51-58 | 96-121 |
| 365S VL | 92 | 27-32 | 50-52 | 88-99 |
| 405S VH | 94 | 26-33 | 51-57 | 95-109 |
| 405S VL | 96 | 26-34 | 52-54 | 90-101 |
| 102S VH | 98 | 26-33 | 51-58 | 96-121 |
| 102S VL | 100 | 27-32 | 50-52 | 88-99 |
| 578S VH | 102 | 26-33 | 51-58 | 97-117 |
| 578S VL | 104 | 27-32 | 50-52 | 89-98 |
| 191L VH | 106 | 26-33 | 51-58 | 97-112 |
| 191L VL | 108 | 26-33 | 51-53 | 92-101 |
| 206L VH | 110 | 26-33 | 51-58 | 97-112 |
| 206L VL | 112 | 26-33 | 51-53 | 90-100 |
| 231L VH | 114 | 26-33 | 51-58 | 97-110 |
| 231L VL | 116 | 26-34 | 52-54 | 91-101 |
| 232L VH | 118 | 26-33 | 51-58 | 97-107 |
| 232L VL | 120 | 26-33 | 51-53 | 90-100 |
| 310L VH | 122 | 26-33 | 51-58 | 97-111 |
| 310L VL | 124 | 26-33 | 51-53 | 90-100 |
| 314L VH | 126 | 26-33 | 51-58 | 97-111 |
| 314L VL | 128 | 26-33 | 51-53 | 92-100 |
| 315L VH | 130 | 26-33 | 51-57 | 96-110 |
| 315L VL | 132 | 26-31 | 49-51 | 88-98 |
| 396L VH | 134 | 26-33 | 51-58 | 97-111 |
| 396L VL | 136 | 26-34 | 52-54 | 91-101 |

The VH and VL (lambda) regions of the macaque-derived antibodies were cloned into a macaque IgG1 heavy chain vector and a lambda chain vector, respectively. As an example, the heavy and light chain amino acid sequences of the 316L antibody are shown below. The leader sequence is indicated by italics and the underlined portion corresponds to the constant region of the light chain or heavy chain. The VH or VL domain is shown in normal font.

316L macaque heavy chain (IgG1) amino acid
sequence
(SEQ ID NO: 137)
MGWSCIILFLVATATGVHSEERLVESGGGLVQPGGSLRLSCAASGFTFGD
YAMHWVRQVPGKSLEWLSGISWSGYTTYSADSVKGRFTISRDNAKNSLYL
QMNRLRPEDTGLYYCVTLIGAIGIKDSLDVWGRGVLVTVSSASTKGPSVF
PLAPSSRSTSESTAALGCLVKDYFPEPVTVSWNSGSLTSGVHTFPAVLQS
SGLYSLSSVVTVPSSSLGTQTYVCNVNHKPSNTKVDKRVEIKTCGGGSKP
PTCPPCTSPELLGGPSVFLFPPKPKDTLMISRTPEVTCVVVDVSQEDPDV
KFNWYVNGAEVHHAQTKPRETQYNSTYRVVSVLTVTHQDWLNGKEYTCKV
SNKALPAPIQKTISKDKGQPREPQVYTLPPSREELTKNQVSLTCLVKGFY
PSDIVVEWESSGQPENTYKTTPPVLDSDGSYFLYSKLTVDKSRWQQGNVF
SCSVMHEALHNHYTQKSLSLSPGK
316L macaque lambda chain amino acid sequence
(SEQ ID NO: 138)
MGWSCIILFLVATATGVHSQSVLTQPPSVSGAPGQRVTVSCSGSSSNIGA
GNYVQWYQQLPGTAPKVLIYQTEKRPSGTSDRFSGSKSDTSASLTINGLQ
SEDEADYYCQVYDSNLNGWVFGGGTRLTVLGQPKAAPSVTLFPPSSEELQ
ANKATLVCLISDFYPGAVEVAWKADGSAVNAGVETTKPSKQSNNKYAASS
YLSLTSDQWKSHKSYSCQVTHEGSTVEKTVAPAECS

The human-derived antibodies with lambda light chains were cloned into a human IgG1 heavy chain vector and a lambda chain vector, respectively. As examples, the amino acid sequences of the heavy and light chain of antibodies 545S and 523S are shown below. The leader sequence is indicated by italics and the underlined portion corresponds to the constant region of the light chain or heavy chain. The VH or VL domain is shown in normal font.

545S human heavy chain (IgG1) amino acid sequence
(SEQ ID NO: 139)
MGWSCIILFLVATATGVHSQVQLQESGPGLVKPSQTLSLTCTVSGGSISR
AGSYWSWIRQHPGKGLEWIGYISYSGSTYYNPSLESRVTMSLDTSKSQFS
LKLSSVTAADTAVYYCAREDRSGFYGLDVWGQGTTVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPGK
545S human lambda chain amino acid sequence
(SEQ ID NO: 140)
MGWSCIILFLVATATGSVTQSVLTQPSSLSASPGATASLTCTLRSGINVD
TYRIYWYQQKPGSPPQYLLRYKSDSDKHQGSGVPSRFSGSKDASANAGIL
LISGLQSADEADYYCMIWHSGAWVFGGGTKLTVQGQPKAAPSVTLFPPSS
EELQANKATLVCLISDFYPGAVTVAWKADSSPVKAGVETTTPSKQSNNKY
AASSYLSLTPEQWKSHRSYSCQVTHEGSTVEKTVAPTECS
523S human heavy chain (IgG1) amino acid sequence
(SEQ ID NO: 141)
MGWSCIILFLVATATGVHSQVQLVQSGAELKKPGASVKVSCKASGYTFSD
HYVHWVRQAPGQGLEWMGRINPNSGGTNYAQRFLGRVTMTRDASISTAYL
DLSSLRSDDTAVYYCARSSSSWSGHYYYYMDVWGKGTTVTVSSASTKGPS
VFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFS
CSVMHEALHNHYTQKSLSLSPGK
523S human lambda chain amino acid sequence
(SEQ ID NO: 142)
MGWSCIILFLVATATGSVTQSVLTQPPSVSAAPGQRVTISCSGSTSNIAS
NFVSWYQQLPGTAPKLLIYDNHKRPSGIPDRFSGSKSGTSATLGITGLQT
GDEADYYCGTWDSSLSAGVFGGGTKVTVLGQPKAAPSVTLFPPSSEELQA
NKATLVCLISDFYPGAVTVAWKADSSPVKAGVETTTPSKQSNNKYAASSY
LSLTPEQWKSHRSYSCQVTHEGSTVEKTVAPTECS

The human-derived antibodies with kappa light chains were cloned into a human IgG1 heavy chain vector and a kappa chain vector, respectively. As an example, the amino acid sequences of the 294S heavy chain and light chain are shown below. The leader sequence is indicated by italics and the underlined portion corresponds to the constant region of the light chain or heavy chain. The VH or VL domain is shown in normal font.

294S human heavy chain (IgG1) amino acid sequence
(SEQ ID NO: 143)
MGWSCIILFLVATATGVHSQVQLQESGPGLVKPSQTLSLTCTVSGGSISS
GGYHWSWIRQHPGKGLEWIGNIYYNGRTYYNPSLKSRVTISVDTSENEFS
LKLSSVTAADTAVYYCARDRRRPRIGHDYGMDVWGQGTKVIVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEV
KENWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVF
SCSVMHEALHNHYTQKSLSLSPGK
294S human kappa chain amino acid sequence
(SEQ ID NO: 144)
MGWSCIILFLVATATGVHSDIQMTQSPSSLSASVGDRVTISCRASQSISN
FLNWYQQKPGQAPKLLIYTASNLQSGVPSRFSGSGSGTDFTLTISSLQPE
DFATYYCQQIYNTGMYSFGQGTKLEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC

The individual components encoded by the heavy chain and light chain vectors described above are provided below.

Leader sequences
(SEQ ID NO: 145)
MGWSCIILFLVATATGVHS
(SEQ ID NO: 146)
MGWSCIILFLVATATGSVT
Macaque heavy chain (IgG1) constant region
(SEQ ID NO: 147)
ASTKGPSVFPLAPSSRSTSESTAALGCLVKDYFPEPVTVSWNSGSLTSGV
HTFPAVLQSSGLYSLSSVVTVPSSSLGTQTYVCNVNHKPSNTKVDKRVEI
KTCGGGSKPPTCPPCTSPELLGGPSVFLFPPKPKDTLMISRTPEVTCVVV
DVSQEDPDVKFNWYVNGAEVHHAQTKPRETQYNSTYRVVSVLTVTHQDWL
NGKEYTCKVSNKALPAPIQKTISKDKGQPREPQVYTLPPSREELTKNQVS
LTCLVKGFYPSDIVVEWESSGQPENTYKTTPPVLDSDGSYFLYSKLTVDK
SRWQQGNVFSCSVMHEALHNHYTQKSLSLSPGK
Macaque lambda light chain constant region
(SEQ ID NO: 148)
GQPKAAPSVTLFPPSSEELQANKATLVCLISDFYPGAVEVAWKADGSAVN
AGVETTKPSKQSNNKYAASSYLSLTSDQWKSHKSYSCQVTHEGSTVEKTV
APAECS
Human heavy chain (IgG1) constant region
(SEQ ID NO: 149)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human lambda light chain constant region
(SEQ ID NO: 150)
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS
Human kappa light chain constant region
(SEQ ID NO: 151)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

### B. Monoclonal Antibodies

Provided herein are monoclonal antibodies that specifically bind GP from SUDV and/or EBOV. In some aspects, the GP-specific monoclonal antibodies include a VH domain and/or a VL domain, and have the VH domain and/or VL domain complementarity determining region (CDR) sequences of any one of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L and 396L antibodies.

In some aspects, the monoclonal antibody includes the VH domain CDR1, CDR2 and CDR3 sequences of SEQ ID NO: 2 and the VL domain CDR1, CDR2 and CDR3 sequences of SEQ ID NO: 4. In some examples, the VH domain CDR1, CDR2 and CDR3 sequences respectively comprise residues 26-33, 51-58, and 96-112 of SEQ ID NO: 2, and the VL domain CDR1, CDR2 and CDR3 sequences respectively comprise residues 26-34, 52-54, and 90-102 of SEQ ID NO: 4. In specific examples, the amino acid sequence of the VH domain is at least 85%, at least 90%, at least 95%, at least 96%, at least 97%, at least 98% or at least 99% identical to SEQ ID NO: 2, and the amino acid sequence of the VL domain is at least 85%, at least 90%, at least 95%, at least 96%, at least 97%, at least 98% or at least 99% identical to SEQ ID NO: 4. In particular non-limiting examples, the amino acid sequence of the VH domain comprises SEQ ID NO: 2, and the amino acid sequence of the VL domain comprises SEQ ID NO: 4.

In some aspects, the monoclonal antibody includes the VH domain CDR1, CDR2 and CDR3 sequences of SEQ ID NO: 6, and the VL domain CDR1, CDR2 and CDR3 sequences of SEQ ID NO: 8. In some examples, the VH domain CDR1, CDR2 and CDR3 sequences respectively comprise residues 26-33, 51-58, and 96-112 of SEQ ID NO: 6, and the VL domain CDR1, CDR2 and CDR3 sequences respectively comprise residues 26-34, 52-54, and 90-102 of SEQ ID NO: 8. In specific examples, the amino acid sequence of the VH domain is at least 85%, at least 90%, at least 95%, at least 96%, at least 97%, at least 98% or at least 99% identical to SEQ ID NO: 6, and the amino acid sequence of the VL domain is at least 85%, at least 90%, at least 95%, at least 96%, at least 97%, at least 98% or at least 99% identical to SEQ ID NO: 8. In particular non-limiting examples, the amino acid sequence of the VH domain comprises SEQ ID NO: 6, and the amino acid sequence of the VL domain comprises SEQ ID NO: 8.

In some aspects, the monoclonal antibody includes the VH domain CDR1, CDR2 and CDR3 sequences of SEQ ID NO: 10, and the VL domain CDR1, CDR2 and CDR3 sequences of SEQ ID NO: 12. In some examples, the VH domain CDR1, CDR2 and CDR3 sequences respectively comprise residues 26-34, 52-58, and 96-108 of SEQ ID NO: 10, and the VL domain CDR1, CDR2 and CDR3 sequences respectively comprise residues 26-34, 52-58, and 96-106 of SEQ ID NO: 12. In specific examples, the amino acid sequence of the VH domain is at least 85%, at least 90%, at least 95%, at least 96%, at least 97%, at least 98% or at least 99% identical to SEQ ID NO: 10, and the amino acid sequence of the VL domain is at least 85%, at least 90%, at least 95%, at least 96%, at least 97%, at least 98% or at least 99% identical to SEQ ID NO: 12. In particular non-limiting examples, the amino acid sequence of the VH domain comprises SEQ ID NO: 10, and the amino acid sequence of the VL domain comprises SEQ ID NO: 12.

In some aspects, the monoclonal antibody includes the VH domain CDR1, CDR2 and CDR3 sequences of SEQ ID NO: 14, and the VL domain CDR1, CDR2 and CDR3 sequences of SEQ ID NO: 16. In some examples, the VH domain CDR1, CDR2 and CDR3 sequences respectively comprise residues 26-35, 53-59, and 97-111 of SEQ ID NO: 14, and the VL domain CDR1, CDR2 and CDR3 sequences respectively comprise residues 26-34, 52-58, and 96-106 of SEQ ID NO: 16. In specific examples, the amino acid sequence of the VH domain is at least 85%, at least 90%, at least 95%, at least 96%, at least 97%, at least 98% or at least 99% identical to SEQ ID NO: 14, and the amino acid sequence of the VL domain is at least 85%, at least 90%, at least 95%, at least 96%, at least 97%, at least 98% or at least 99% identical to SEQ ID NO: 16. In particular non-limiting examples, the amino acid sequence of the VH domain comprises SEQ ID NO: 14, and the amino acid sequence of the VL domain comprises SEQ ID NO: 16.

In some aspects, the monoclonal antibody includes the VH domain CDR1, CDR2 and CDR3 sequences of SEQ ID NO: 18, and the VL domain CDR1, CDR2 and CDR3 sequences of SEQ ID NO: 20. In some examples, the VH domain CDR1, CDR2 and CDR3 sequences respectively comprise residues 26-33, 51-58, and 96-114 of SEQ ID NO: 18, and the VL domain CDR1, CDR2 and CDR3 sequences respectively comprise residues 26-33, 51-53, and 89-101 of SEQ ID NO: 20. In specific examples, the amino acid sequence of the VH domain is at least 85%, at least 90%, at least 95%, at least 96%, at least 97%, at least 98% or at least 99% identical to SEQ ID NO: 18, and the amino acid sequence of the VL domain is at least 85%, at least 90%, at least 95%, at least 96%, at least 97%, at least 98% or at least 99% identical to SEQ ID NO: 20. In particular non-limiting examples, the amino acid sequence of the VH domain comprises SEQ ID NO: 18, and the amino acid sequence of the VL domain comprises SEQ ID NO: 20.

In some aspects, the monoclonal antibody includes the VH domain CDR1, CDR2 and CDR3 sequences of SEQ ID NO: 22, and the VL domain CDR1, CDR2 and CDR3 sequences of SEQ ID NO: 24. In some examples, the VH domain CDR1, CDR2 and CDR3 sequences respectively comprise residues 26-33, 51-58, and 96-116 of SEQ ID NO: 22, and the VL domain CDR1, CDR2 and CDR3 sequences respectively comprise residues 27-32, 50-52, and 88-99 of SEQ ID NO: 24. In specific examples, the amino acid sequence of the VH domain is at least 85%, at least 90%, at least 95%, at least 96%, at least 97%, at least 98% or at least 99% identical to SEQ ID NO: 22, and the amino acid sequence of the VL domain is at least 85%, at least 90%, at least 95%, at least 96%, at least 97%, at least 98% or at least 99% identical to SEQ ID NO: 24. In particular non-limiting examples, the amino acid sequence of the VH domain comprises SEQ ID NO: 22, and the amino acid sequence of the VL domain comprises SEQ ID NO: 24.

In some aspects, the monoclonal antibody includes the VH domain CDR1, CDR2 and CDR3 sequences of SEQ ID NO: 26, and the VL domain CDR1, CDR2 and CDR3 sequences of SEQ ID NO: 28. In some examples, the VH domain CDR1, CDR2 and CDR3 sequences respectively comprise residues 26-33, 51-58, and 96-112 of SEQ ID NO: 26, and the VL domain CDR1, CDR2 and CDR3 sequences respectively comprise residues 26-33, 51-53, and 89-100 of SEQ ID NO: 28. In specific examples, In specific examples, the amino acid sequence of the VH domain is at least 85%, at least 90%, at least 95%, at least 96%, at least 97%, at least 98% or at least 99% identical to SEQ ID NO: 26, and the amino acid sequence of the VL domain is at least 85%, at least 90%, at least 95%, at least 96%, at least 97%, at least 98% or at least 99% identical to SEQ ID NO: 28. In particular non-limiting examples, the amino acid sequence of the VH domain comprises SEQ ID NO: 26, and the amino acid sequence of the VL domain comprises SEQ ID NO: 28.

In some aspects, the monoclonal antibody includes the VH domain CDR1, CDR2 and CDR3 sequences of SEQ ID NO: 30, and the VL domain CDR1, CDR2 and CDR3 sequences of SEQ ID NO: 32. In some examples, the VH domain CDR1, CDR2 and CDR3 sequences respectively comprise residues 26-35, 53-59, and 97-115 of SEQ ID NO: 30, and the VL domain CDR1, CDR2 and CDR3 sequences respectively comprise residues 27-32, 50-52, and 88-99 of SEQ ID NO: 32. In specific examples, the amino acid sequence of the VH domain is at least 85%, at least 90%, at least 95%, at least 96%, at least 97%, at least 98% or at least 99% identical to SEQ ID NO: 30, and the amino acid sequence of the VL domain is at least 85%, at least 90%, at least 95%, at least 96%, at least 97%, at least 98% or at least 99% identical to SEQ ID NO: 32. In particular non-limiting examples, the amino acid sequence of the VH domain comprises SEQ ID NO: 30, and the amino acid sequence of the VL domain comprises SEQ ID NO: 32.

In some aspects, the monoclonal antibody includes the VH domain CDR1, CDR2 and CDR3 sequences of SEQ ID NO: 34, and the VL domain CDR1, CDR2 and CDR3 sequences of SEQ ID NO: 36. In some examples, the VH domain CDR1, CDR2 and CDR3 sequences respectively comprise residues 26-33, 51-58, and 96-115 of SEQ ID NO: 34, and the VL domain CDR1, CDR2 and CDR3 sequences respectively comprise residues 26-34, 52-54, and 90-101 of SEQ ID NO: 36. In specific examples, the amino acid sequence of the VH domain is at least 85%, at least 90%, at least 95%, at least 96%, at least 97%, at least 98% or at least 99% identical to SEQ ID NO: 34, and the amino acid sequence of the VL domain is at least 85%, at least 90%, at least 95%, at least 96%, at least 97%, at least 98% or at least 99% identical to SEQ ID NO: 36. In particular non-limiting examples, the amino acid sequence of the VH domain comprises SEQ ID NO: 34, and the amino acid sequence of the VL domain comprises SEQ ID NO: 36.

In some aspects, the monoclonal antibody includes the VH domain CDR1, CDR2 and CDR3 sequences of SEQ ID NO: 38, and the VL domain CDR1, CDR2 and CDR3 sequences of SEQ ID NO: 40. In some examples, the VH domain CDR1, CDR2 and CDR3 sequences respectively comprise residues 26-33, 51-58, and 96-114 of SEQ ID NO: 38, and the VL domain CDR1, CDR2 and CDR3 sequences respectively comprise residues 27-32, 50-52, and 88-102 of SEQ ID NO: 40. In specific examples, the amino acid sequence of the VH domain is at least 85%, at least 90%, at least 95%, at least 96%, at least 97%, at least 98% or at least 99% identical to SEQ ID NO: 38, and the amino acid sequence of the VL domain is at least 85%, at least 90%, at least 95%, at least 96%, at least 97%, at least 98% or at least 99% identical to SEQ ID NO: 40. In particular non-limiting examples, the amino acid sequence of the VH domain comprises SEQ ID NO: 38, and the amino acid sequence of the VL domain comprises SEQ ID NO: 40.

In some aspects, the monoclonal antibody includes the VH domain CDR1, CDR2 and CDR3 sequences of SEQ ID NO: 42, and the VL domain CDR1, CDR2 and CDR3 sequences of SEQ ID NO: 44. In some examples, the VH domain CDR1, CDR2 and CDR3 sequences respectively comprise residues 26-33, 51-58, and 96-114 of SEQ ID NO: 42, and the VL domain CDR1, CDR2 and CDR3 sequences respectively comprise residues 27-32, 50-52, and 88-98 of SEQ ID NO: 44. In specific examples, the amino acid sequence of the VH domain is at least 85%, at least 90%, at least 95%, at least 96%, at least 97%, at least 98% or at least 99% identical to SEQ ID NO: 42, and the amino acid sequence of the VL domain is at least 85%, at least 90%, at least 95%, at least 96%, at least 97%, at least 98% or at least 99% identical to SEQ ID NO: 44. In particular non-limiting examples, the amino acid sequence of the VH domain comprises SEQ ID NO: 42, and the amino acid sequence of the VL domain comprises SEQ ID NO: 44.

In some aspects, the monoclonal antibody includes the VH domain CDR1, CDR2 and CDR3 sequences of SEQ ID NO: 46, and the VL domain CDR1, CDR2 and CDR3 sequences of SEQ ID NO: 48. In some examples, the VH domain CDR1, CDR2 and CDR3 sequences respectively comprise residues 26-33, 51-58, and 96-116 of SEQ ID NO: 46, and the VL domain CDR1, CDR2 and CDR3 sequences respectively comprise residues 27-32, 50-52, and 88-98 of SEQ ID NO: 48. In specific examples, the amino acid sequence of the VH domain is at least 85%, at least 90%, at least 95%, at least 96%, at least 97%, at least 98% or at least 99% identical to SEQ ID NO: 46, and the amino acid sequence of the VL domain is at least 85%, at least 90%, at least 95%, at least 96%, at least 97%, at least 98% or at least 99% identical to SEQ ID NO: 48. In particular non-limiting examples, the amino acid sequence of the VH domain comprises SEQ ID NO: 46, and the amino acid sequence of the VL domain comprises SEQ ID NO: 48.

In some aspects, the monoclonal antibody includes the VH domain CDR1, CDR2 and CDR3 sequences of SEQ ID NO: 50, and the VL domain CDR1, CDR2 and CDR3 sequences of SEQ ID NO: 52. In some examples, the VH domain CDR1, CDR2 and CDR3 sequences respectively comprise residues 26-33, 51-58, and 96-114 of SEQ ID NO: 50, and the VL domain CDR1, CDR2 and CDR3 sequences respectively comprise residues 27-32, 50-52, and 88-98 of SEQ ID NO: 52. In specific examples, the amino acid sequence of the VH domain is at least 85%, at least 90%, at least 95%, at least 96%, at least 97%, at least 98% or at least 99% identical to SEQ ID NO: 50, and the amino acid sequence of the VL domain is at least 85%, at least 90%, at least 95%, at least 96%, at least 97%, at least 98% or at least 99% identical to SEQ ID NO: 52. In particular non-limiting examples, the amino acid sequence of the VH domain comprises SEQ ID NO: 50, and the amino acid sequence of the VL domain comprises SEQ ID NO: 52.

In some aspects, the monoclonal antibody includes the VH domain CDR1, CDR2 and CDR3 sequences of SEQ ID NO: 54, and the VL domain CDR1, CDR2 and CDR3 sequences of SEQ ID NO: 56. In some examples, the VH domain CDR1, CDR2 and CDR3 sequences respectively comprise residues 26-33, 51-58, and 96-111 of SEQ ID NO: 54, and the VL domain CDR1, CDR2 and CDR3 sequences respectively comprise residues 27-32, 50-52, and 88-98 of SEQ ID NO: 56. In specific examples, the amino acid sequence of the VH domain is at least 85%, at least 90%, at least 95%, at least 96%, at least 97%, at least 98% or at least 99% identical to SEQ ID NO: 54, and the amino acid sequence of the VL domain is at least 85%, at least 90%, at least 95%, at least 96%, at least 97%, at least 98% or at least 99% identical to SEQ ID NO: 56. In particular non-limiting examples, the amino acid sequence of the VH domain comprises SEQ ID NO: 54, and the amino acid sequence of the VL domain comprises SEQ ID NO: 56.

In some aspects, the monoclonal antibody includes the VH domain CDR1, CDR2 and CDR3 sequences of SEQ ID NO: 58, and the VL domain CDR1, CDR2 and CDR3 sequences of SEQ ID NO: 60. In some examples, the VH domain CDR1, CDR2 and CDR3 sequences respectively comprise residues 26-33, 51-58, and 96-110 of SEQ ID NO: 58, and the VL domain CDR1, CDR2 and CDR3 sequences respectively comprise residues 27-32, 50-52, and 88-97 of SEQ ID NO: 60. In specific examples, the amino acid sequence of the VH domain is at least 85%, at least 90%, at least 95%, at least 96%, at least 97%, at least 98% or at least 99% identical to SEQ ID NO: 58, and the amino acid sequence of the VL domain is at least 85%, at least 90%, at least 95%, at least 96%, at least 97%, at least 98% or at least 99% identical to SEQ ID NO: 60. In particular non-limiting examples, the amino acid sequence of the VH domain comprises SEQ ID NO: 58, and the amino acid sequence of the VL domain comprises SEQ ID NO: 60.

In some aspects, the monoclonal antibody includes the VH domain CDR1, CDR2 and CDR3 sequences of SEQ ID NO: 62, and the VL domain CDR1, CDR2 and CDR3 sequences of SEQ ID NO: 64. In some examples, the VH domain CDR1, CDR2 and CDR3 sequences respectively comprise residues 26-33, 51-58, and 96-110 of SEQ ID NO: 62, and the VL domain CDR1, CDR2 and CDR3 sequences respectively comprise residues 27-33, 51-53, and 89-98 of SEQ ID NO: 64. In specific examples, the amino acid sequence of the VH domain is at least 85%, at least 90%, at least 95%, at least 96%, at least 97%, at least 98% or at least 99% identical to SEQ ID NO: 62, and the amino acid sequence of the VL domain is at least 85%, at least 90%, at least 95%, at least 96%, at least 97%, at least 98% or at least 99% identical to SEQ ID NO: 64. In particular non-limiting examples, the amino acid sequence of the VH domain comprises SEQ ID NO: 62, and the amino acid sequence of the VL domain comprises SEQ ID NO: 64.

In some aspects, the monoclonal antibody includes the VH domain CDR1, CDR2 and CDR3 sequences of SEQ ID NO: 66, and the VL domain CDR1, CDR2 and CDR3 sequences of SEQ ID NO: 68. In some examples, the VH domain CDR1, CDR2 and CDR3 sequences respectively comprise residues 26-33, 51-58, and 96-121 of SEQ ID NO: 66, and the VL domain CDR1, CDR2 and CDR3 sequences respectively comprise residues 27-32, 50-52, and 88-99 of SEQ ID NO: 68. In specific examples, the amino acid sequence of the VH domain is at least 85%, at least 90%, at least 95%, at least 96%, at least 97%, at least 98% or at least 99% identical to SEQ ID NO: 66, and the amino acid sequence of the VL domain is at least 85%, at least 90%, at least 95%, at least 96%, at least 97%, at least 98% or at least 99% identical to SEQ ID NO: 68. In particular non-limiting examples, the amino acid sequence of the VH domain comprises SEQ ID NO: 66, and the amino acid sequence of the VL domain comprises SEQ ID NO: 68.

In some aspects, the monoclonal antibody includes the VH domain CDR1, CDR2 and CDR3 sequences of SEQ ID NO: 70, and the VL domain CDR1, CDR2 and CDR3 sequences of SEQ ID NO: 72. In some examples, the VH domain CDR1, CDR2 and CDR3 sequences respectively comprise residues 26-33, 51-58, and 96-112 of SEQ ID NO: 70, and the VL domain CDR1, CDR2 and CDR3 sequences respectively comprise residues 27-33, 51-53, and 89-99 of SEQ ID NO: 72. In specific examples, the amino acid sequence of the VH domain is at least 85%, at least 90%, at least 95%, at least 96%, at least 97%, at least 98% or at least 99% identical to SEQ ID NO: 70, and the amino acid sequence of the VL domain is at least 85%, at least 90%, at least 95%, at least 96%, at least 97%, at least 98% or at least 99% identical to SEQ ID NO: 72. In particular non-limiting examples, the amino acid sequence of the VH domain comprises SEQ ID NO: 70, and the amino acid sequence of the VL domain comprises SEQ ID NO: 72.

In some aspects, the monoclonal antibody includes the VH domain CDR1, CDR2 and CDR3 sequences of SEQ ID NO: 74, and the VL domain CDR1, CDR2 and CDR3 sequences of SEQ ID NO: 76. In some examples, the VH domain CDR1, CDR2 and CDR3 sequences respectively comprise residues 26-33, 51-58, and 96-119 of SEQ ID NO: 74, and the VL domain CDR1, CDR2 and CDR3 sequences respectively comprise residues 27-32, 50-52, and 88-98 of SEQ ID NO: 76. In specific examples, the amino acid sequence of the VH domain is at least 85%, at least 90%, at least 95%, at least 96%, at least 97%, at least 98% or at least 99% identical to SEQ ID NO: 74, and the amino acid sequence of the VL domain is at least 85%, at least 90%, at least 95%, at least 96%, at least 97%, at least 98% or at least 99% identical to SEQ ID NO: 76. In particular non-limiting examples, the amino acid sequence of the VH domain comprises SEQ ID NO: 74, and the amino acid sequence of the VL domain comprises SEQ ID NO: 76.

In some aspects, the monoclonal antibody includes the VH domain CDR1, CDR2 and CDR3 sequences of SEQ ID NO: 78, and the VL domain CDR1, CDR2 and CDR3 sequences of SEQ ID NO: 80. In some examples, the VH domain CDR1, CDR2 and CDR3 sequences respectively comprise residues 26-33, 51-58, and 96-120 of SEQ ID NO: 78, and the VL domain CDR1, CDR2 and CDR3 sequences respectively comprise residues 26-31, 49-51, and 87-98 of SEQ ID NO: 80. In specific examples, the amino acid sequence of the VH domain is at least 85%, at least 90%, at least 95%, at least 96%, at least 97%, at least 98% or at least 99% identical to SEQ ID NO: 78, and the amino acid sequence of the VL domain is at least 85%, at least 90%, at least 95%, at least 96%, at least 97%, at least 98% or at least 99% identical to SEQ ID NO: 80. In particular non-limiting examples, the amino acid sequence of the VH domain comprises SEQ ID NO: 78, and the amino acid sequence of the VL domain comprises SEQ ID NO: 80.

In some aspects, the monoclonal antibody includes the VH domain CDR1, CDR2 and CDR3 sequences of SEQ ID NO: 82, and the VL domain CDR1, CDR2 and CDR3 sequences of SEQ ID NO: 84. In some examples, the VH domain CDR1, CDR2 and CDR3 sequences respectively comprise residues 26-33, 51-58, and 96-112 of SEQ ID NO: 82, and the VL domain CDR1, CDR2 and CDR3 sequences respectively comprise residues 27-33, 51-53, and 89-99 of SEQ ID NO: 84. In specific examples, the amino acid sequence of the VH domain is at least 85%, at least 90%, at least 95%, at least 96%, at least 97%, at least 98% or at least 99% identical to SEQ ID NO: 82, and the amino acid sequence of the VL domain is at least 85%, at least 90%, at least 95%, at least 96%, at least 97%, at least 98% or at least 99% identical to SEQ ID NO: 84. In particular non-limiting examples, the amino acid sequence of the VH domain comprises SEQ ID NO: 82, and the amino acid sequence of the VL domain comprises SEQ ID NO: 84.

In some aspects, the monoclonal antibody includes the VH domain CDR1, CDR2 and CDR3 sequences of SEQ ID NO: 86, and the VL domain CDR1, CDR2 and CDR3 sequences of SEQ ID NO: 88. In some examples, the VH domain CDR1, CDR2 and CDR3 sequences respectively comprise residues 26-33, 51-58, and 96-112 of SEQ ID NO: 86, and the VL domain CDR1, CDR2 and CDR3 sequences respectively comprise residues 27-32, 50-52, and 88-98 of SEQ ID NO: 88. In specific examples, the amino acid sequence of the VH domain is at least 85%, at least 90%, at least 95%, at least 96%, at least 97%, at least 98% or at least 99% identical to SEQ ID NO: 86, and the amino acid sequence of the VL domain is at least 85%, at least 90%, at least 95%, at least 96%, at least 97%, at least 98% or at least 99% identical to SEQ ID NO: 88. In particular non-limiting examples, the amino acid sequence of the VH domain comprises SEQ ID NO: 86, and the amino acid sequence of the VL domain comprises SEQ ID NO: 88.

In some aspects, the monoclonal antibody includes the VH domain CDR1, CDR2 and CDR3 sequences of SEQ ID NO: 90, and the VL domain CDR1, CDR2 and CDR3 sequences of SEQ ID NO: 92. In some examples, the VH domain CDR1, CDR2 and CDR3 sequences respectively comprise residues 26-33, 51-58, and 96-121 of SEQ ID NO: 90, and the VL domain CDR1, CDR2 and CDR3 sequences respectively comprise residues 27-32, 50-52, and 88-99 of SEQ ID NO: 92. In specific examples, the amino acid sequence of the VH domain is at least 85%, at least 90%, at least 95%, at least 96%, at least 97%, at least 98% or at least 99% identical to SEQ ID NO: 90, and the amino acid sequence of the VL domain is at least 85%, at least 90%, at least 95%, at least 96%, at least 97%, at least 98% or at least 99% identical to SEQ ID NO: 92. In particular non-limiting examples, the amino acid sequence of the VH domain comprises SEQ ID NO: 90, and the amino acid sequence of the VL domain comprises SEQ ID NO: 92.

In some aspects, the monoclonal antibody includes the VH domain CDR1, CDR2 and CDR3 sequences of SEQ ID NO: 94, and the VL domain CDR1, CDR2 and CDR3 sequences of SEQ ID NO: 96. In some examples, the VH domain CDR1, CDR2 and CDR3 sequences respectively comprise residues 26-33, 51-57, and 95-109 of SEQ ID NO: 94, and the VL domain CDR1, CDR2 and CDR3 sequences respectively comprise residues 26-34, 52-54, and 90-101 of SEQ ID NO: 96. In specific examples, the amino acid sequence of the VH domain is at least 85%, at least 90%, at least 95%, at least 96%, at least 97%, at least 98% or at least 99% identical to SEQ ID NO: 94, and the amino acid sequence of the VL domain is at least 85%, at least 90%, at least 95%, at least 96%, at least 97%, at least 98% or at least 99% identical to SEQ ID NO: 96. In particular non-limiting examples, the amino acid sequence of the VH domain comprises SEQ ID NO: 94, and the amino acid sequence of the VL domain comprises SEQ ID NO: 96.

In some aspects, the monoclonal antibody includes the VH domain CDR1, CDR2 and CDR3 sequences of SEQ ID NO: 98, and the VL domain CDR1, CDR2 and CDR3 sequences of SEQ ID NO: 100. In some examples, the VH domain CDR1, CDR2 and CDR3 sequences respectively comprise residues 26-33, 51-58, and 96-121 of SEQ ID NO: 98, and the VL domain CDR1, CDR2 and CDR3 sequences respectively comprise residues 27-32, 50-52, and 88-99 of SEQ ID NO: 100. In specific examples, the amino acid sequence of the VH domain is at least 85%, at least 90%, at least 95%, at least 96%, at least 97%, at least 98% or at least 99% identical to SEQ ID NO: 98, and the amino acid sequence of the VL domain is at least 85%, at least 90%, at least 95%, at least 96%, at least 97%, at least 98% or at least 99% identical to SEQ ID NO: 100. In particular non-limiting examples, the amino acid sequence of the VH domain comprises SEQ ID NO: 98, and the amino acid sequence of the VL domain comprises SEQ ID NO: 100.

In some aspects, the monoclonal antibody includes the VH domain CDR1, CDR2 and CDR3 sequences of SEQ ID NO: 102, and the VL domain CDR1, CDR2 and CDR3 sequences of SEQ ID NO: 104. In some examples, the VH domain CDR1, CDR2 and CDR3 sequences respectively comprise residues 26-33, 51-58, and 97-117 of SEQ ID NO: 102, and the VL domain CDR1, CDR2 and CDR3 sequences respectively comprise residues 27-32, 50-52, and 89-98 of SEQ ID NO: 104. In specific examples, the amino acid sequence of the VH domain is at least 85%, at least 90%, at least 95%, at least 96%, at least 97%, at least 98% or at least 99% identical to SEQ ID NO: 102, and the amino acid sequence of the VL domain is at least 85%, at least 90%, at least 95%, at least 96%, at least 97%, at least 98% or at least 99% identical to SEQ ID NO: 104. In particular non-limiting examples, the amino acid sequence of the VH domain comprises SEQ ID NO: 102, and the amino acid sequence of the VL domain comprises SEQ ID NO: 104.

In some aspects, the monoclonal antibody includes the VH domain CDR1, CDR2 and CDR3 sequences of SEQ ID NO: 106, and the VL domain CDR1, CDR2 and CDR3 sequences of SEQ ID NO: 108. In some examples, the VH domain CDR1, CDR2 and CDR3 sequences respectively comprise residues 26-33, 51-58, and 97-112 of SEQ ID NO: 106, and the VL domain CDR1, CDR2 and CDR3 sequences respectively comprise residues 26-33, 51-53, and 92-101 of SEQ ID NO: 108. In specific examples, the amino acid sequence of the VH domain is at least 85%, at least 90%, at least 95%, at least 96%, at least 97%, at least 98% or at least 99% identical to SEQ ID NO: 106, and the amino acid sequence of the VL domain is at least 85%, at least 90%, at least 95%, at least 96%, at least 97%, at least 98% or at least 99% identical to SEQ ID NO: 108. In particular non-limiting examples, the amino acid sequence of the VH domain comprises SEQ ID NO: 106, and the amino acid sequence of the VL domain comprises SEQ ID NO: 108.

In some aspects, the monoclonal antibody includes the VH domain CDR1, CDR2 and CDR3 sequences of SEQ ID NO: 110, and the VL domain CDR1, CDR2 and CDR3 sequences of SEQ ID NO: 112. In some examples, the VH domain CDR1, CDR2 and CDR3 sequences respectively comprise residues 26-33, 51-58, and 97-112 of SEQ ID NO: 110, and the VL domain CDR1, CDR2 and CDR3 sequences respectively comprise residues 26-33, 51-53, and 90-100 of SEQ ID NO: 112. In specific examples, the amino acid sequence of the VH domain is at least 85%, at least 90%, at least 95%, at least 96%, at least 97%, at least 98% or at least 99% identical to SEQ ID NO: 110, and the amino acid sequence of the VL domain is at least 85%, at least 90%, at least 95%, at least 96%, at least 97%, at least 98% or at least 99% identical to SEQ ID NO: 112. In particular non-limiting examples, the amino acid sequence of the VH domain comprises SEQ ID NO: 110, and the amino acid sequence of the VL domain comprises SEQ ID NO: 112.

In some aspects, the monoclonal antibody includes the VH domain CDR1, CDR2 and CDR3 sequences of SEQ ID NO: 114, and the VL domain CDR1, CDR2 and CDR3 sequences of SEQ ID NO: 116. In some examples, the VH domain CDR1, CDR2 and CDR3 sequences respectively comprise residues 26-33, 51-58, and 97-110 of SEQ ID NO: 114, and the VL domain CDR1, CDR2 and CDR3 sequences respectively comprise residues 26-34, 52-54, and 91-101 of SEQ ID NO: 116. In specific examples, the amino acid sequence of the VH domain is at least 85%, at least 90%, at least 95%, at least 96%, at least 97%, at least 98% or at least 99% identical to SEQ ID NO: 114, and the amino acid sequence of the VL domain is at least 85%, at least 90%, at least 95%, at least 96%, at least 97%, at least 98% or at least 99% identical to SEQ ID NO: 116. In particular non-limiting examples, the amino acid sequence of the VH domain comprises SEQ ID NO: 114, and the amino acid sequence of the VL domain comprises SEQ ID NO: 116.

In some aspects, the monoclonal antibody includes the VH domain CDR1, CDR2 and CDR3 sequences of SEQ ID NO: 118, and the VL domain CDR1, CDR2 and CDR3 sequences of SEQ ID NO: 120. In some examples, the VH domain CDR1, CDR2 and CDR3 sequences respectively comprise residues 26-33, 51-58, and 97-107 of SEQ ID NO: 118, and the VL domain CDR1, CDR2 and CDR3 sequences respectively comprise residues 26-33, 51-53, and 90-100 of SEQ ID NO: 120. In specific examples, the amino acid sequence of the VH domain is at least 85%, at least 90%, at least 95%, at least 96%, at least 97%, at least 98% or at least 99% identical to SEQ ID NO: 118, and the amino acid sequence of the VL domain is at least 85%, at least 90%, at least 95%, at least 96%, at least 97%, at least 98% or at least 99% identical to SEQ ID NO: 120. In particular non-limiting examples, the amino acid sequence of the VH domain comprises SEQ ID NO: 118, and the amino acid sequence of the VL domain comprises SEQ ID NO: 120.

In some aspects, the monoclonal antibody includes the VH domain CDR1, CDR2 and CDR3 sequences of SEQ ID NO: 122, and the VL domain CDR1, CDR2 and CDR3 sequences of SEQ ID NO: 124. In some examples, the VH domain CDR1, CDR2 and CDR3 sequences respectively comprise residues 26-33, 51-58, and 97-111 of SEQ ID NO: 122, and the VL domain CDR1, CDR2 and CDR3 sequences respectively comprise residues 26-33, 51-53, and 90-100 of SEQ ID NO: 124. In specific examples, the amino acid sequence of the VH domain is at least 85%, at least 90%, at least 95%, at least 96%, at least 97%, at least 98% or at least 99% identical to SEQ ID NO: 122, and the amino acid sequence of the VL domain is at least 85%, at least 90%, at least 95%, at least 96%, at least 97%, at least 98% or at least 99% identical to SEQ ID NO: 124. In particular non-limiting examples, the amino acid sequence of the VH domain comprises SEQ ID NO: 122, and the amino acid sequence of the VL domain comprises SEQ ID NO: 124.

In some aspects, the monoclonal antibody includes the VH domain CDR1, CDR2 and CDR3 sequences of SEQ ID NO: 126, and the VL domain CDR1, CDR2 and CDR3 sequences of SEQ ID NO: 128. In some examples, the VH domain CDR1, CDR2 and CDR3 sequences respectively comprise residues 26-33, 51-58, and 97-111 of SEQ ID NO: 126, and the VL domain CDR1, CDR2 and CDR3 sequences respectively comprise residues 26-33, 51-53, and 92-100 of SEQ ID NO: 128. In specific examples, the amino acid sequence of the VH domain is at least 85%, at least 90%, at least 95%, at least 96%, at least 97%, at least 98% or at least 99% identical to SEQ ID NO: 126, and the amino acid sequence of the VL domain is at least 85%, at least 90%, at least 95%, at least 96%, at least 97%, at least 98% or at least 99% identical to SEQ ID NO: 128. In particular non-limiting examples, the amino acid sequence of the VH domain comprises SEQ ID NO: 126, and the amino acid sequence of the VL domain comprises SEQ ID NO: 128.

In some aspects, the monoclonal antibody includes the VH domain CDR1, CDR2 and CDR3 sequences of SEQ ID NO: 130, and the VL domain CDR1, CDR2 and CDR3 sequences of SEQ ID NO: 132. In some examples, the VH domain CDR1, CDR2 and CDR3 sequences respectively comprise residues 26-33, 51-57, and 96-110 of SEQ ID NO: 130, and the VL domain CDR1, CDR2 and CDR3 sequences respectively comprise residues 26-31, 49-51, and 88-98 of SEQ ID NO: 132. In specific examples, the amino acid sequence of the VH domain is at least 85%, at least 90%, at least 95%, at least 96%, at least 97%, at least 98% or at least 99% identical to SEQ ID NO: 130, and the amino acid sequence of the VL domain is at least 85%, at least 90%, at least 95%, at least 96%, at least 97%, at least 98% or at least 99% identical to SEQ ID NO: 132. In particular non-limiting examples, the amino acid sequence of the VH domain comprises SEQ ID NO: 130, and the amino acid sequence of the VL domain comprises SEQ ID NO: 132.

In some aspects, the monoclonal antibody includes the VH domain CDR1, CDR2 and CDR3 sequences of SEQ ID NO: 134, and the VL domain CDR1, CDR2 and CDR3 sequences of SEQ ID NO: 136. In some examples, the VH domain CDR1, CDR2 and CDR3 sequences respectively comprise residues 26-33, 51-58, and 97-111 of SEQ ID NO: 134, and the VL domain CDR1, CDR2 and CDR3 sequences respectively comprise residues 26-34, 52-54, and 91-101 of SEQ ID NO: 136. In specific examples, the amino acid sequence of the VH domain is at least 85%, at least 90%, at least 95%, at least 96%, at least 97%, at least 98% or at least 99% identical to SEQ ID NO: 134, and the amino acid sequence of the VL domain is at least 85%, at least 90%, at least 95%, at least 96%, at least 97%, at least 98% or at least 99% identical to SEQ ID NO: 136. In particular non-limiting examples, the amino acid sequence of the VH domain comprises SEQ ID NO: 134, and the amino acid sequence of the VL domain comprises SEQ ID NO: 136.

In some aspects, the monoclonal antibody is an IgG, IgM or IgA. In some examples, the IgG is IgG1. In other examples, the IgG is IgG2, IgG3 or IgG4.

In some aspects, the monoclonal antibody includes a heavy chain constant region and/or a light chain constant region, such as a human or NHP heavy chain constant region and/or light chain constant region.

In some aspects, the heavy chain constant region of the monoclonal antibody includes one or more amino acid substitutions to optimize in vivo half-life of the antibody. The serum half-life of IgG antibodies is regulated by the neonatal Fc receptor (FcRn). Thus, in particular aspects, the antibody includes an amino acid substitution that increases binding to the FcRn. Several such substitutions are known, such as substitutions at IgG constant regions T250Q and M428L (see, e.g., Hinton et al., J Immunol., 176:346-356, 2006); M428L and N434S (the “LS” mutation, see, e.g., Zalevsky, et al., Nature Biotechnology, 28:157-159, 2010); N434A (see, e.g., Petkova et al., Int. Immunol., 18:1759-1769, 2006); T307A, E380A, and N434A (see, e.g., Petkova et al., Int. Imnunol., 18:1759-1769, 2006); and M252Y, S254T, and T256E (see, e.g., Dall'Acqua et al., J. Biol. Chem., 281:23514-23524, 2006). In some examples, the monoclonal antibody includes a human IgG1 constant region having the M428L and N434S substitutions. The disclosed monoclonal antibodies can also be linked to a Fe polypeptide including any of the substitutions listed above, for example, the Fe polypeptide can include the M428L and N434S substitutions.

In some examples of the disclosed monoclonal antibodies, the human heavy chain constant region is an IgG1 constant region comprising the amino acid sequence of SEQ ID NO: 149. In some examples, the human light chain constant region is a lambda light chain constant region comprising the amino acid sequence of SEQ ID NO: 150, or a kappa light chain constant region comprising the amino acid sequence of SEQ ID NO: 151.

In some examples of the disclosed monoclonal antibodies, the amino acid sequence of the human heavy chain includes SEQ ID NO: 139, or residues 20-470 of SEQ ID NO: 139; and/or the amino acid sequence of the human light chain includes SEQ ID NO: 140, or residues 20-240 of SEQ ID NO: 140. In other examples, the amino acid sequence of the human heavy chain includes SEQ ID NO: 141, or residues 20-473 of SEQ ID NO: 141; and/or the amino acid sequence of the human light chain includes SEQ ID NO: 142, or residues 20-235 of SEQ ID NO: 142. In yet other examples, the amino acid sequence of the human heavy chain includes SEQ ID NO: 143, or residues 20-474 of SEQ ID NO: 143; and/or the amino acid sequence of the human light chain includes SEQ ID NO: 144, or residues 20-234 of SEQ ID NO: 144.

In some examples, the heavy chain constant region and the light chain constant region are macaque heavy chain and light chain constant regions. In particular examples, the macaque heavy chain constant region is an IgG1 constant region comprising the amino acid sequence of SEQ ID NO: 147; and/or the macaque light chain constant region is a lambda light chain constant region comprising the amino acid sequence of SEQ ID NO: 148. In specific non-limiting examples, the amino acid sequence of the macaque heavy chain includes SEQ ID NO: 137, or residues 20-474 of SEQ ID NO: 137; and/or the amino acid sequence of the macaque light chain includes SEQ ID NO: 138, or residues 20-236 of SEQ ID NO: 138.

In some aspects, the monoclonal antibody is an antigen-binding fragment, which includes a heavy chain variable region and a light chain variable region, and specifically binds SUDV and/or EBOV GP.

Non-limiting examples of antigen-binding fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{H} and V_{L} expressed as two chains; and
(5) Single chain antibody (such as scFv), defined as a genetically engineered molecule containing the variable region of the light chain, the variable region of the heavy chain, linked by a suitable polypeptide linker as a genetically fused single chain molecule. A scFv is a fusion protein in which a V_{L} of an immunoglobulin and a V_{H} of an immunoglobulin are bound by a linker (see, for example, Ahmad et al., Clin. Dev. Immunol., 2012: 980250, 2012; Mabry and Snavely, IDrugs, 13:543-549, 2010). The intramolecular orientation of the VH-domain and the V_{L}-domain in a scFv, is not decisive for the provided antibodies (for example, for the provided multispecific antibodies). Thus, scFvs with both possible arrangements (V_{H} domain-linker domain-V_{L} domain; V_{L} domain-linker domain-V_{H} domain) may be used.
(6) A dimer of a single chain antibody (scFv₂), defined as a dimer of a scFv. This has also been termed a “miniantibody.”

Methods of making these fragments are known (see for example, Harlow and Lane, Antibodies: A Laboratory Manual, 2^{nd}, Cold Spring Harbor Laboratory, New York, 2013).

In some aspects, the antigen binding fragment is an Fv antibody, which is typically about 25 kDa and contains a complete antigen-binding site with three CDRs per each heavy chain and each light chain. To produce Fv antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the V_{H} and the V_{L}, are chemically linked by disulfide bonds.

In an additional example, the Fv fragments comprise V_{H} and V_{L} chains connected by a peptide linker. These single-chain antigen binding proteins (scFv) are prepared by constructing a nucleic acid molecule encoding the V_{H} and V_{L} domains connected by an oligonucleotide. The nucleic acid molecule is inserted into an expression vector, which is subsequently introduced into a host cell such as a mammalian cell. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; Ahmad et al., Clin. Dev. Immunol., 2012: 980250, 2012; Mabry and Snavely, IDrugs, 13:543-549, 2010). Dimers of a single chain antibody (scFV₂) are also contemplated.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

In some examples, one or more of the heavy and/or light chain CDRs from a disclosed antibody is expressed on the surface of another protein, such as a scaffold protein. The expression of domains of antibodies on the surface of a scaffolding protein are known (see, for example, Liu et al., J. Virology 85(17): 8467-8476, 2011). Such expression creates a chimeric protein that retains the binding for GP. In some specific examples, one or more of the heavy chain CDRs is grafted onto a scaffold protein, such as one or more of heavy chain CDR1, CDR2, and/or CDR3. One or more CDRs can also be included in a diabody or another type of single chain antibody molecule.

In some aspects, the monoclonal antibody is a fully human antibody or a humanized antibody. In other aspects, the monoclonal antibody is a NHP antibody, such as a macaque antibody.

In some aspects, the monoclonal antibody is linked to an effector molecule (such as a toxin or drug) or a detectable label. In some examples, the detectable label is a fluorescent, enzymatic, radioactive or nucleic acid label. In some instances, the antibody can be conjugated to a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody can also be conjugated with enzymes that are useful for detection, such as horseradish peroxidase, β-galactosidase, luciferase, alkaline phosphatase, glucose oxidase and the like. When an antibody is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody may also be conjugated with biotin, and detected through indirect measurement of avidin or streptavidin binding. It should be noted that the avidin itself can be conjugated with an enzyme or a fluorescent label.

The antibody can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody may also be labeled with a predetermined polypeptide epitopes recognized by a secondary reporter (such as leucine zipper pair sequences, binding sites for secondary antibodies, metal binding domains, epitope tags).

The antibody can also be conjugated with a radiolabeled amino acid. The radiolabel may be used for both diagnostic and therapeutic purposes. For instance, the radiolabel may be used to detect GP and GP-expressing cells by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

Means of detecting such detectable markers are well known. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of effector molecule or detectable marker moieties per antibody in a conjugate can range, for example, from 1 to 20 moieties per antibody. In certain aspects, the average number of effector molecules or detectable marker moieties per antibody in a conjugate range from about 1 to about 2, from about 1 to about 3, about 1 to about 8; from about 2 to about 6; from about 3 to about 5; or from about 3 to about 4. The loading (for example, effector molecule/antibody ratio) of an conjugate may be controlled in different ways, for example, by: (i) limiting the molar excess of effector molecule-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number or position of linker-effector molecule attachments.

Further provided herein are monoclonal antibodies that bind to the same epitope as a monoclonal antibody disclosed herein, such as a monoclonal antibody that neutralizes SUDV and/or EBOV. Antibodies that bind to such an epitope can be identified based on their ability to cross-compete (for example, to competitively inhibit the binding of, in a statistically significant manner) with the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L and 396L antibodies provided herein in SUDV/EBOV GP binding assays (such as those described in the Examples). An antibody “competes” for binding when the competing antibody inhibits SUDV/EBOV GP binding of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L antibody by more than 50%, in the presence of competing antibody concentrations higher than 10⁶×K_{D} of the competing antibody. In a certain aspect, the antibody that binds to the same epitope on SUDV or EBOV GP as the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L antibody is a human monoclonal antibody. Human antibodies that bind to the same epitope on GP to which the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L antibody binds can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008). Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For a review of methods for obtaining human antibodies from transgenic animals, see Lonberg, Nat. Biotech. 23:1117-1125 (2005) (see also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, for example, by combining with a different human constant region.

Human antibodies that bind to the same epitope on EBOV GP to which the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L antibody binds can also be made by hybridoma-based methods. Human myeloma and mouse-human heteromyeloma cell lines for the production of human monoclonal antibodies have been described (see, for example, Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies that specifically bind to the same epitope on EBOV GP as 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L can also be isolated by screening combinatorial libraries for antibodies with the desired binding characteristics. For example, a variety of methods are known for generating phage display libraries and screening such libraries for antibodies possessing the desired binding characteristics. Such methods are reviewed, for example, in Hoogenboom et al. in Methods in Molecular Biology 178:1-37 (O'Brien et al., ed., Human Press, Totowa, N.J., 2001) and further described, for example, in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (for example, from humans) to provide a single source of antibodies to a wide range of non-self and also self-antigens without any immunization as described by Griffiths et al., EMBO J, 12: 725-734 (1993). Finally, naïve libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

In certain aspects, amino acid sequence variants of the monoclonal antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In certain aspects, antibody variants having one or more amino acid substitutions are provided. Sites of interest for substitutional mutagenesis include the CDRs and the framework regions. Amino acid substitutions may be introduced into an antibody of interest and the products screened for a desired activity, for example, retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield.

In some aspects, the heavy chain of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence of an antibody heavy chain (or VH domain) disclosed herein. In some aspects, the light chain of the antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence of an antibody light chain (or VL domain) disclosed herein.

In some aspects, the antibody can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody, or the light chain of the antibody, or the heavy and light chains of the antibody, compared to a known framework region, or compared to the framework regions of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L antibody, and maintain the specific binding activity for SUDV and/or EBOV GP.

### C. Bispecific Monoclonal Antibodies

Also provided herein are bispecific monoclonal antibodies that include a GP-specific monoclonal antibody disclosed herein. The bispecific monoclonal antibodies include a first antigen binding portion and a second antigen binding portion, wherein at least one of the first antigen binding portion and the second antigen binding portion includes the CDR sequences (or the complete variable domains) of any one of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L and 396L antibodies. In some aspects, the first antigen binding portion and the second antigen binding portion each individually include the CDR sequences (or the complete variable domains) of two different antibodies disclosed herein. In other aspects, the first antigen binding portion includes the CDR sequences (or complete variable domains) of any one of the antibodies disclosed herein, and the second antigen binding portion includes the CDR sequences (or complete variable domains) of a different Ebolavirus-specific monoclonal antibody.

In some aspects, the bispecific monoclonal antibodies are generated using CrossMab technology (see, e.g., US 2017/0129962). In the CrossMab format, each arm of the bispecific antibody contains a different antibody variable fragment (Fv) domain. To maintain the specificity of light chain and heavy chain association in the Fv domains, the CrossMab format uses two features. First, one Fc domain of the bispecific antibody contains so-called “knob” residues and the other Fc domain contains “hole” residues (see FIG. 26). The “knob” and “hole” residues prefer to associate with each other over self-association. This promotes hetero-associations between heavy chains. Second, to promote proper light chain associations, one of the arms of the antibody has the heavy chain CH1 domain and the light chain CL domain swapped and is referred to as the “swapped” arm (FIG. 26). In some examples herein, the Fe domains include one or more modifications, such as modifications that extend half-life of the bispecific antibody. In specific examples, the modifications include the “LS” substitutions.

In some aspects of the bispecific monoclonal antibody, the first antigen binding portion includes the CDR sequences (or complete variable domains) of antibody 545S and the second antigen binding portion includes the CDR sequences (or the complete variable domains) of antibody 523S.

In some examples, the first antigen binding portion includes a VH domain and a VL domain, wherein the VH domain includes the CDR1, CDR2 and CDR3 sequences of SEQ ID NO: 14 and the VL domain includes the CDR1, CDR2 and CDR3 sequences of SEQ ID NO: 16; and the second antigen binding portion includes a VH domain and a VL domain, wherein the VH domain includes the CDR1, CDR2 and CDR3 sequences of SEQ ID NO: 18 and the VL domain includes the CDR1, CDR2 and CDR3 sequences of SEQ ID NO: 20. In particular examples, the VH domain of the first antigen binding portion includes the amino acid sequence of SEQ ID NO: 14 and the VL domain of the first antigen binding portion includes the amino acid sequence of SEQ ID NO: 16; and the VH domain of the second antigen binding portion includes the amino acid sequence of SEQ ID NO: 18 and the VL domain of the second antigen binding portion includes the amino acid sequence of SEQ ID NO: 20.

In one non-limiting example, the first antigen binding portion includes a heavy chain comprising SEQ ID NO: 152 or residues 20-473 of SEQ ID NO: 152, and a light chain comprising SEQ ID NO: 153 or residues 20-235 of SEQ ID NO: 153; and/or the second antigen binding portion includes a heavy chain comprising SEQ ID NO: 154 or residues 20-472 of SEQ ID NO: 154, and a light chain comprising SEQ ID NO: 155 or residues 20-239 of SEQ ID NO: 155.

In another non-limiting example, the first antigen binding portion includes a heavy chain comprising SEQ ID NO: 156 or residues 20-470 of SEQ ID NO: 156, and a light chain comprising SEQ ID NO: 157 or residues 20-240 of SEQ ID NO: 157; and/or the second antigen binding portion includes a heavy chain comprising SEQ ID NO: 158 or residues 20-474 of SEQ ID NO: 158, and a light chain comprising SEQ ID NO: 159 or residues 20-234 of SEQ ID NO: 159.

Also provided herein are other types of multi-specific antibodies, such as trispecific antibodies. Such multispecific antibodies can be produced by known methods, such as crosslinking two or more antibodies, or antigen binding fragments (such as scFvs) of the same type or of different types. Exemplary methods of making multispecific antibodies include those described in PCT Pub. No. WO 2013/163427. Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are available from Pierce Chemical Company, Rockford, Ill.

Various types of multi-specific antibodies are known. Bispecific single chain antibodies can be encoded by a single nucleic acid molecule. Examples of bispecific single chain antibodies, as well as methods of constructing such antibodies are known in the art (see, e.g., U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538, incorporated by reference herein). Additional examples of bispecific single chain antibodies can be found in PCT application No. WO 99/54440; Mack, J. Immunol., 158:3965-3970, 1997; Mack, PNAS, 92:7021-7025, 1995; Kufer, Cancer Immunol. Immunother., 45:193-197, 1997; Loffler, Blood, 95:2098-2103, 2000; and Bruhl, J. Immunol., 166:2420-2426, 2001. Production of bispecific Fab-scFv (“bibody”) molecules are described, for example, in Schoonjans et al. (J. Immunol. 165:7050-57, 2000) and Willems et al. (J Chromatogr B Analyt Technol Biomed Life Sci. 786:161-76, 2003). For bibodies, a scFv molecule can be fused to one of the VL-CL (L) or VH-CH1 chains, e.g., to produce a bibody one scFv is fused to the C-terminus of a Fab chain.

### D. Nucleic Acid Molecules, Vectors and Host Cells

Isolated nucleic acid molecules and vectors encoding the monoclonal antibodies and bispecific monoclonal antibodies disclosed herein are also provided. Further provided are isolated cells that include a nucleic acid molecule or vector disclosed herein.

Nucleic acids molecules (for example, DNA, cDNA and RNA molecules) encoding the amino acid sequences of the disclosed monoclonal antibodies and bispecific antibodies that specifically bind SUDV and/or EBOV GP can readily be produced by one of skill in the art, using the amino acid sequences provided herein (such as the CDR sequences and V_{H} and V_{L} sequences), sequences available in the art (such as framework or constant region sequences), and the genetic code. In several aspects, a nucleic acid molecule can encode the V_{H}, the V_{L}, or both the V_{H} and V_{L} (for example in a bicistronic expression vector) of a disclosed antibody. In several aspects, the nucleic acid molecules can be expressed in a host cell (such as a mammalian cell) to produce a disclosed antibody or antigen binding fragment.

In some aspects, the isolated nucleic acid molecule encodes a VH domain, a VL domain, or both a VH domain and a VL domain, of a monoclonal antibody disclosed herein. In some examples, the nucleic acid molecule encoding the VH domain and/or the VL domain of the monoclonal antibody includes the nucleic acid sequences set forth as SEQ ID NOs: 1 and/or 3, respectively, or degenerate variants thereof; SEQ ID NOs: 5 and/or 7, respectively, or degenerate variants thereof; SEQ ID NOs: 9 and/or 11, respectively, or degenerate variants thereof; SEQ ID NOs: 13 and/or 15, respectively, or degenerate variants thereof; SEQ ID NOs: 17 and/or 19, respectively, or degenerate variants thereof; SEQ ID NOs: 21 and/or 23, respectively, or degenerate variants thereof; SEQ ID NOs: 25 and/or 27, respectively, or degenerate variants thereof; SEQ ID NOs: 29 and/or 31, respectively, or degenerate variants thereof; SEQ ID NOs: 33 and/or 35, respectively, or degenerate variants thereof; SEQ ID NOs: 37 and/or 39, respectively, or degenerate variants thereof; SEQ ID NOs: 41 and/or 43, respectively, or degenerate variants thereof; SEQ ID NOs: 45 and/or 47, respectively, or degenerate variants thereof; SEQ ID NOs: 49 and/or 51, respectively, or degenerate variants thereof; SEQ ID NOs: 53 and/or 55, respectively, or degenerate variants thereof; SEQ ID NOs: 57 and/or 59, respectively, or degenerate variants thereof; SEQ ID NOs: 61 and/or 63, respectively, or degenerate variants thereof; SEQ ID NOs: 65 and/or 67, respectively, or degenerate variants thereof; SEQ ID NOs: 69 and/or 71, respectively, or degenerate variants thereof; SEQ ID NOs: 73 and/or 75, respectively, or degenerate variants thereof; SEQ ID NOs: 77 and/or 79, respectively, or degenerate variants thereof; SEQ ID NOs: 81 and/or 83, respectively, or degenerate variants thereof; SEQ ID NOs: 85 and/or 87, respectively, or degenerate variants thereof; SEQ ID NOs: 89 and/or 91, respectively, or degenerate variants thereof; SEQ ID NOs: 93 and/or 95, respectively, or degenerate variants thereof; SEQ ID NOs: 97 and/or 99, respectively, or degenerate variants thereof; SEQ ID NOs: 101 and/or 103, respectively, or degenerate variants thereof; SEQ ID NOs: 105 and/or 107, respectively, or degenerate variants thereof; SEQ ID NOs: 109 and/or 111, respectively, or degenerate variants thereof; SEQ ID NOs: 113 and/or 115, respectively, or degenerate variants thereof; SEQ ID NOs: 117 and/or 119, respectively, or degenerate variants thereof; SEQ ID NOs: 121 and/or 123, respectively, or degenerate variants thereof; SEQ ID NOs: 125 and/or 127, respectively, or degenerate variants thereof; SEQ ID NOs: 129 and/or 131, respectively, or degenerate variants thereof; or SEQ ID NOs: 133 and/or 135, respectively, or degenerate variants thereof.

In some aspects, the nucleic acid molecule encodes a bispecific monoclonal antibody disclosed herein, or a heavy chain or a light chain thereof. In some examples, the nucleic acid molecule encodes a heavy chain comprising SEQ ID NO: 152 or residues 20-473 of SEQ ID NO: 152; a heavy chain comprising SEQ ID NO: 154 or residues 20-472 of SEQ ID NO: 154; a heavy chain comprising SEQ ID NO: 156 or residues 20-470 of SEQ ID NO: 156; or a heavy chain comprising SEQ ID NO: 158 or residues 20-474 of SEQ ID NO: 158. In some examples, the nucleic acid molecule encodes a light chain comprising SEQ ID NO: 153 or residues 20-235 of SEQ ID NO: 153; a light chain comprising SEQ ID NO: 155 or residues 20-239 of SEQ ID NO: 155; a light chain comprising SEQ ID NO: 157 or residues 20-240 of SEQ ID NO: 157; or a light chain comprising SEQ ID NO: 159 or residues 20-234 of SEQ ID NO: 159.

In some aspects, the nucleic acid molecule is operably linked to a promoter.

Further provided herein are vectors that include a nucleic acid molecule disclosed herein, and isolated host cells that include a disclosed nucleic acid molecule or vector are also provided. In some examples, the host cell is a bacterial cell, yeast cell, insect cell or mammalian cell (such as a human cell or NHP cell).

One of skill in the art can readily use the genetic code to construct a variety of functionally equivalent nucleic acids, such as nucleic acids which differ in sequence but which encode the same antibody sequence. Nucleic acid sequences encoding antibodies, bispecific antibodies and conjugates that specifically bind SUDV and/or EBOV GP can be prepared by any suitable method including, for example, cloning of appropriate sequences, direct chemical synthesis, and nucleic acid amplification methods.

### E. Methods and Compositions

Also provided are methods of treating or inhibiting an Ebolavirus infection (such as a SUDV or EBOV infection) in a subject by administering to the subject a therapeutically effective amount of a monoclonal antibody, bispecific monoclonal antibody, nucleic acid molecule, vector, or composition disclosed herein. The methods can include post-infection treatment, post-exposure prophylaxis or pre-exposure prophylaxis. In some aspects, the method includes administering multiple (e.g., at least two, at least three, at least four, or at least five) different monoclonal antibodies and/or bispecific antibodies disclosed herein to the subject.

### 1. Methods of Inhibiting, Treating, and Preventing SUDV/EBOV Infection and Disease

Also provided are methods of treating or inhibiting an Ebolavirus infection (such as a SUDV or EBOV infection) in a subject by administering to the subject a therapeutically effective amount of a monoclonal antibody, bispecific monoclonal antibody, nucleic acid molecule, vector, or composition disclosed herein. The methods can include post-infection treatment, post-exposure prophylaxis or pre-exposure prophylaxis. In some aspects, the method includes administering multiple (e.g., at least two, at least three, at least four, or at least 5) different monoclonal antibodies and/or bispecific antibodies disclosed herein to the subject. In some examples, the method further includes administering to the subject one or more additional therapies for treating the Ebolavirus infection.

In some aspects, the monoclonal antibody, bispecific monoclonal antibody, nucleic acid molecule, vector or composition is administered no more than 2, no more than 3, no more than 4, no more than 5, no more than 6, no more than 7, no more than 8, no more than 9, no more than 10, no more than 15 or no more than 20 days following the Ebolavirus infection. In some examples, the subject has been exposed to an Ebolavirus but has not been diagnosed as having an Ebolavirus infection. In specific non-limiting examples, the monoclonal antibody, bispecific monoclonal antibody, nucleic acid molecule, vector or composition is administered no more than 2, no more than 3, no more than 4, no more than 5, no more than 6, no more than 7, no more than 8, no more than 9, no more than 10, no more than 15 or no more than 20 days following exposure to the Ebolavirus.

In other aspects, the subject has not yet been exposed to an Ebolavirus. In some examples, the monoclonal antibody, bispecific monoclonal antibody, nucleic acid molecule, vector or composition is administered about 16 weeks, about 14 weeks, about 12 weeks, about 10 weeks, about 8 weeks, about 6 weeks, about 4 weeks, about 2 weeks, about 1 one week, about 6 days, about 5 days, about 4 days, about 3 days, about 2 days and/or about 1 day prior to exposure to the Ebolavirus.

In some aspects, the monoclonal antibody, bispecific monoclonal antibody, nucleic acid molecule, vector or composition is administered in multiple doses, such as (but not limited to) two, three, four or five doses. In other aspects, the monoclonal antibody, bispecific monoclonal antibody, nucleic acid molecule, vector or composition is administered in a single dose.

Methods are disclosed herein for the inhibition (such as prevention) or treatment of an EBOV or SUDV infection or EVD, in a subject. Prevention can include inhibition of infection with SUDV or EBOV. The method can include administering to a subject a therapeutically effective amount of a disclosed monoclonal antibody, bispecific antibody, or conjugate (for example, an antibody conjugated to a toxin or drug) that specifically binds SUDV or EBOV GP, or a nucleic acid encoding such an antibody, bispecific antibody, or conjugate. In some examples, the monoclonal antibody, bispecific antibody, conjugate, or nucleic acid molecule can be used pre-exposure (for example, to prevent or inhibit SUDV or EBOV infection). In some examples, the antibody, bispecific antibody, conjugate, or nucleic acid molecule, can be used in post-exposure prophylaxis. In some examples, the antibody, bispecific antibody, conjugate, or nucleic acid molecule, can be used to eliminate or reduce the viral load of SUDV or EBOV in a subject infected with SUDV or EBOV. For example, a therapeutically effective amount of an antibody, bispecific antibody, conjugate, or nucleic acid molecule, can be administered to a subject with a SUDV or EBOV infection. In some examples, the antibody, bispecific antibody, conjugate, or nucleic acid molecule is modified such that it is directly cytotoxic to infected cells (for example, by conjugation to a toxin), or uses natural defenses such as complement, antibody dependent cellular cytotoxicity (ADCC), or phagocytosis by macrophages, or can be modified to increase the natural defenses.

The EVD or SUDV/EBOV infection in the subject does not need to be completely eliminated for the method to be effective. For example, the method can reduce or ameliorate EVD or SUDV/EBOV infection by a desired amount, for example by at least 10%, at least 20%, at least 50%, at least 60%, at least 70%, at least 80%, at least 90%, at least 95%, at least 98%, or even at least 100% (elimination of detectable SUDV or EBOV infection or EVD), as compared to SUDV or EBOV infection or EVD in the absence of the treatment.

In one non-limiting example, the method reduces viral titer in a subject with a SUDV or EBOV infection. For example, administration of a therapeutically effective amount of a disclosed SUDV or EBOV GP-specific antibody or bispecific antibody can reduce viral titer by at least 10%, at least 20%, at least 50%, at least 60%, at least 70%, at least 80%, at least 90%, at least 95%, at least 98%, or even at least 100% (elimination of detectable SUDV or EBOV) in the subject. Methods of determining the SUDV or EBOV viral titer in the subject are known, and include, for example, obtaining a blood sample from the subject and assaying the sample for SUDV or EBOV activity.

In several aspects, administration of a therapeutically effective amount of a disclosed antibody, bispecific antibody, conjugate, or nucleic acid molecule, results in a reduction in the establishment of SUDV or EBOV infection and/or reducing subsequent EVD progression in a subject. A reduction in the establishment of SUDV or EBOV infection and/or a reduction in subsequent EVD progression encompass any statistically significant reduction in SUDV or EBOV activity.

In several aspects, the subject can be selected for treatment, for example, a subject at risk of SUDV or EBOV infection, or known to have a SUDV or EBOV infection. In some aspects, a subject can be selected that is at risk of or known to have an infection with any virus of the Ebolavirus genus, such as BDBV, RESTV, SUDV, TAFV, BOMV or EBOV.

In several aspects, a method of preventing or inhibiting SUDV or EBOV infection of a cell is provided. The method includes contacting the cell with an effective amount of an antibody as disclosed herein. For example, the cell can be incubated with the effective amount of the antibody prior to or contemporaneous with incubation with the SUDV or EBOV. SUDV or EBOV infection of the cell does not need to be completely eliminated for the method to be effective. For example, a method can reduce SUDV or EBOV infection by a desired amount, for example by at least 10%, at least 20%, at least 50%, at least 60%, at least 70%, at least 80%, at least 90%, at least 95%, at least 98%, or even at least 100% (elimination of detectable SUDV or EBOV infected cells), as compared to SUDV or EBOV infection in the absence of the treatment. In some aspects, the cell is also contacted with an effective amount of an additional agent, such as anti-viral agent. The cell can be in vivo or in vitro.

Studies have shown that cocktails of EBOV neutralizing antibodies that target different epitopes of EBOV GP can treat macaques infected with ZEBOV (Qiu et al., Sci. Transl. Med., 4, 138ra81, 2012). Accordingly, in some examples, a subject is further administered one or more additional antibodies that bind SUDV or EBOV GP and that can neutralize SUDV or EBOV infection. For example, the subject can be administered a therapeutically effective amount of a set of antibodies including two or more of the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L and 396L antibodies disclosed herein. The antibodies can be administered as a cocktail (that is, as a single composition including the two or more antibodies), or can be administered sequentially.

In some examples, a subject is administered the DNA encoding the antibody to provide in vivo antibody production, for example using the cellular machinery of the subject. Immunization by nucleic acid constructs is well known in the art and taught, for example, in U.S. Pat. Nos. 5,643,578, and 5,593,972 and 5,817,637. U.S. Pat. No. 5,880,103 describes several methods of delivery of nucleic acids encoding to an organism. One approach to administration of nucleic acids is direct administration with plasmid DNA, such as with a mammalian expression plasmid. The nucleotide sequence encoding the disclosed antibody can be placed under the control of a promoter to increase expression. The methods include liposomal delivery of the nucleic acids. Such methods can be applied to the production of an antibody. In some aspects, a disclosed antibody is expressed in a subject using the pVRC8400 vector (described in Barouch et al., J. Virol, 79:8828-8834, 2005).

The nucleic acid molecules encoding the disclosed antibodies or bispecific antibodies can be included in a viral vector (or multiple vectors, particularly for bispecific antibodies), for example for expression of the antibody or bispecific antibody in a host cell, or a subject (such as a subject with or at risk of SUDV or EBOV infection). A number of viral vectors have been constructed that can be used to express the disclosed antibodies or bispecific antibodies, such as a retroviral vector, an adenoviral vector, or an adeno-associated virus (AAV) vector. In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

In one aspect, a nucleic acid encoding a disclosed antibody is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 g/kg to about 50 mg/kg, and typically are about 0.005 mg/kg to about 5 mg/kg (see, e.g., U.S. Pat. No. 5,589,466).

### 2. Dosages

A therapeutically effective amount of a SUDV or EBOV GP-specific antibody, bispecific antibody, conjugate, or nucleic acid molecule encoding such molecules, will depend upon the severity of the disease and/or infection and the general state of the patient's health. A therapeutically effective amount is that which provides either subjective relief of a symptom(s) or an objectively identifiable improvement as noted by the clinician or other qualified observer. The SUDV or EBOV GP-specific antibody, bispecific antibody, conjugate, or nucleic acid molecule encoding such molecules, can be administered in conjunction with another therapeutic agent, either simultaneously or sequentially.

Single or multiple administrations of a composition including a disclosed SUDV or EBOV GP-specific antibody, bispecific antibody, conjugate, or nucleic acid molecule encoding such molecules, can be administered depending on the dosage and frequency as required and tolerated by the patient. Compositions including the SUDV or EBOV GP-specific antibody, bispecific antibody, conjugate, or nucleic acid molecule encoding such molecules, should provide a sufficient quantity of at least one of the SUDV or EBOV GP-specific antibodies, bispecific antibodies, conjugates, or nucleic acid molecules to effectively treat the patient. The dosage can be administered once, but may be applied periodically until either a therapeutic result is achieved or until side effects warrant discontinuation of therapy. In one example, a dose of the antibody or antigen binding fragment is infused for thirty minutes every other day. In this example, about one to about ten doses can be administered, such as three or six doses can be administered every other day. In a further example, a continuous infusion is administered for about five to about ten days. The subject can be treated at regular intervals, such as daily, weekly, or monthly, until a desired therapeutic result is achieved. Generally, the dose is sufficient to treat or ameliorate symptoms or signs of disease without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The therapeutically effective dose can be determined from cell culture assays and animal studies.

In certain aspects, the antibody or antigen binding fragment that specifically binds SUDV or EBOV GP, or a nucleic acid molecule or vector encoding such a molecule, can be administered at a dose in the range of from about 1 to about 100 mg/kg, such as about 5-50 mg/kg, about 25-75 mg/kg, or about 40-60 mg/kg. In some aspects, the dosage can be administered at about 1, 2, 3, 4, 5, 6, 7, 8, 9, 10, 20, 30, 40, 50, 60, 70, 80, 90, 100, 150, 200, or 300 mg/kg, or other dose deemed appropriate by the treating physician. Further, the doses described herein can be administered according to the dosing frequency or frequency of administration described herein, including without limitation daily, every other day, 2 or 3 times per week, weekly, every 2 weeks, every 3 weeks, monthly, etc. In some aspects, the dosage is administered daily beginning at the time of diagnosis with SUDV or EBOV and until SUDV or EBOV symptoms are alleviated. Additional treatments, including additional courses of therapy with a disclosed agent can be performed as needed.

### 3. Modes of Administration

The SUDV or EBOV GP-specific antibody, bispecific antibody, conjugate, nucleic acid molecule, or composition, as well as additional agents, can be administered to subjects in various ways, including local and systemic administration, such as, for example, by injection subcutaneously, intravenously, intra-arterially, intraperitoneally, intramuscularly, intradermally, or intrathecally. In an aspect, a therapeutic agent is administered by a single subcutaneous, intravenous, intra-arterial, intraperitoneal, intramuscular, intradermal or intrathecal injection once a day. The therapeutic agent can also be administered by direct injection at or near the site of disease.

The therapeutic agent may also be administered orally in the form of microspheres, microcapsules, liposomes (uncharged or charged (such as cationic)), polymeric microparticles (such as polyamides, polylactide, polyglycolide, poly(lactide-glycolide)), microemulsions, and the like.

A further method of administration is by osmotic pump (for example, an Alzet pump) or mini-pump (for example, an Alzet mini-osmotic pump), which allows for controlled, continuous and/or slow-release delivery of the therapeutic agent or pharmaceutical composition over a pre-determined period. The osmotic pump or mini-pump can be implanted subcutaneously, or near a target site.

It will be apparent to one skilled in the art that the therapeutic agent or compositions thereof can also be administered by other modes. The therapeutic agent can be administered as pharmaceutical formulations suitable for, for example, oral (including buccal and sub-lingual), rectal, nasal, topical, pulmonary, vaginal or parenteral (including intramuscular, intraarterial, intrathecal, subcutaneous and intravenous) administration, or in a form suitable for administration by inhalation or insufflation. Depending on the intended mode of administration, the pharmaceutical formulations can be in the form of solid, semi-solid or liquid dosage forms, such as tablets, suppositories, pills, capsules, powders, liquids, suspensions, emulsions, creams, ointments, lotions, and the like. The formulations can be provided in unit dosage form suitable for single administration of a precise dosage. The formulations comprise an effective amount of a therapeutic agent, and one or more pharmaceutically acceptable excipients, carriers and/or diluents, and optionally one or more other biologically active agents.

### 4. Compositions

Compositions are provided that include one or more of the disclosed SUDV or EBOV GP-specific monoclonal antibodies, bispecific antibodies, conjugates, nucleic acid molecules or vectors, in a carrier. The compositions are useful, for example, for the treatment or detection of a SUDV or EBOV infection. The compositions can be prepared in unit dosage forms for administration to a subject. The amount and timing of administration are at the discretion of the treating physician to achieve the desired purposes. The SUDV or EBOV GP-specific antibody, bispecific antibody, conjugate, or nucleic acid molecule encoding such molecules can be formulated for systemic or local administration. In one example, the SUDV or EBOV GP-specific antibody, antigen binding fragment, conjugate, or nucleic acid molecule encoding such molecules, is formulated for parenteral administration, such as intravenous administration.

In some aspects, the compositions comprise a monoclonal antibody, bispecific antibody, or conjugate thereof, in at least 70%, at least 75%, at least 80%, at least 85%, at least 90%, at least 95%, at least 96%, at least 97%, at least 98% or at least 99% purity. In certain aspects, the compositions contain less than 10%, less than 5%, less than 4%, less than 3%, less than 2%, less than 1% or less than 0.5% of macromolecular contaminants, such as other mammalian (for example, human) proteins.

The compositions for administration can include a solution of the SUDV or EBOV GP-specific antibody, bispecific antibody, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration includes about 0.01 to about 30 mg/kg of antibody or antigen binding fragment or conjugate per subject per day (or the corresponding dose of a conjugate including the antibody or antigen binding fragment). Actual methods for preparing administrable compositions will be known or apparent to those skilled in the art and are described in more detail in such publications as Remington's Pharmaceutical Science, 22th ed., Pharmaceutical Press, London, UK (2012). In some aspects, the composition can be a liquid formulation including one or more antibodies or bispecific antibodies, in a concentration range from about 0.1 mg/kg to about 20 mg/kg, or from about 0.5 mg/kg to about 20 mg/kg, or from about 1 mg/kg to about 20 mg/kg, or from about 0.1 mg/kg to about 10 mg/kg, or from about 0.5 mg/kg to about 10 mg/kg, or from about 1 mg/kg to about 10 mg/kg.

The disclosed monoclonal antibodies, bispecific antibodies, conjugates, and nucleic acid encoding such molecules, can be provided in lyophilized form and rehydrated with sterile water before administration, although they are also provided in sterile solutions of known concentration. The antibody solution can then be added to an infusion bag containing 0.9% sodium chloride, USP, and administered according to standard protocols. Considerable experience is available in the art in the administration of antibody drugs, which have been marketed in the U.S. since the approval of RITUXAN® in 1997. Antibodies, bispecific antibodies, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 m so that only nanoparticles are administered intravenously. Microparticles are typically around 100 μm in diameter and are administered subcutaneously or intramuscularly (see, for example, Kreuter, Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the antibody compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### 5. Methods of Detection and Diagnosis

Further provided are methods of detecting SUDV or EBOV GP in a sample by contacting the sample with a disclosed monoclonal antibody or bispecific monoclonal antibody under conditions sufficient to form an immune complex, and detecting the presence of the immune complex in the sample. Similarly, provided are methods of diagnosing an Ebolavirus infection (such as a SUDV or EBOV infection) in a subject by contacting a biological sample from the subject with a disclosed monoclonal antibody or bispecific monoclonal antibody under conditions sufficient to form an immune complex, and detecting the presence of the immune complex in the sample.

The disclosed methods can be used for the detection of SUDV or EBOV GP in vitro or in vivo. In one example, expression of SUDV or EBOV GP is detected in a biological sample, and can be used to detect a SUDV or EBOV infection based on the presence of SUDV or EBOV GP in a sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, or administering to a subject, an antibody that specifically binds to SUDV or EBOV GP, or conjugate thereof (such as a conjugate including a detectable marker) under conditions sufficient to form an immune complex, and detecting the immune complex (for example, by detecting a detectable marker conjugated to the antibody.

In some aspects, the disclosed antibodies are used to test vaccines. For example, to test if a vaccine composition including SUDV or EBOV GP assumes a conformation including the SUDV or EBOV GP epitope to which the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L antibody binds. Thus, provided herein is a method for testing a vaccine, wherein the method includes contacting a sample containing the vaccine, such as a SUDV or EBOV GP immunogen, with a disclosed antibody under conditions sufficient for formation of an immune complex, and detecting the immune complex. Detection of the immune complex confirms that the SUDV or EBOV GP vaccine includes the epitope to which the 316L, 380L, 291S, 545S, 523S, 573S, 541S, 294S, 241S, 354S, 233S, 503S, 203S, 315S, 586S, 377S, 528S, 246S, 338S, 335S, 285S, 382S, 365S, 405S, 102S, 578S, 191L, 206L, 231L, 232L, 310L, 314L, 315L or 396L antibody binds. In one example, the detection of the immune complex in the sample indicates that a vaccine component, such as a SUDV or EBOV GP immunogen assumes a conformation capable of binding the antibody.

In one aspect, the antibody is directly labeled with a detectable marker. In another aspect, the antibody that binds SUDV or EBOV GP (the first antibody) is unlabeled and a second antibody or other molecule that can bind the antibody that binds the first antibody is utilized for detection. As is well known to one of skill in the art, a second antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody, antigen binding fragment or secondary antibody are described above, and include various enzymes. prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

### F. Kits

Kits are also provided. For example, kits for treating a subject with a SUDV or EBOV infection, or for detecting SUDV or EBOV GP in a sample or in a subject. The kits will typically include a disclosed SUDV or EBOV GP-specific antibody, bispecific antibody, or a nucleic acid molecule(s) or vector(s) encoding such molecules, or compositions including such molecules. More than one of the disclosed SUDV or EBOV GP-specific antibody, bispecific antibody, conjugate, or nucleic acid molecule or vector encoding such molecules, or compositions including such molecules can be included in the kit.

In one aspect, the kit is a diagnostic kit and includes an immunoassay. Although the details of the immunoassays may vary with the particular format employed, the method of detecting SUDV or EBOV GP in a biological sample generally includes the steps of contacting the biological sample with an antibody which specifically reacts, under conditions sufficient to form an immune complex, to SUDV or EBOV GP. The antibody is allowed to specifically bind under immunologically reactive conditions to form an immune complex, and the presence of the immune complex (bound antibody) is detected directly or indirectly.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, bispecific antibodies, conjugates, nucleic acid molecules, vectors, or compositions. In several aspects the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of the antibodies, bispecific antibodies, conjugates, nucleic acid molecules, or compositions included in the kit. The package insert typically includes instructions customarily included in commercial packages of therapeutic products that contain information about the indications, usage, dosage, administration, contraindications and/or warnings concerning the use of such therapeutic products. The instructional materials may be written, in an electronic form or may be visual. The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

### EXAMPLES

The following examples are provided to illustrate particular features of certain aspects of the disclosure, but the scope of the claims should not be limited to those features exemplified.

### Example 1: Characterization of Antibodies 316L and 380L

The 316L and 380L variable domains were isolated through nested PCR of the heavy and light chain immunoglobulin genes from GP-probe+, single-cell-sorted B-cells from NHP PBMC samples. The nucleotide and amino acid sequences of the VH and VL domains of 316L and 380L are set forth herein as SEQ ID NOs: 1-4 (316L) and SEQ ID NOs: 5-8 (380L).

### Binding to Ebola GP

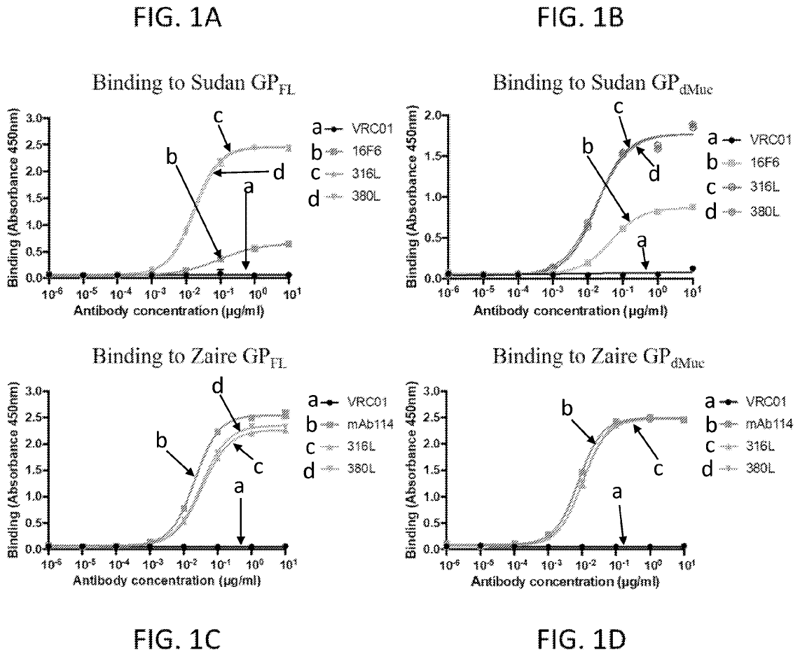

Binding of 316L and 380L to two different forms of Sudan GP and Zaire GP, full-length (FL) and mucin domain-deleted (dMuc) GP, was tested by ELISA. Antibodies 16F6 and mAb114, which bind SUDV GP and EBOV GP, respectively, were included as controls. Nonlinear polynomial curve fit of the ELISA optical density (OD) values for each antibody dilution were performed to determine the effective concentration for 50% binding (EC₅₀). As shown in FIGS. 1A-1D and Table 1, 316L and 380L exhibited binding to both forms of Sudan GP and both forms of Zaire GP.

TABLE 1
50% binding titers

| | EC₅₀ (ng/ml) | | | |
|---|---|---|---|---|
| | Sudan | | Zaire | |
| mAb | GP_{FL} | GP_{dMuc} | GP_{FL} | GP_{dMuc} |
| 16F6 | 68.0 ± 37 | 55.76 ± 34 | ND* | ND |
| mAb114 | ND | ND | 23.9 ± 4.1 | 9.04 ± 1.7 |
| 316L | 17.5 ± 2.2 | 27.2 ± 15.3 | 44.1 ± 14.1 | 12.4 ± 3.0 |
| 380L | 19.5 ± 1.6 | 23.8 ± 15.9 | 39.8 ± 10.7 | 12.0 ± 2.3 |

*ND, not determined.

### In Vitro Neutralization

The neutralizing ability of 316L and 380L against SUDV GP- and EBOV GP-pseudotyped lentiviral vectors was determined by microneutralization assay in vitro. mAbs were pre-incubated with the lentiviral vectors prior to being added to HEK293 cells in a 96-well format. Percent inhibition was calculated relative to infection in the absence of mAb (FIGS. 2A-2B). The 50% inhibitory concentrations (IC₅₀) are shown in Table 2.

TABLE 2
50% inhibitory titers

IC₅₀ (μg/ml)

| mAb | SUDV GP | EBOV GP |
|---|---|---|
| 16F6 | 0.068 ± 0.037 | ND* |
| mAb114 | ND | 0.152 ± 0.001 |
| 316L | 0.284 ± 0.014 | 1.772 ± 0.215 |
| 380L | 0.395 ± 0.168 | 0.756 ± 0.152 |

*ND, not determined.

### Kinetics of Binding to Ebola GP

Fabs generated from 316L and 380L were evaluated for binding to Sudan GP_{FL} (GP(S)FL), GP_{dMuc} (GP(S)dMuc) and GP_{THL} (GP(S)THL) at pH 7.4 and pH 5.3, and to Zaire GPH. (GP(Z)FL) and GP_{dMuc} (GP(Z)dMuc) at pH 7.4 by BLI. The affinity constants (K_{D}) for Fab binding to each protein are shown in Table 3.

TABLE 3
KD binding kinetics determined by BLI

| | | 316L | | | 380L | | |
|---|---|---|---|---|---|---|---|
| GP version | pH | K_{D} (M) | Kₒₙ (1/Ms) | K_{off} (1/s) | K_{D} (M) | Kₒₙ (1/Ms) | K_{off} (1/s) |
| GP(S)FL | 7.4 | 1.16 × 10⁻⁹ | 1.35 × 10⁵ | 1.57 × 10⁻⁴ | 2.85 × 10⁻⁹ | 1.01 × 10⁵ | 2.90 × 10⁻⁴ |
| | 5.3 | 1.26 × 10⁻⁹ | 1.10 × 10⁵ | 1.39 × 10⁻⁴ | 3.87 × 10⁻⁹ | 0.842 × 10⁵ | 3.26 × 10⁻⁴ |
| GP(S)dMuc | 7.4 | 0.372 × 10⁻⁹ | 1.45 × 10⁵ | 0.538 × 10⁻⁴ | 0.567 × 10⁻⁹ | 1.68 × 10⁵ | 0.954 × 10⁻⁴ |
| | 5.3 | 0.46 × 10⁻⁹ | 1.12 × 10⁵ | 0.526 × 10⁻⁴ | 0.64 × 10⁻⁹ | 1.72 × 10⁵ | 1.09 × 10⁻⁴ |
| GP(S)THL | 7.4 | 1.16 × 10⁻⁹ | 3.72 × 10⁵ | 4.33 × 10⁻⁴ | ND | ND | ND |
| | 5.3 | 2.05 × 10⁻⁹ | 3.87 × 10⁵ | 7.93 × 10⁻⁴ | ND | ND | ND |
| GP(Z)FL | 7.4 | 38.1 × 10⁻⁹ | 0.69 × 10⁵ | 26.3 × 10⁻⁴ | 19.8 × 10⁻⁹ | 0.63 × 10⁵ | 14.8 × 10⁻⁴ |
| GP(Z)dMuc | 7.4 | 13.3 × 10⁻⁹ | 1.15 × 10⁵ | 15.0 × 10⁻⁴ | 6.64 × 10⁻⁹ | 1.18 × 10⁵ | 7.78 × 10⁻⁴ |

### Blocking of NPC1-dC Binding to Sudan GP_{THL}

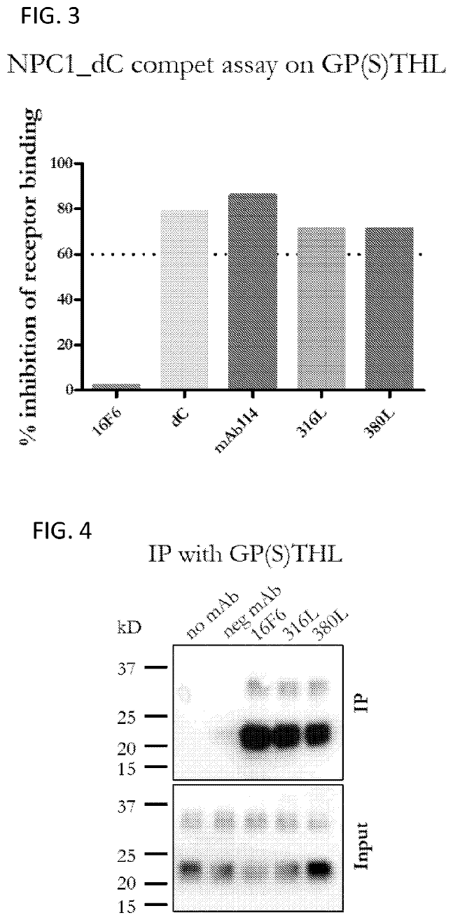

After Ebola virus enters into host cells, the GP needs to be cleaved by endosomal cathepsin to expose its receptor binding domain (RBD), resulting in GP_{THL}, which is recognized by its corresponding receptor, NPC1 (Misasi et al., Science 351(6279):1343-1346, 2016). Given the importance of GP_{T}HL recognition by NPC1, the ability of the 316L and 380L mAbs to block NPC1-dC binding to Sudan GP_{THL} was tested by BLI. BLI sensors immobilized with Sudan GP_{THL} were incubated with 316L mAb, 380L mAb or control mAb/reagent prior to incubation with NPC1-dC. As shown in FIG. 3, mAb 316L blocked approximately 71.18% NPC1-dC binding to GP(S)THL, while mAb 380L mAb blocked approximately 71.18% NPC1-dC binding to GP(S)THL. To directly validate the binding of 316L and 380L to GP(S)THL, an immunoprecipitation assay was performed (FIG. 4). These results indicate that 316L and 380L may mediate neutralization by blocking the recognition of cleaved GP_{THL} by its receptor NPC1. Negative control antibody 16F6, which binds to the base region of Sudan GP, showed no blocking of NPC1-dC binding to GP(S)THL.

### Gross Epitope by Competition Group Analysis

By assessing how 316L and 380L compete with previously characterized mAbs, gross epitopes can be determined. Competition class was determined using BLI. Given that 316L and 380L bind to both Zaire GP and Sudan GP (as determined by ELISA assay), the competition group analysis was performed on both Zaire and Sudan glycoproteins. Briefly, biosensors were loaded with purified mucin domain-deleted Zaire GP or Sudan GP. The competitor mAb (the mAb determining the class or gross epitope) was then allowed to bind to the antigen and the degree of binding was recorded. The analyte mAb was then allowed to bind and the degree of binding was recorded. Percent inhibition of the binding of the analyte was calculated as follows:

$$\% inhibition = 100 ( 1 - \frac{signal of analyte binding in the presence of competitor}{signal of analyte binding in the absence of competitor} )$$

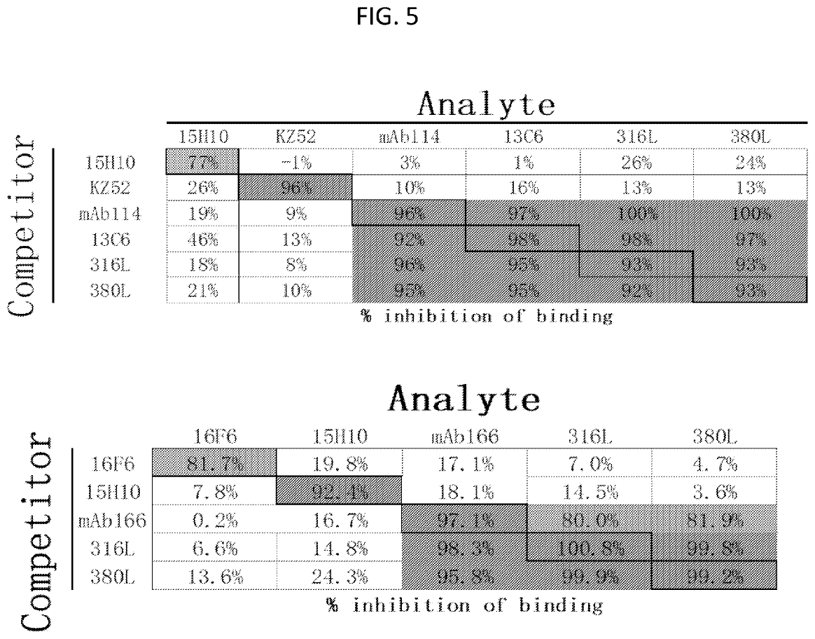

The results using Zaire GP indicated that 316L and 380L are in the same competition class as mAb114 (which binds at the RBS of the GP1 core) and 13C6 (which binds at the glycan cap of GP) (FIG. 5) (Misasi et al., Science 351(6279):1343-1346, 2016; Lee et al., Nature 454(7201):177-172, 2008). The assay performed using Sudan GP showed that 316L and 380L were in the same competition class as mAb166, which binds at the glycan cap of GP (FIG. 5). Given that 13C6 and mAb166 are glycan cap binders that do not bind to thermolysin cleaved GP, and 316L and 380L bind like mAb114, these results indicate that the 316L and 380L epitopes are in a mAb114-like location.

### Evaluation of 316L and 380L Epitopes by Transmission Electronic Microscopy

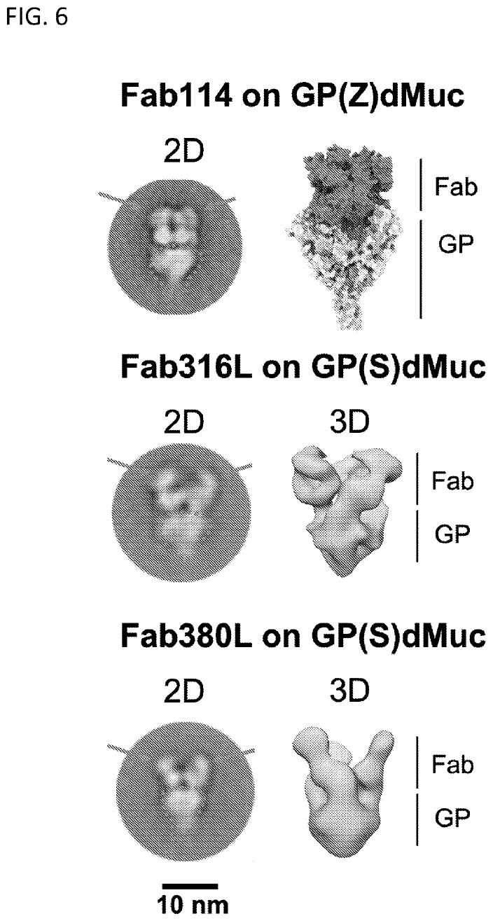

Mucin domain-deleted Sudan GP was incubated with molar excess Fab generated from 316L and 380L mAbs to form complexes that were evaluated by negative-stain transmission electron microscopy. Class averages were generated from single particle image analysis. Within the set of class averages, classes were identified that showed the binding of both Fabs to GP in a manner similar to that seen for Fab generated from mAb114, indicating that the binding site on GP is likely to be very similar. 3D class averages also were generated for analysis of detailed binding sites of 316L and 380L to Sudan GP_{dMuc} (FIG. 6).

### In Vivo Efficacy Against Lethal Sudan Gulu Challenge of Macaques

The macaque model of SUDV infection is the standard for assessing vaccines, antibodies and antivirals against EVD. Using this model, macaques are challenged with a target dose of 1000 PFU early passage SUDV Gulu or Boneface. This virus dose is between 50-90% lethal in naïve macaques and death occurs between 6-12 days after virus challenge. Challenges are performed at USAMRIID, where >20 historical controls have been infected. The use of historical controls in challenge studies allows statistically significant determination of treatment efficacy using small treatment groups of 3-4 macaques per group and a single or fewer untreated control animals.

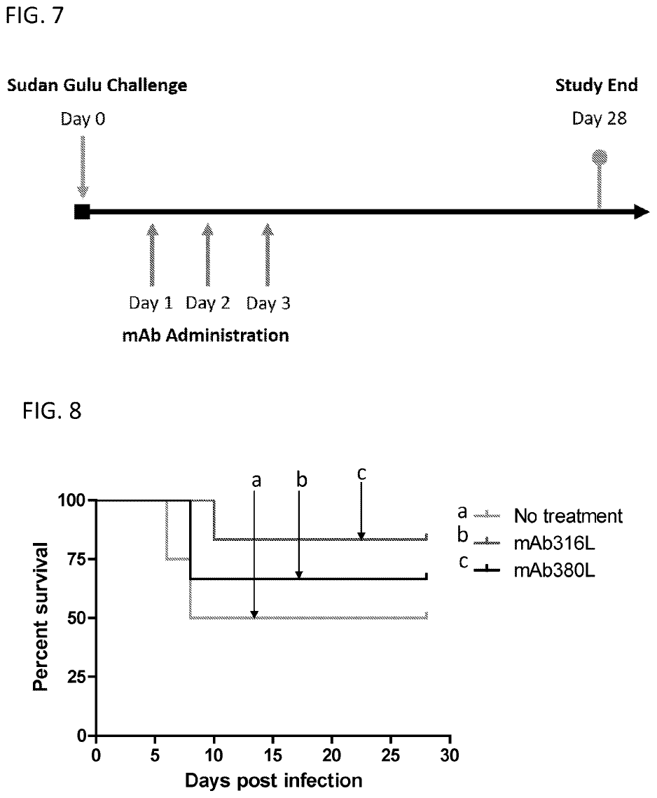

In this study, 316L or 380L was administered to macaques by three intravenous (IV) injections at 24-hour intervals at a dose of 50 mg/kg/dose beginning 24 hours after lethal challenge (1000 PFU) with Sudan Gulu strain (Table 4 and FIG. 7).

TABLE 4
Dosage of mAb administered intravenously to macaques
for in vivo efficacy studies (number (#) of animals
were combined from two independent experiments)

| # of | Antibody | | |
|---|---|---|---|
| Animals | Administered | Dosage | Administrations |
| 6 | 316L | 50 mg/kg/dose | 3 |
| 3 | 380L | 50 mg/kg/dose | 3 |
| 4 | none | N/A | none |

All animals in the group administered 316L survived SUDV challenge to at least 28 days post-infection, while 2 of 3 animals administered 380L survived (FIG. 8). In comparison, 2 of 4 control animals survived the challenge to at least 28 days.

Administration of 316L in Combination with 523S

316L±523S antibody mixtures were administrated to macaques by three intravenous (IV) injections at 24-hour intervals at a dosage of 50 mg/kg/dose beginning 24 hours after lethal challenge (100 PFU) with Sudan Gulu strain. One ratio of 316L to 523S was tested: 50% 316L:50% 523S (Table 5).

TABLE 5
Dosage of mAb administered intravenously
to macaques for in vivo efficacy studies

| # of | Antibody | | | Admin- |
|---|---|---|---|---|
| Animals | Administered | 316L:523S | Dosage | istrations |
| 3 | 316L + 523S | 50%:50% | 50 mg/kg/dose | 3 |
| 3 | none | N/A | N/A | none |

As shown in FIG. 9, three out of the three animals in the group administered 316L±523S survived SUDV challenge to at least 28 days post-infection, whereas all untreated control animals succumbed to infection.

### Example 2: Characterization of Antibodies 291S and 545S

The 291S and 545S variable domains were isolated through nested PCR of the heavy and light chain immunoglobulin genes from GP-probe+, single-cell-sorted B-cells from human PBMC samples. The nucleotide and amino acid sequences of the VH and VL domains of 291S and 545S are set forth herein as SEQ ID NOs: 9-12 (291S) and SEQ ID NOs: 13-16 (545S).

### Binding to Sudan GP

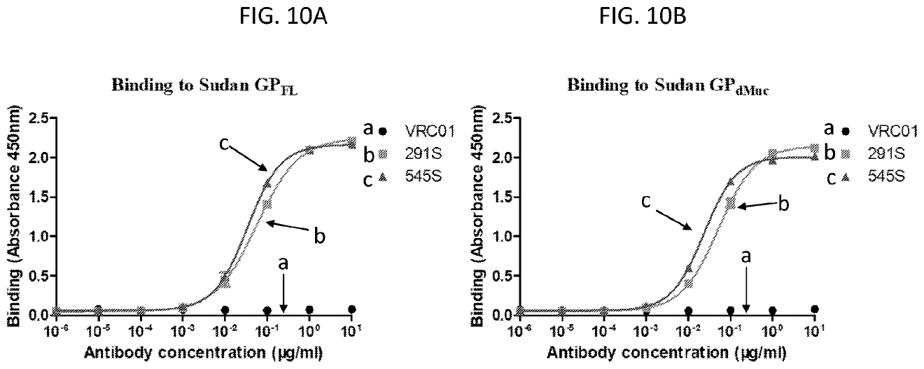

ELISA binding studies were performed by coating plates with Sudan GP produced in Expi293F cells and diluted in bicarbonate buffer. Two forms of GP from Sudan virus were tested: full length GP (FL) and mucin domain-deleted GP (dMuc). Nonlinear polynomial curve fit of the ELISA OD values for each mAb dilution were performed to determine the EC₅₀. 291S and 545S exhibited binding to both forms of GP (FIGS. 10A-10B and Table 6).

TABLE 6
50% binding titers (EC50, μg/ml)

Sudan

| mAb | GP_{FL} | GP_{dMuc} |
|---|---|---|
| 291S | 0.05652 | 0.05194 |
| 545S | 0.03376 | 0.02334 |

### In Vitro Neutralization

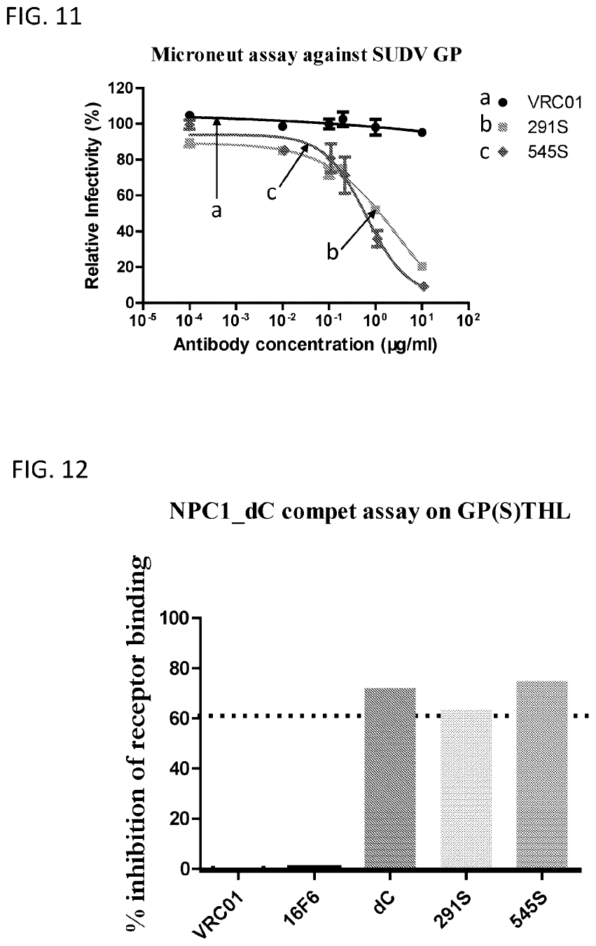

The neutralizing ability of 291S and 545S against SUDV GP-pseudotyped lentiviral vectors was determined by microneutralization assay in vitro. mAb was pre-incubated with the lentiviral vectors prior to being added to HEK293 cells in a 96-well format. Percent inhibition was calculated relative to infection in the absence of mAb (FIG. 11). The 50% inhibitory concentrations are shown in Table 7.

TABLE 7
50% Inhibitory titers (IC₅₀, μg/ml)

| mAb | SUDV GP |
|---|---|
| 291S | 2.893 |
| 545S | 0.6236 |

### Kinetics of Binding to Sudan GP

Fab generated from 291S and 545S were evaluated for binding to Sudan GP_{FL} and GP_{THL} at pH 7.4 and pH 5.3 by BLI. Affinity constants (K_{D}) are listed in Table 8.

TABLE 8
KD binding kinetics determined by BLI

| | | 291S | | | 545S | | |
|---|---|---|---|---|---|---|---|
| GP version | pH | K_{D} (M) | Kₒₙ (1/Ms) | K_{off} (1/s) | K_{D} (M) | Kₒₙ (1/Ms) | K_{off} (1/s) |
| GP(S)FL | 7.4 | 2.97 × 10⁻⁹ | 4.42 × 10⁴ | 1.31 × 10⁻⁴ | 1.26 × 10⁻⁹ | 9.92 × 10⁴ | 1.25 × 10⁻⁴ |
| | 5.3 | 4.27 × 10⁻⁹ | 2.57 × 10⁴ | 1.10 × 10⁻⁴ | 1.97 × 10⁻⁹ | 7.43 × 10⁴ | 1.47 × 10⁻⁴ |
| GP(S)THL | 7.4 | 0.19 × 10⁻⁹ | 2.64 × 10⁵ | 0.51 × 10⁻⁴ | 1.60 × 10⁻⁹ | 3.27 × 10⁵ | 5.25 × 10⁻⁴ |
| | 5.3 | 0.67 × 10⁻⁹ | 2.48 × 10⁵ | 1.66 × 10⁻⁴ | 1.55 × 10⁻⁹ | 2.91 × 10⁵ | 4.51 × 10⁻⁴ |

### Blocking of NPC1-dC Binding to Sudan GP_{THL}

The ability of 291S and 545S mAbs to block NPC1-dC binding to Sudan GP_{THL} was tested by BLI. BLI sensors immobilized with Sudan GP_{THL} were incubated with 291S mAb, 545S mAb or control mAb/reagent prior to incubation with NPC1-dC. As shown in FIG. 12, 291S and 545S blocked approximately 64% and 75%, respectively, of NPC1-dC binding to GP(S)THL.

### Gross Epitope by Competition Group Analysis

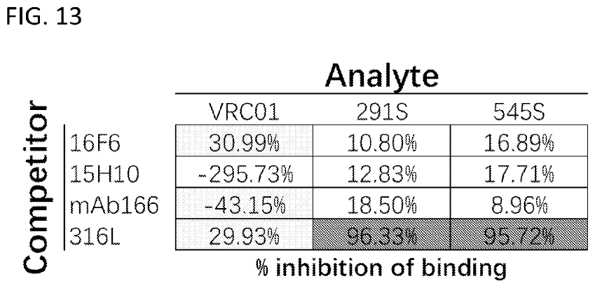

Gross epitope determination via BLI competition assay was determined as described in Example 1, except that biosensors were loaded only with purified mucin domain-deleted Sudan GP. The results showed that 291S and 545S are in the same competition class as 316L, which binds at the RBS of the GP1 core (FIG. 13), indicating that the 291S and 545S epitopes are in a mAb114-like location.

### Evaluation of Epitope by Transmission Electron Microscopy

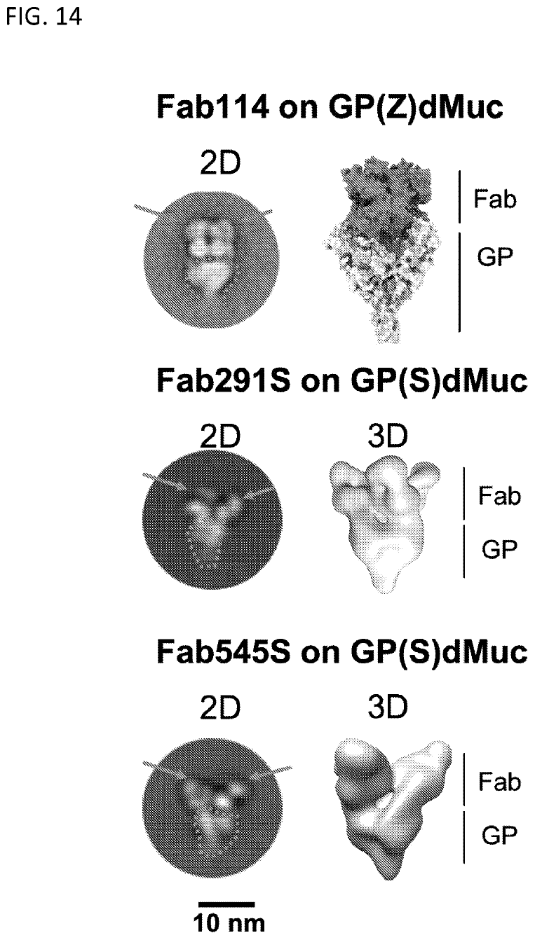

Mucin domain-deleted Sudan GP was incubated with molar excess Fab generated from 291S mAb or 545S mAb to form complexes, which were evaluated by negative-stain transmission electron microscopy. Class averages were generated from single particle image analysis. Within the set of class averages, classes were identified that showed the binding of 291S and 545S Fab to GP in a manner similar to that seen for Fab generated from mAb114 or 316L, indicating that their binding sites on GP are likely very similar. 3D class averages were also generated for analysis of detailed binding sites of 291S and 545S to Sudan GP_{dMuc} (FIG. 14).

In Vivo Administration of 545S in Combination with 523S

545S±523S antibody mixtures were administrated to macaques by three IV injections at 24-hour intervals at a dosage of 50 mg/kg/dose beginning 24 hours after lethal challenge (100 PFU) with Sudan Gulu strain (see study schematic shown in FIG. 7). One ratio of 545S to 523S was tested: 50% 545S:50% 523S (Table 9).

TABLE 9
Dosage of mAb administered intravenously
to macaques for in vivo efficacy studies

| # of | Antibody | | | |
|---|---|---|---|---|
| Animals | Administered | 545S:523S | Dosage | Administrations |
| 3 | 545S + 523S | 50%:50% | 50 mg/kg/dose | 3 |
| 1 | none | N/A | N/A | none |

Three out of the three animals in the group administered 545S±523S survived SUDV challenge to at least 28 days post-infection, whereas all untreated control animals succumbed to infection (FIG. 15).

### Example 3: Characterization of 523S, 573S, 541S, 294S, 241S, 354S, 233S and 503S

Antibody 523S, 573S, 541S, 294S, 241S, 354S, 233S and 503S variable domains were isolated through nested PCR of the heavy and light chain immunoglobulin genes from GP-probe+, single-cell-sorted B-cells from human PBMC samples. The nucleotide and amino acid sequences of the VH and VL domains of each antibody are set forth herein as follows:

| mAb | SEQ ID NOs |
|---|---|
| 523S | 17-20 |
| 573S | 21-24 |
| 541S | 25-28 |
| 294S | 29-32 |
| 241S | 33-36 |
| 354S | 37-40 |
| 233S | 41-44 |
| 503S | 45-48 |

### Binding to Sudan GP

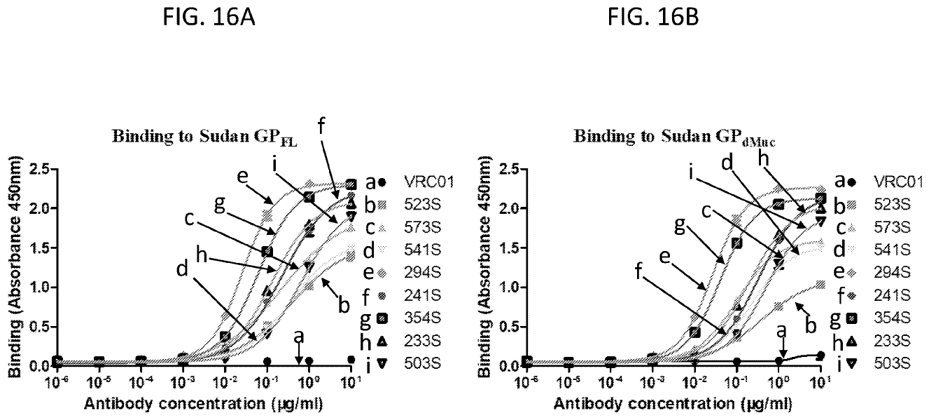

Standard ELISA binding studies were performed by coating plates with Sudan GP (GP_{FL} and GP_{dMuc}) produced in Expi293F cells and diluted in bicarbonate buffer. Nonlinear polynomial curve fit of the ELISA OD values for each mAb dilution were performed to determine the EC₅₀. All mAbs showed binding to both forms of GP (FIG. 16 and Table 10).

TABLE 10
Binding titers (EC50, μg/ml)

Sudan

| mAb | GP_{FL} | GP_{dMuc} |
|---|---|---|
| 523S | 0.3710 | 0.3782 |
| 573S | 0.2210 | 0.1008 |
| 541S | 0.3679 | 0.2298 |
| 294S | 0.0258 | 0.02661 |
| 241S | 0.2318 | 0.3309 |
| 354S | 0.0613 | 0.04042 |
| 233S | 0.1329 | 0.1953 |
| 503S | 0.6402 | 0.4989 |

### In Vitro Neutralization

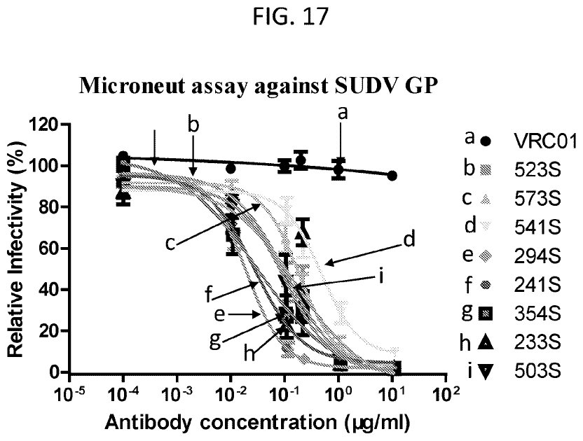

The neutralizing ability of 523S, 573S, 541S, 294S, 241S, 354S, 233S and 503S against SUDV GP-pseudotyped lentiviral vectors was determined by microneutralization assay in vitro. mAb was pre-incubated with the lentiviral vectors prior to being added to HEK293 cells in a 96-well format. Percent inhibition was calculated relative to infection in the absence of mAb (FIG. 17). The 50% inhibitory concentrations are shown in Table 11.

TABLE 11
50% Inhibitory titers (IC₅₀, μg/ml)

| mAb | SUDV GP |
|---|---|
| 523S | 0.1082 |
| 573S | 0.1879 |
| 541S | 0.4778 |
| 294S | 0.01917 |
| 241S | 0.02751 |
| 354S | 0.02997 |
| 233S | 0.1671 |
| 503S | 0.1025 |

### Kinetics of Binding to Sudan GP

Fabs generated from 523S, 573S, 541S, 294S, 241S, 354S, 233S and 503S were evaluated for binding to Sudan GP_{FL} at pH 7.4 and 5.3 by BLI. The results are shown in Table 12.

TABLE 12
K_{D} binding kinetics determined by BLI

| | K_{D} binding to GP(S)FL, pH 7.4 | | | K_{D} binding to GP(S)FL, pH 5.3 | | |
|---|---|---|---|---|---|---|
| Fab | K_{D} (M) | Kₒₙ (1/Ms) | K_{off} (1/s) | K_{D} (M) | Kₒₙ (1/Ms) | K_{off} (1/s) |
| 523S | 1.61 × 10⁻⁹ | 0.919 × 10⁵ | 1.48 × 10⁻⁴ | 1.58 × 10⁻⁹ | 0.654 × 10⁵ | 1.03 × 10⁻⁴ |
| 573S | 24.1 × 10⁻⁹ | 0.105 × 10⁵ | 2.52 × 10⁻⁴ | 9.27 × 10⁻⁹ | 0.113 × 10⁵ | 1.05 × 10⁻⁴ |
| 541S | 39.5 × 10⁻⁹ | 0.122 × 10⁵ | 4.82 × 10⁻⁴ | 44.3 × 10⁻⁹ | 0.0726 × 10⁵ | 3.22 × 10⁻⁴ |
| 294S | 4.3 × 10⁻⁹ | 3.14 × 10⁵ | 13.5 × 10⁻⁴ | 2.18 × 10⁻⁹ | 1.73 × 10⁵ | 3.77 × 10⁻⁴ |
| 241S | 3.47 × 10⁻⁹ | 1.27 × 10⁵ | 4.39 × 10⁻⁴ | 6.36 × 10⁻⁹ | 0.682 × 10⁵ | 4.33 × 10⁻⁴ |
| 354S | 8.89 × 10⁻⁹ | 0.856 × 10⁵ | 7.60 × 10⁻⁴ | 6.12 × 10⁻⁹ | 0.727 × 10⁵ | 4.45 × 10⁻⁴ |
| 233S | 7.53 × 10⁻⁹ | 0.721 × 10⁵ | 5.43 × 10⁻⁴ | 7.56 × 10⁻⁹ | 0.473 × 10⁵ | 3.58 × 10⁻⁴ |
| 503S | 19.5 × 10⁻⁹ | 0.568 × 10⁵ | 11.1 × 10⁻⁴ | 6.58 × 10⁻⁹ | 0.567 × 10⁵ | 3.73 × 10⁻⁴ |

### Evaluation of Gross Epitope by Competition Group Analysis

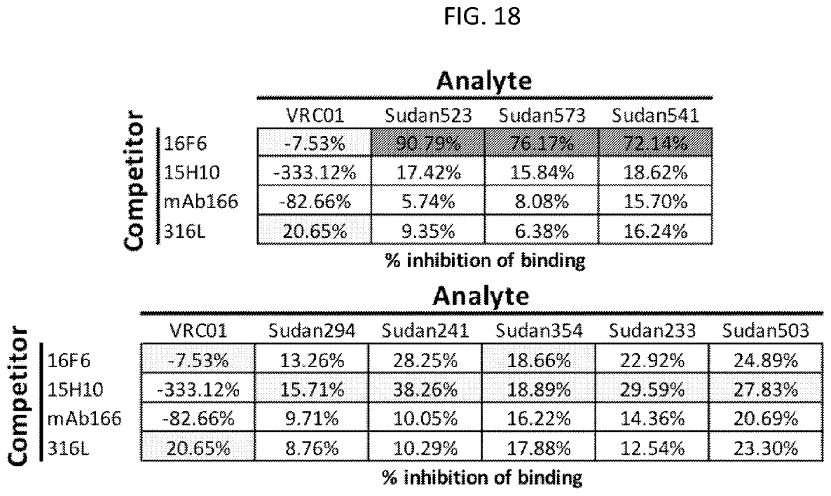

Gross epitope determination via BLI competition assay was determined as described in Example 1, except that biosensors were loaded only with purified mucin domain-deleted Sudan GP. The results showed that antibodies 523S, 573S and 541S are in the same competition class as 16F6 (FIG. 18), which binds at the base region of GP, indicating that the epitopes of these antibodies are in a 16F6-like location. Antibodies 294S, 241S, 354S, 233S and 503S were not in any competition group, indicating their epitopes are unique relative to the antibody competitors used in the assay.

### Evaluation of Epitope by Transmission Electron Microscopy

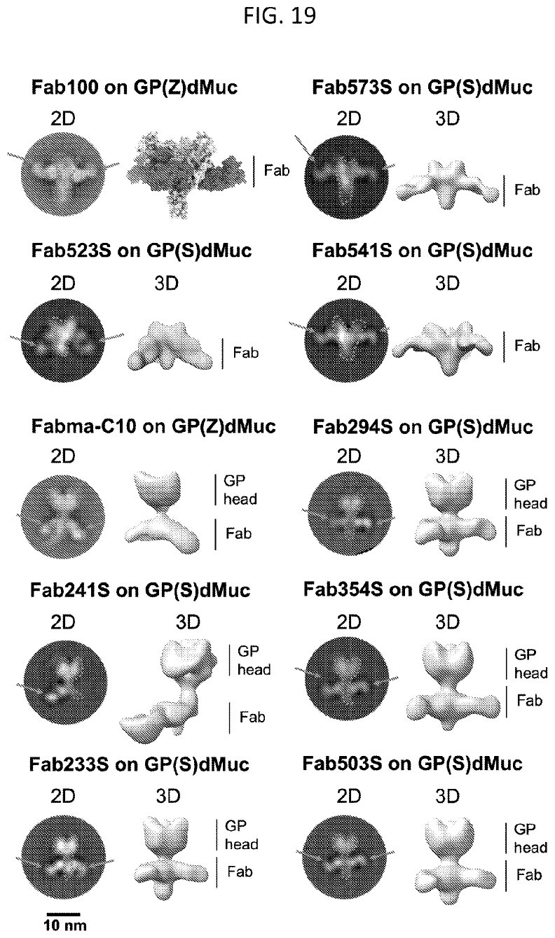

Mucin domain-deleted Sudan GP was incubated with molar excess Fab generated from 523S, 573S, 541S, 294S, 241S, 354S, 233S and 503S mAbs to form complexes that were evaluated with negative-stain transmission electron microscopy. Class averages were generated from single particle image analysis. The results are shown in FIG. 19. Within the set of class averages, classes were identified that showed the binding of 523S, 573S and 541S Fabs to GP in a manner similar to that seen for Fab generated from mAb100 or 16F6. In addition, classes were identified that showed the binding of 294S, 241S, 354S, 233S and 503S Fabs to GP in a manner similar to that seen for Fab generated from ma-C10, which binds to the MPER/HR2 region of GP. Thus, the binding sites of 523S, 573S and 541S are likely to be very similar to the binding sites of mAb100 and 16F6, while the bindings sites of 294S, 241S, 354S, 233S and 503S are likely to be very similar to the binding site of ma-C10. FIG. 19 also shows 3D class averages generated for analysis of detailed binding sites of 523S, 573S, 541S, 294S, 241S, 354S, 233S and 503S to Sudan GP_{dMuc}.

### Example 4: Characterization of Antibodies 203S, 315S, 586S, 377S, 528S, 246S, 338S, 335S, 285S, 382S, 365S, 405S, 102S and 578S

The 203S, 315S, 586S, 377S, 528S, 246S, 338S, 335S, 285S, 382S, 365S, 405S, 102S and 578S variable domains were isolated through nested PCR of the heavy and light chain immunoglobulin genes from GP-probe+, single-cell-sorted B-cells from human PBMC samples. The nucleotide and amino acid sequences of the VH and VL domains of antibodies 203S, 315S, 586S, 377S, 528S, 246S, 338S, 335S, 285S, 382S, 365S, 405S, 102S and 578S are set forth herein as follows:

| mAb | SEQ ID NOs: |
|---|---|
| 203S | 49-52 |
| 315S | 53-56 |
| 586S | 57-60 |
| 377S | 61-64 |
| 528S | 65-68 |
| 246S | 69-72 |
| 338S | 73-76 |
| 335S | 77-80 |
| 285S | 81-84 |
| 382S | 85-88 |
| 365S | 89-92 |
| 405S | 93-96 |
| 102S | 97-100 |
| 578S | 101-104 |

### Binding to Sudan GP

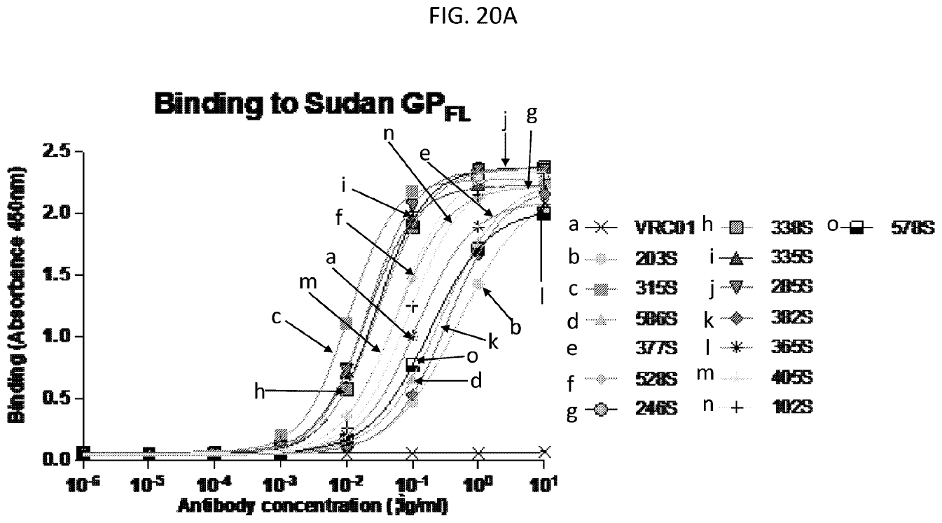

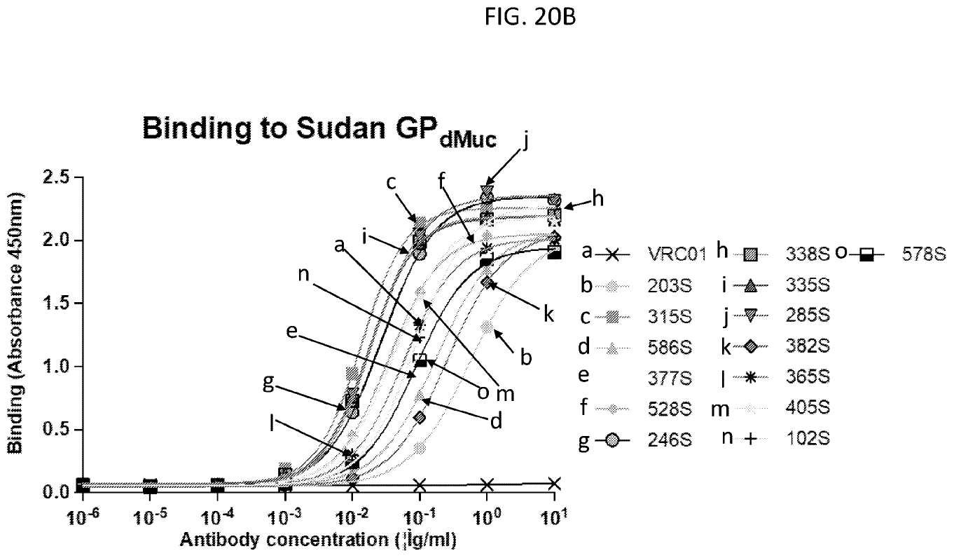

Standard ELISA binding studies were performed by coating plates with Sudan GP (GP_{FL} and GP_{dMuc}) produced in Expi293F cells and diluted in bicarbonate buffer. Nonlinear polynomial curve fit of the ELISA OD values for each mAb dilution were performed to determine the EC₅₀. The results demonstrated that antibodies 203S, 315S, 586S, 377S, 528S, 246S, 338S, 335S, 285S, 382S, 365S, 405S, 102S and 578S all bind to both forms of GP (FIG. 20 and Table 13).

TABLE 13
50% binding titers (EC50, μg/ml)

| | |
|---|---|
| Sudan | Sudan |

| mAb | GP_{FL} | GP_{dMuc} | mAb | GP_{FL} | GP_{dMuc} |
|---|---|---|---|---|---|
| 203S | 0.50 | 0.57 | 335S | 0.02 | 0.02 |
| 315S | 0.01 | 0.01 | 285S | 0.02 | 0.02 |
| 586S | 0.28 | 0.18 | 382S | 0.35 | 0.26 |
| 377S | 0.14 | 0.86 | 365S | 0.11 | 0.06 |
| 528S | 0.05 | 0.03 | 405S | 0.06 | 0.04 |
| 246S | 0.03 | 0.03 | 102S | 0.09 | 0.09 |
| 338S | 0.03 | 0.02 | 578S | 0.18 | 0.09 |

### In Vitro Neutralization

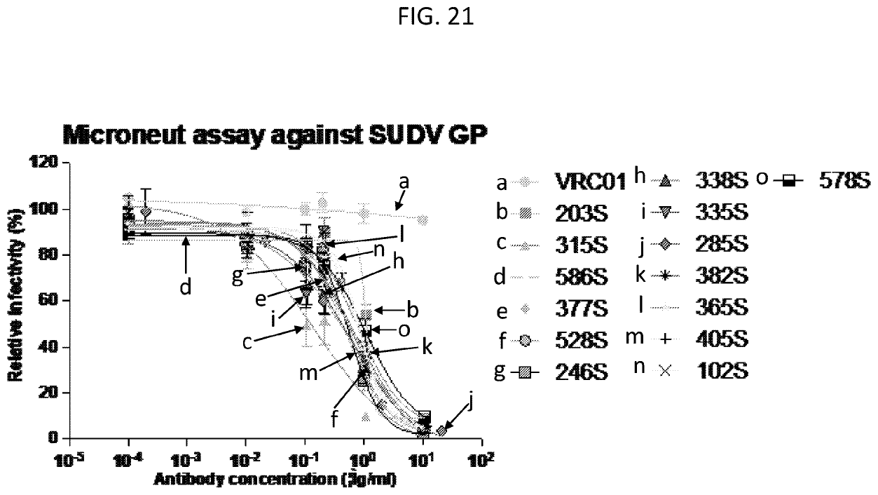

The neutralizing ability of the 203S, 315S, 586S, 377S, 528S, 246S, 338S, 335S, 285S, 382S, 365S, 405S, 102S and 578S antibodies against SUDV GP-pseudotyped lentiviral vectors was determined by microneutralization assay in vitro. mAb was pre-incubated with the lentiviral vectors prior to being added to HEK293 cells in a 96-well format. Percent inhibition was calculated relative to infection in the absence of mAb (FIG. 21). The 50% inhibitory concentrations are shown in Table 14.

TABLE 14
50% Inhibitory titers (IC₅₀, μg/ml)

| mAb | SUDV GP |
|---|---|
| 203S | 0.04 |
| 315S | 0.15 |
| 586S | 0.50 |
| 377S | 0.56 |
| 528S | 0.58 |
| 246S | 0.58 |
| 338S | 0.60 |
| 335S | 0.63 |
| 285S | 0.64 |
| 382S | 0.71 |
| 365S | 0.79 |
| 405S | 0.79 |
| 102S | 0.88 |
| 578S | 1.09 |

### Kinetics of Binding to Sudan GP

Fabs generated from 203S, 315S, 586S, 377S, 528S, 246S, 338S, 335S, 285S, 382S, 365S, 102S and 578S were evaluated for binding to Sudan GP_{FL} at pH 7.4 and 5.3 by BLI. The affinity constants (K_{D}) of each Fab at pH 7.4 and pH 5.3 are listed in Table 15.

TABLE 15
KD binding kinetics determined by BLI

| | K_{D} binding to GP(S)FL, pH 7.4 | | | K_{D} binding to GP(S)FL, pH 5.3 | | |
|---|---|---|---|---|---|---|
| Fab | K_{D} (M) | Kₒₙ (1/Ms) | K_{off} (1/s) | K_{D} (M) | Kₒₙ (1/Ms) | K_{off} (1/s) |
| 203S | 26.1 × 10⁻⁹ | 0.144 × 10⁵ | 3.76 × 10⁻⁴ | 118.0 × 10⁻⁹ | 0.0388 × 10⁵ | 4.60 × 10⁻⁴ |
| 315S | 8.37 × 10⁻⁹ | 0.362 × 10⁵ | 3.03 × 10⁻⁴ | 4.26 × 10⁻⁹ | 0.210 × 10⁵ | 0.893 × 10⁻⁴ |
| 586S | 37.1 × 10⁻⁹ | 0.149 × 10⁵ | 5.54 × 10⁻⁴ | 933.0 × 10⁻⁹ | 0.00249 × 10⁵ | 2.32 × 10⁻⁴ |
| 377S | 36.7 × 10⁻⁹ | 0.172 × 10⁵ | 6.31 × 10⁻⁴ | 63.7 × 10⁻⁹ | 0.0897 × 10⁵ | 5.71 × 10⁻⁴ |
| 528S | 9.11 × 10⁻⁹ | 0.425 × 10⁵ | 3.87 × 10⁻⁴ | 5.58 × 10⁻⁹ | 0.0833 × 10⁵ | 0.465 × 10⁻⁴ |
| 246S | 23.2 × 10⁻⁹ | 0.212 × 10⁵ | 4.93 × 10⁻⁴ | 34.2 × 10⁻⁹ | 0.112 × 10⁵ | 3.84 × 10⁻⁴ |
| 338S | 27.4 × 10⁻⁹ | 0.231 × 10⁵ | 6.32 × 10⁻⁴ | 23.7 × 10⁻⁹ | 0.176 × 10⁵ | 4.18 × 10⁻⁴ |
| 335S | 5.60 × 10⁻⁹ | 0.885 × 10⁵ | 4.96 × 10⁻⁴ | 12.60 × 10⁻⁹ | 0.278 × 10⁵ | 3.52 × 10⁻⁴ |
| 285S | 6.11 × 10⁻⁹ | 0.422 × 10⁵ | 2.58 × 10⁻⁴ | 18.2 × 10⁻⁹ | 0.168 × 10⁵ | 3.05 × 10⁻⁴ |
| 382S | 26.0 × 10⁻⁹ | 0.162 × 10⁵ | 4.22 × 10⁻⁴ | 31.3 × 10⁻⁹ | 0.145 × 10⁵ | 4.52 × 10⁻⁴ |
| 365S | 102.0 × 10⁻⁹ | 0.168 × 10⁵ | 17.1 × 10⁻⁴ | 57.7 × 10⁻⁹ | 0.628 × 10⁵ | 36.2 × 10⁻⁴ |
| 102S | 91.2 × 10⁻⁹ | 0.553 × 10⁵ | 50.4 × 10⁻⁴ | 9800 × 10⁻⁹ | 0.0121 × 10⁵ | 1190 × 10⁻⁴ |
| 578S | 97.1 × 10⁻⁹ | 0.042 × 10⁵ | 4.04 × 10⁻⁴ | 334.3 × 10⁻⁹ | 0.021 × 10⁵ | 6.95 × 10⁻⁴ |

### Evaluation of Gross Epitope by Competition Group Analysis

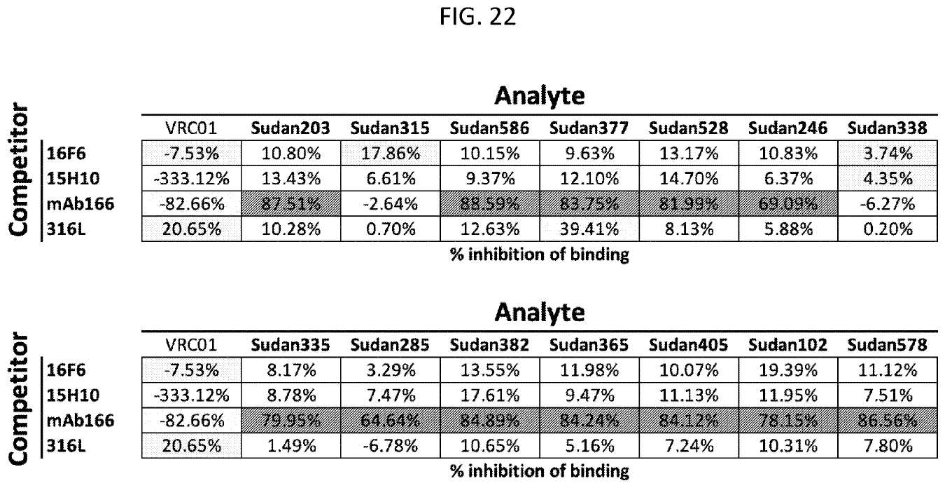

Gross epitope determination via BLI competition assay was determined as described in Example 1, except that biosensors were loaded only with purified mucin domain-deleted Sudan GP. As shown in FIG. 22, antibodies 203S, 586S, 377S, 528S, 246S, 335S, 285S, 382S, 365S, 405S, 102S and 578S are in the same competition class as mAb166, which binds at the glycan cap region of GP. These results indicate that the 203S, 586S, 377S, 528S, 246S, 335S, 285S, 382S, 365S, 405S, 102S and 578S epitopes are in a mAb166-like location. Antibodies 315S and 338S did not significantly compete with any of the competitor antibodies tested.

### Evaluation of Epitope by Transmission Electron Microscopy

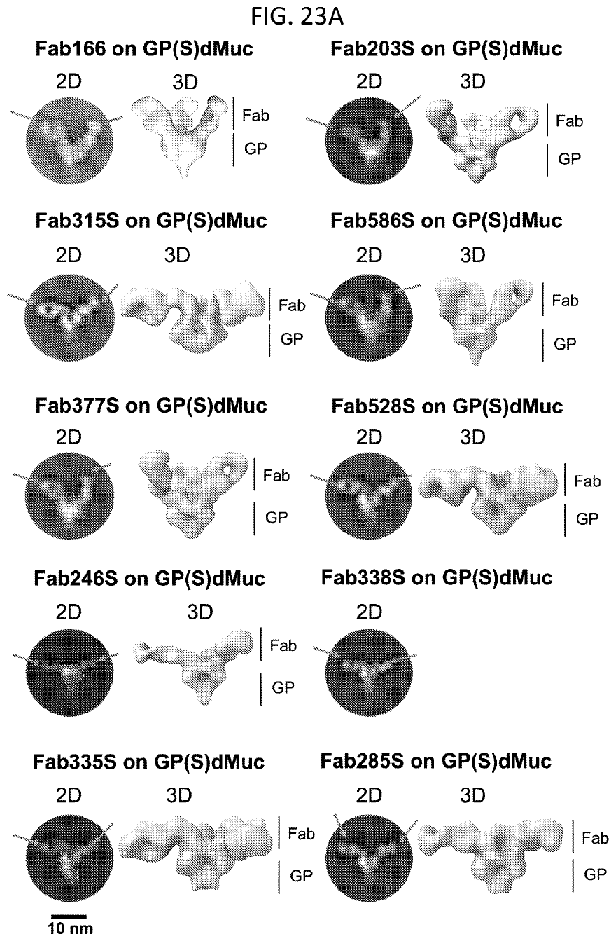

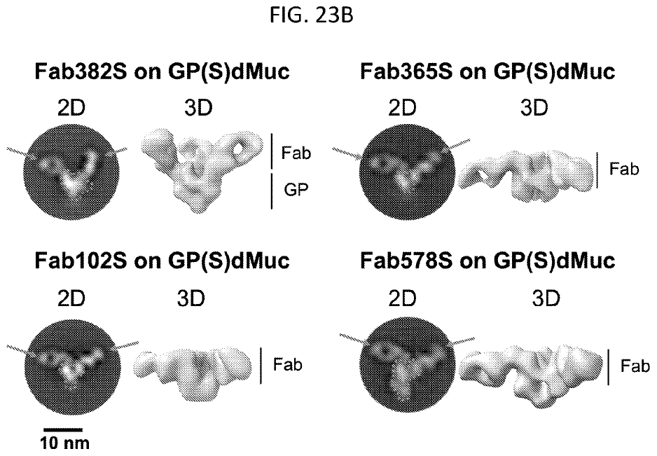

Mucin domain-deleted Sudan GP was incubated with molar excess Fab generated from the 203S, 315S, 586S, 377S, 528S, 246S, 338S, 335S, 285S, 382S, 365S, 102S and 578S mAbs to form complexes that were evaluated by negative-stain transmission electron microscopy. Class averages were generated from single particle image analysis. Within the set of class averages, classes were identified that showed the binding of all tested Fabs to GP in a manner similar to that seen for Fab generated from mAb166 indicating that the binding sites for these antibodies on GP are likely to be very similar (FIGS. 23A-23B). 3D class averages were also generated for analysis of detailed binding sites of 203S, 315S, 586S, 377S, 528S, 246S, 335S, 285S, 382S, 365S, 102S and 578S to Sudan GP_{dMuc} (FIGS. 23A-23B).

### Example 5: Characterization of Antibodies 191L, 206L, 231L, 232L, 310L, 314L, 315L and 396L

The 191L, 206L, 231L, 232L, 310L, 314L, 315L and 396L variable domains were isolated through nested PCR of the heavy and light chain immunoglobulin genes from GP-probe+, single-cell-sorted B-cells from NHP PBMC samples. The nucleotide and amino acid sequences of the VH and VL domains of antibodies 191L, 206L, 231L, 232L, 310L, 314L, 315L and 396L are set forth herein as follows:

| mAb | SEQ ID NOs: |
|---|---|
| 191L | 105-108 |
| 206L | 109-112 |
| 231L | 113-116 |
| 232L | 117-120 |
| 310L | 121-124 |
| 314L | 125-128 |
| 315L | 129-132 |
| 396L | 133-136 |

### Binding to Ebola GP

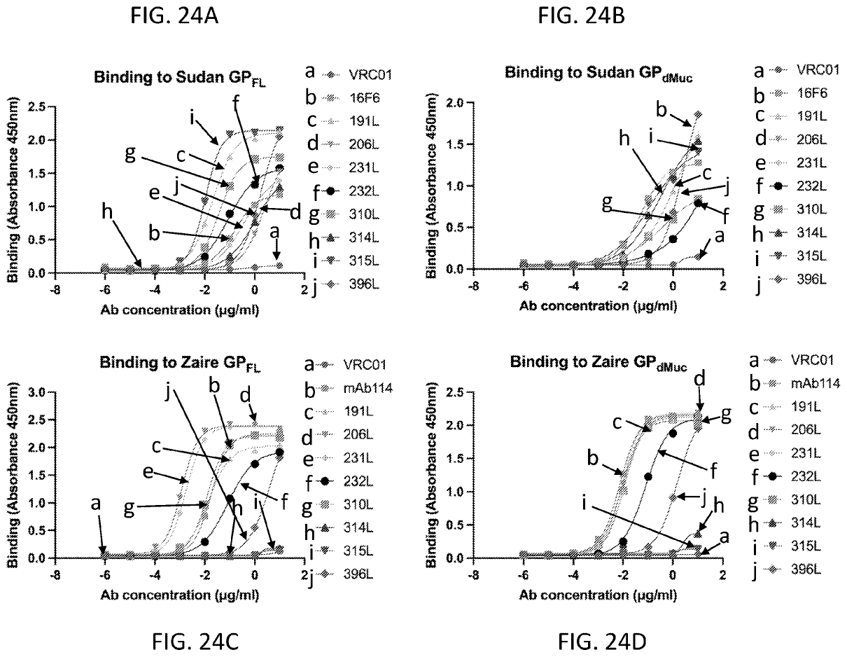

Standard ELISA binding studies were performed by coating plates with Sudan and Zaire GP (GP_{FL} and GP_{dMuc}) produced in Expi293F cells and diluted in bicarbonate buffer. Nonlinear polynomial curve fit of the ELISA OD values for each mAb dilution were performed to determine the EC₅₀. The results showed that antibodies 191L, 206L, 231L, 232L, 310L, 314L, 315L and 396L bind to both forms of Sudan GP and both forms of Zaire GP (FIG. 24 and Table 16).

TABLE 16
50% binding titers

| EC₅₀ (μg/ml) | |
|---|---|
| Sudan | Zaire |

| mAb | GP_{FL} | GP_{dMuc} | GP_{FL} | GP_{dMuc} |
|---|---|---|---|---|
| 16F6 | 0.16 | 0.05 | ND* | ND |
| mAb114 | ND | ND | 0.02 | 0.007 |
| 191L | 0.02 | 1.74 | 0.01 | 0.01 |
| 206L | 2.58 | 0.2 | 0.001 | 0.01 |
| 231L | 0.4 | 0.02 | 0.002 | 0.01 |
| 232L | 0.09 | >10 | 0.08 | 0.08 |
| 310L | 0.04 | 0.34 | 0.01 | 0.01 |
| 314L | 1.05 | 0.31 | 1.73 | 2.6 |
| 315L | 0.01 | 0.12 | 1.8 | 1.5 |
| 396L | 1.8 | 2.51 | 3.92 | 1.5 |

*ND, not determined.

### In Vitro Neutralization

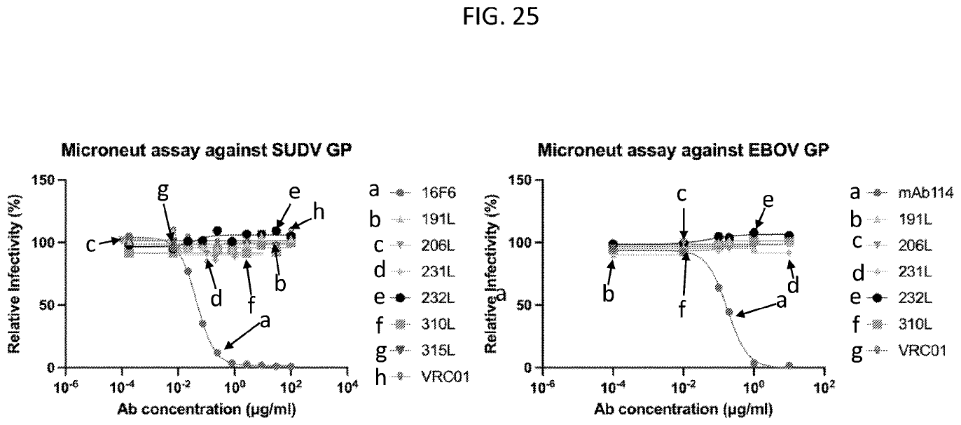

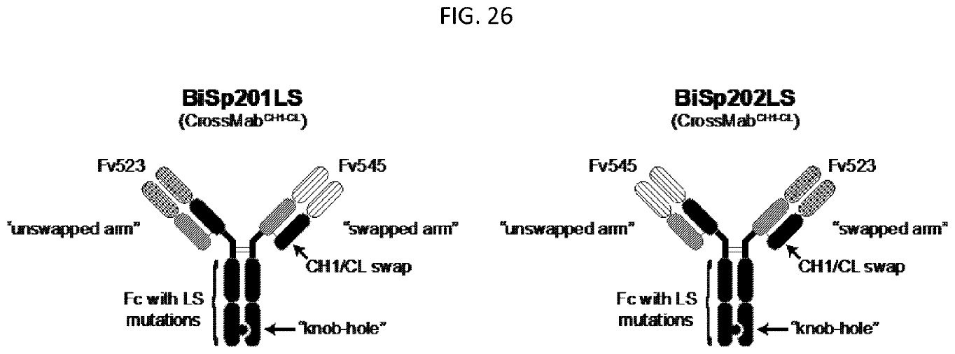

The neutralizing ability of 191L, 206L, 231L, 232L, 310L and 315L against Zaire GP- and Sudan GP-pseudotyped lentiviral vectors was determined by microneutralization assay in vitro. mAb was pre-incubated with the lentiviral vectors prior to being added to HEK293 cells in a 96-well format. Percent inhibition is calculated relative to infection in the absence of mAb (FIG. 25 and Table 17). None of the tested antibodies showed neutralization activity against Zaire or Sudan GP-pseudotyped lentiviruses.

TABLE 17
50% Inhibitory titers

IC₅₀ (μg/ml)

| mAb | SUDV GP | EBOV GP |
|---|---|---|
| 16F6 | 0.04 | ND* |
| mAb114 | ND | 0.18 |
| 191L | >10 | >10 |
| 206L | >10 | >10 |
| 231L | >10 | >10 |
| 232L | >10 | >10 |
| 310L | >10 | >10 |
| 314L | ND | ND |
| 315L | >10 | ND |
| 396L | ND | ND |

*ND, not determined

### Example 6: Antibody Cocktail 545S+523S Protects Against SUDV Challenge when Administered Before or after Challenge and as a Single Dose

As described in Example 2, antibody cocktail 545S+523S was able to protect animals against a lethal 100 PFU challenge with SUDV Gulu. An additional experiment was performed to determine if this antibody cocktail could protect NHPs from challenge with SUDV Gulu at a higher dose (1000 PFU). Macaques (n=3) were challenged with 1000 PFU SUDV Gulu on day 0 (D0) and administered IV injections of a 1:1 cocktail of 545S+523S (50 mg/kg total) on D1, D2 and D3. A study control animal (n=1) was challenged with SUDV but did not receive treatment. The treated and control animals were compared to historical untreated controls (n=4). As shown in FIG. 30, all animals treated with the 545S+523S antibody cocktail survived challenge through the 28-day study period. In comparison, the study control and historical controls all succumbed to viral infection by day 8 post-challenge.

Further NHP studies using the 545S+523S antibody cocktail were performed to assess whether a single dose of the cocktail is sufficient for protection and whether the cocktail can protect against challenge when administered four or five days after challenge. Macaques were challenged with 1000 PFU of SUDV Gulu on DO and were subsequently infused with a 1:1 cocktail of 545S+523S (50 mg/kg total) on either D4 (n=3) or D5 (n=3). All treated animals survived challenged through the 28-day or 25-day study period (FIGS. 31A-31B). In comparison, in the D4 treatment study, 2 of 3 study controls and all historical controls (n=5) succumbed to infection (FIG. 31A). In the D5 treatment study, the study control (n=1) and 11 of 12 historical controls succumbed to infection (FIG. 31B).

Next, studies were performed to test lower doses of the 545S+523S antibody cocktail. Macaques were challenged with 1000 PFU SUDV Gulu on DO and intravenously infused with either 20 mg/kg (n=3) or 5 mg/kg (n=3) of a 1:1 cocktail of 545S+523S on D5. As shown in FIG. 32, all animals treated with 20 mg/kg of the antibody cocktail survived challenged through the 28-day study period, while 2 of 3 animals administered 5 mg/kg survived. In contrast, 12 of 13 historical control animals succumbed to infection. In this experiment, the study control animal survived challenge.

An additional study was performed to determine whether the 545S+523S antibody cocktail could protect NHPs from challenge if administered prior to challenge. Three days prior to challenge with 1000 PFU SUDV Gulu, macaques were infused with a 1:1 cocktail of 545S+523S (50 mg/kg total). All treated animals (n=3) survived challenge through the 25-day study period (FIG. 33). In contrast, the study control (n=1) and 11 of 12 historical controls succumbed to viral infection.

These studies demonstrate that antibody cocktail 545S+523S is capable of protecting NHPs from challenge with lethal doses of SUDV when administered either before or after challenge. This antibody cocktail was also shown to be highly effective at preventing death at doses as low as 20 mg/kg and 5 mg/kg.

### Example 7: Bispecific Antibodies Containing 545S and 523S Variable Domains in the CrossMab Format

In the CrossMab format, each arm of the bispecific antibody contains a different antibody variable fragment (Fv) domain. To maintain specificity of light and heavy chain associations in the Fv domains, the CrossMab format uses two features. The first feature is that one Fc domain contains so-called “knob” residues and the other Fc domain contains “hole” residues. The “knob” and “hole” residues prefer to associate with each other over self-association. This helps to promote hetero-associations between heavy chains. To promote proper light chain association, one of the arms of the antibody has the heavy chain CH1 and light chain CL domains swapped and is referred to as the “swapped” arm. Using the CrossMab format, two bispecific antibodies were designed using the combination of 545S and 523S variable Fv regions. BiSp201LS contains the Fv domain of antibody 523S (Fv523) in the “unswapped arm” and the Fv domain of 545S (Fv545) in the “swapped arm” (FIG. 26, left). In contrast, BiSp202LS contains Fv545 in the “unswapped arm” and Fv523 in the “swapped arm (FIG. 26, right). In order to provide half-life extending properties, the LS half-life extending mutations were included in the Fc domains of each of these molecules.

### Bispecific Antibody Sequences

In the sequences below, the leader peptide is in italics, the antibody variable region is underlined, and the CDRs are in bold. Knob and hole mutations and LS mutations are indicated by bold underline.

### BiSp201LS Sequences

Unswapped Fv523 heavy chain with Fe knob and LS
mutations
(SEQ ID NO: 152)
MGWSCIILFLVATATGVHSQVQLVQSGAELKKPGASVKVSCKASGYTFSD
HYVHWVRQAPGQGLEWMGRINPNSGGTNYAQRFLGRVTMTRDASISTAYL
DLSSLRSDDTAVYYCARSSSSWSGHYYYYMDVWGKGTTVTVSSASTKGPS
VFPLAPSSKSTSGGTAALGCLVKDYFPEPVTVSWNSGALTSGVHTFPAVL
QSSGLYSLSSVVTVSSSLGTQTYICNVNHKPSNTKVDKKVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPCRDELTKNQVSLWCLVKGFYPS
DIAVEWESNGQPENNYKTTPPVLDSDGSFFLYSKLTVDKSRWQQGNVFSC
SVLHEALHSHYTQKSLSLSPGK
Unswapped Fv523 lambda light chain
(SEQ ID NO: 153)
MGWSCIILFLVATATGVHSQSVLTQPPSVSAAPGQRVTISCSGSTSNIAS
NFVSWYQQLPGTAPKLLIYDNHKRPSGIPDRFSGSKSGTSATLGITGLQT
GDEADYYCGTWDSSLSAGVFGGGTKVTVLGQPKAAPSVTLFPPSSEELQA
NKATLVCLISDFYPGAVTVAWKADSSPVKAGVETTTPSKQSNNKYAASSY
LSLTPEQWKSHRSYSCQVTHEGSTVEKTVAPTECS
Fv545 swapped CH1CL heavy chain with Fc hole and
LS mutations
(SEQ ID NO: 154)
MGWSCIILFLVATATGVHSQVQLQESGPGLVKPSQTLSLTCTVSGGSISR
AGSYWSWIRQEPGKGLEWIGYISYSGSTYYNPSLESRVTMSLDTSKSQFS
LKLSSVTAADTAVYYCAREDRSGFYGLDVWGQGTTVTVSGQPKAAPSVTL
FPPSSEELQANKATLVCLSDFYPGAVTVAWKADSSPVKAGVETTTPSKQS
NNKYAASSYLSLTPEQWKSHRSYSCQVTHEGSTVEKTVAPTECSDKTHTC
PPCPAPELLGGPSVFLFPPKPKDTLMISRTPEVTCVVVDVSHEDPEVKFN
WYVDGVEVHNAKTXPREEQYNSTYRVVSVLTVLHQDWLNGKEYKCKVSNK
ALPAPIEKTISKAKGQPREPQVCTLPPSRDELTKNQVSLSCAVKGFYPSD
IAVEWESNGQPENNYKTTPPVLDSDGSFFLVSKLTVDKSRWQQGNVFSCS
VLHEALHSHYTQKSLSLSPGK
Fv545 swapped CH1CL lambda light chain
(SEQ ID NO: 155)
MGWSCIILFLVATATGVHSQSVLTQPSSLSASPGATASLTCTLRSGINVD
TYRIYWYQQKPGSPPQYLLRYKSDSDKHQGSGVPSRFSGSKDASANAGIL
LISGLQSADEADYYCMIWHSGAWVFGGGTKLTVQSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSC

### BiSp202LS Sequences

Unswapped Fv545 heavy chain with Fc knob and LS
mutations
(SEQ ID NO: 156)
MGWSCIILFLVATATGVHSQVQLQESGPGLVKPSQTLSLTCTVSGGSISR
AGSYWSWIRQEPGKGLEWIGYISYSGSTYYNPSLESRVTMSLDTSKSQFS
LKLSSVTAADTAVYYCAREDRSGFYGLDVWGQGTTVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPCRDELTKNQVSLWCLVKGFYPSDI
AVEWESNGQPENNYKTTPPVLDSDGSFFLYSKLTVDKSRWQQGNVFSCSV
LHEALHSHYTQKSLSLSPGK
Unswapped Fv545 lambda light chain
(SEQ ID NO: 157)
MGWSCIILFLVATATGVHSQSVLTQPSSLSASPGATASLTCTLRSGINVD
TYRIYWYQQKPGSPPQYLLRYKSDSDKHQGSGVPSRFSGSKDASANAGIL
LISGLQSADEADYYCMIWHSGAWVFGGGTKLTVQGQPKAAPSVTLFPPSS
EELQANKATLVCLISDFYPGAVTVAWKADSSPVKAGVETTTPSKQSNNKY
AASSYLSLTPEQWKSHRSYSCQVTHEGSTVEKTVAPTECS
Fv523 swapped CH1CL heavy chain with Fc hole and
LS mutations
(SEQ ID NO: 158)
MGWSCIILFLVATATGVHSQVQLVQSGAELKKPGASVKVSCKASGYTFSD
HYVHWVRQAPGQGLEWMGRINPNSGGTNYAQRFLGRVTMTRDASISTAYL
DLSSLRSDDTAVYYCARSSSSWSGHYYYYMDVWGKGTTVTVSGQPKAAPS
VTLFPPSSEELQANKATLVCLISDFYPGAVTVAWKADSSPVKAGVETTTP
SKQSNNKYAASSYLSLTPEQWKSHRSYSCQVTHEGSTVEKTVAPTECSDK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVCTLPPSRDELTKNQVSLSCAVKGF
YPSDIAVEWESNGQPENNYKTTPPVLDSDGSFFLVSKLTVDKSRWQQGNV
FSCSVLHEALESHYTQKSLSLSPG
Fv523 swapped CH1CL lambda light chain
(SEQ ID NO: 159)
MGWSCIILFLVATATGVHSQSVLTQPPSVSAAPGQRVTISCSGSTSNIAS
NFVSWYQQLPGTAPKLLIYDNHKRPSGIPDRFSGSKSGTSATLGITGLQT
GDEADYYCGTWDSSLSAGVFGGGTKVTVLSSASTKGPSVFPLAPSSKSTS
GGTAALGCLVKDYFPEPVTVSWNSGALTSGVHTFPAVLQSSGLYSLSSVV
TVPSSSLGTQTYICNVNHKPSNTKVDKKVEPKSC

### Results

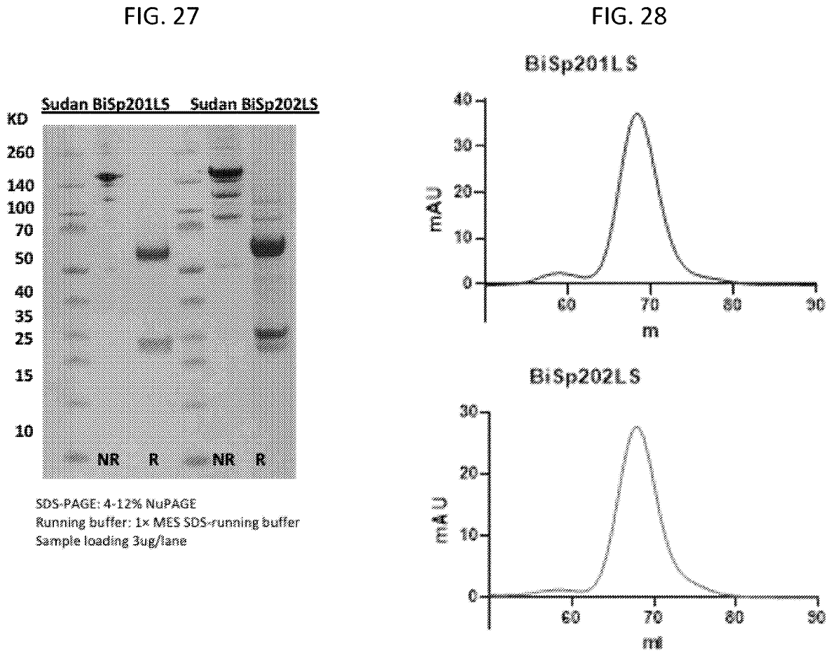

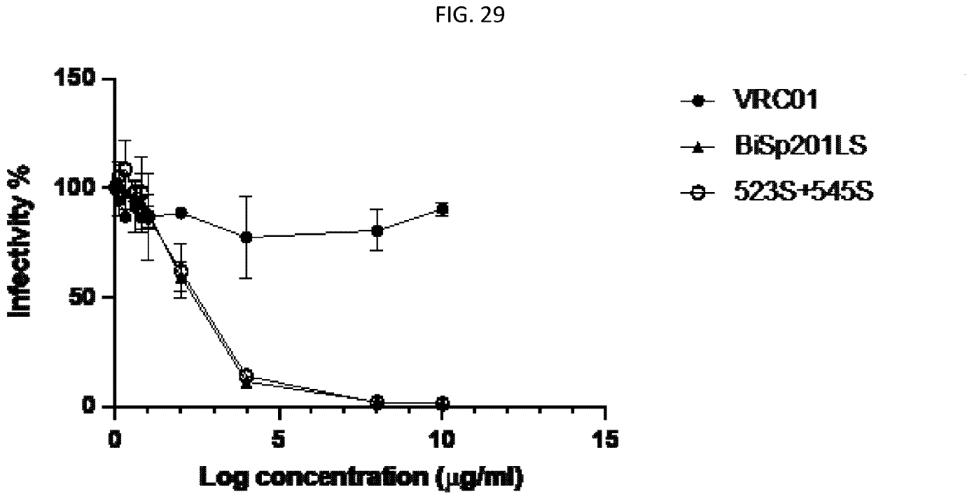

In order to make and test the bispecific antibody designs, 4 expression plasmids were generated to create BiSp201LS and another 4 expression plasmids were made to create BiSp202LS. Optimal expression and association of the heavy and light chains in CrossMab format antibodies can be dependent upon the relative amounts of plasmids transfected. A plasmid ratio of 1:1:1:1 was evaluated for BiSp201LS and BiSp202LS using Expi293 cells and Expifectamine transfection reagent. Using this ratio, the yield of BiSp201LS was 130 mg/liter and the yield of BiSp202LS was 38.2 mg/liter. Additionally, SDS-PAGE gel analysis, under reducing (R) and non-reducing (NR) conditions, and analytic HPLC were used to evaluate proper association and purity. The results showed that >95% of BiSp201LS showed a molecular weight consistent with properly associated heavy/light chains (FIG. 27 and FIG. 28). For BiSp202LS, the presence of some half-antibodies was noted, resulting in a lower amount of properly associated antibody (FIGS. 27 and 28). Nonetheless, >90% of the BiSp202LS molecules appeared to be properly associated.

The neutralizing capacity of BiSP201LS was tested using a pseudotyped lentivirus bearing the spike glycoprotein of SUDV. As expected, virus particles incubated with HIV-specific antibody VRC01 were not neutralized. However, BiSp201LS neutralized SUDV GP-pseudotyped lentivirus infection equivalent to an equal mixture of 523S and 545S (523S+545S) (FIG. 29).

To evaluate therapeutic efficacy of the bispecific antibodies for prophylactic or therapeutic indications, BiSP201LS and BiSP202LS are tested in the cynomolgus macaque model of SUDV infection described in the examples above.

### Example 8: BiSp201LS Protects Against SUDV Challenge when Administered Before or after Challenge

This example describes studies to determine whether the bispecific antibody BiSp201LS is capable of protecting NHPs against challenge with SUDV.

In a first study, macaques were challenged with 1000 PFU SUDV Gulu on DO and subsequently infused with BiSp201LS at a dose of 50 mg/kg on D4 (n=3) or D5 (n=5). All animals treated with the bispecific antibody on either D4 (FIG. 34A) or D5 (FIG. 34B) survived challenge through the study period (D28 or D25, respectively). In the D4 treatment study, the study control (n=1) and 11 of 12 historical controls succumbed to infection (FIG. 34A). In the D5 treatment study, 2 of 14 controls (2 study controls and 12 historical controls) succumbed to infection.

In a second study, macaques (n=3) were infused with the bispecific antibody BiSp201LS at a dose of 50 mg/kg three days prior to challenge with 1000 PFU of SUDV Gulu on DO. All treated animals survived challenge through the 25-day study period, whereas the study control (n=1) and 11 of 12 historical controls succumbed to infection (FIG. 35).

These results demonstrate that bispecific antibody BiSp201LS protects NHPs from lethal SUDV challenge when administered 4-5 days after challenge or when administered three days prior to challenge.

It will be apparent that the precise details of the methods or compositions described may be varied or modified without departing from the spirit of the described aspects of the disclosure. We claim all such modifications and variations that fall within the scope and spirit of the claims below.

1. A bispecific monoclonal antibody that specifically binds a Sudan virus (SUDV) or an Ebola virus (EBOV) glycoprotein, comprising:
a first antigen binding portion comprising a heavy chain variable (VH) domain and a light chin variable (VL) domain, wherein the VH domain comprises the heavy chain complementarity determining region (HCDR)1, HCDR2, and HCDR3 sequences of SEQ ID NO: 14 and the VL domain comprises the light chain complementarity determining region (LCDR)1, LCDR2, and LCDR3 sequences of SEQ ID NO: 16; and
a second antigen binding portion comprising a VH domain and a VL domain, wherein the VH domain comprises the HCDR1, HCDR2 and HCDR3 sequences of SEQ ID NO: 18 and the VL domain comprises the LCDR1, LCDR2 and LCDR3 sequences of SEQ ID NO: 20.

2. The bispecific monoclonal antibody of claim 1, wherein:
the HCDR1, HCDR2, and HCDR3 sequences of the first antigen binding portion are set forth as residues 26-35, 53-59, and 97-111 of SEQ ID NO: 14, and the LCDR1, LCDR2 and LCDR3 sequences of the first antigen portion are set forth as residues 26-34, 52-58, and 96-106 of SEQ ID NO: 16.

3. The bispecific monoclonal antibody of claim 1, wherein:
the HCDR1, HCDR2, and HCDR3 sequences of the second antigen binding portion are set forth as residues 26-33, 51-58, and 96-114 of SEQ ID NO: 18, and the LCDR1, LCDR2 and LCDR3 sequences of the second antigen binding portion are set forth as residues 26-33, 51-53, and 89-101 of SEQ ID NO: 20.

4. The bispecific monoclonal antibody of claim 1, wherein:
the VH domain of the first antigen binding portion comprises the amino acid sequence of SEQ ID NO: 14 and the VL domain of the first antigen binding portion comprises the amino acid sequence of SEQ ID NO: 16.

5. The bispecific monoclonal antibody of claim 1, wherein:
the VH domain of the second antigen binding portion comprises the amino acid sequence of SEQ ID NO: 18 and the VL domain of the second antigen binding portion comprises the amino acid sequence of SEQ ID NO: 20.

6. The bispecific monoclonal antibody of claim 1, wherein:
the first antigen binding portion comprises a heavy chain comprising SEQ ID NO: 152 or residues 20-473 of SEQ ID NO: 152, and a light chain comprising SEQ ID NO: 153 or residues 20-235 of SEQ ID NO: 153.

7. The bispecific monoclonal antibody of claim 1, wherein:
the second antigen binding portion comprises a heavy chain comprising SEQ ID NO: 154 or residues 20-472 of SEQ ID NO: 154, and a light chain comprising SEQ ID NO: 155 or residues 20-239 of SEQ ID NO: 155.

8. The bispecific monoclonal antibody of claim 1, wherein:
the first antigen binding portion comprises a heavy chain comprising SEQ ID NO: 156 or residues 20-470 of SEQ ID NO: 156, and a light chain comprising SEQ ID NO: 157 or residues 20-240 of SEQ ID NO: 157.

9. The bispecific monoclonal antibody of claim 1, wherein:
the second antigen binding portion comprises a heavy chain comprising SEQ ID NO: 158 or residues 20-474 of SEQ ID NO: 158, and a light chain comprising SEQ ID NO: 159 or residues 20-234 of SEQ ID NO: 159.

10. An isolated nucleic acid molecule encoding the bispecific monoclonal antibody of claim 1.

11. The isolated nucleic acid molecule of claim 10, operably linked to a promoter.

12. A vector comprising the nucleic acid molecule of claim 10.

13. A composition comprising a pharmaceutically acceptable carrier and the bispecific antibody of claim 1.

14. The composition of claim 13, wherein the composition is sterile and/or is in unit dosage form or a multiple thereof.

15. A method of detecting SUDV or EBOV glycoprotein in a sample, comprising contacting the sample with the bispecific monoclonal antibody of claim 1 under conditions sufficient to form an immune complex; and
detecting the presence of the immune complex in the sample, thereby detecting the SUDV or EBOV glycoprotein in the sample.

16. A method of detecting an Ebolavirus infection in a subject, comprising:
contacting a biological sample from the subject with bispecific monoclonal antibody of claim 1 under conditions sufficient to form an immune complex; and
detecting the presence of the immune complex in the sample, wherein the presence of the immune complex in the sample indicates that the subject has an Ebolavirus infection.

17. A method of inhibiting or treating an Ebolavirus infection in a subject, comprising administering to a subject with or at risk of an Ebolavirus infection a therapeutically effective amount of the bispecific monoclonal antibody of claim 1, thereby inhibiting or treating the Ebolavirus infection.

18. The method of claim 17, further comprising administering to the subject one or more additional therapies for treating the Ebolavirus infection.

19. The method of claim 17, wherein the subject has been exposed to an Ebolavirus but has not been diagnosed as having an Ebolavirus infection.

20. The method of claim 19, wherein the bispecific monoclonal antibody is administered no more than 2, no more than 3, no more than 4, no more than 5, no more than 6, no more than 7, no more than 8, no more than 9, no more than 10, no more than 15 or no more than 20 days following exposure to the Ebolavirus or infection with the Ebolavirus.

21. The method of claim 17, wherein the subject is at risk of an Ebolavirus infection but has not yet been exposed to an Ebolavirus.

22. The method of claim 21, wherein the bispecific monoclonal antibody is administered about 16 weeks, about 14 weeks, about 12 weeks, about 10 weeks, about 8 weeks, about 6 weeks, about 4 weeks, about 2 weeks, about 1 one week, about 6 days, about 5 days, about 4 days, about 3 days, about 2 days and/or about 1 day prior to exposure to the Ebolavirus.

23. The method of claim 17, wherein the bispecific monoclonal antibody is administered in multiple doses.

24. The method of claim 23, wherein the bispecific monoclonal antibody is administered in two, three, four or five doses.

25. The method of claim 17, wherein the bispecific monoclonal antibody is administered in a single dose.

26. The method of claim 17, wherein the Ebolavirus is SUDV or EBOV.

27. (canceled)

28. A monoclonal antibody or antigen binding fragment thereof that specifically binds a Sudan virus (SUDV) or an Ebola virus (EBOV) glycoprotein, comprising a heavy chain variable domain (VH) and a light chin variable domain (VL) comprising the heavy chain complementarity determining region (HCDR)1, HCDR2, and HCDR3 sequences, and the light chain complementarity determining region (LCDR)1, LCDR2, and LCDR3 sequences, of the VH and VL set forth as any one of:
SEQ ID NOs: 14 and 16, respectively;
SEQ ID NOs: 18 and 20, respectively;
SEQ ID NOs: 2 and 4, respectively;
SEQ ID NOs: 6 and 8, respectively;
SEQ ID NOs: 10 and 12, respectively;
SEQ ID NOs: 22 and 24, respectively;
SEQ ID NOs: 26 and 28, respectively;
SEQ ID NOs: 30 and 32, respectively;
SEQ ID NOs: 34 and 36, respectively;
SEQ ID NOs: 38 and 40, respectively;
SEQ ID NOs: 42 and 44, respectively;
SEQ ID NOs: 46 and 48, respectively;
SEQ ID NOs: 50 and 52, respectively;
SEQ ID NOs: 54 and 56, respectively;
SEQ ID NOs: 58 and 60, respectively;
SEQ ID NOs: 62 and 64, respectively;
SEQ ID NOs: 66 and 68, respectively;
SEQ ID NOs: 70 and 72, respectively;
SEQ ID NOs: 74 and 76, respectively;
SEQ ID NOs: 78 and 80, respectively;
SEQ ID NOs: 82 and 84, respectively;
SEQ ID NOs: 86 and 88, respectively;
SEQ ID NOs: 90 and 92, respectively;
SEQ ID NOs: 94 and 96, respectively;
SEQ ID NOs: 98 and 100, respectively;
SEQ ID NOs: 102 and 104, respectively;
SEQ ID NOs: 106 and 108, respectively;
SEQ ID NOs: 110 and 112, respectively;
SEQ ID NOs: 114 and 116, respectively;
SEQ ID NOs: 118 and 120, respectively;
SEQ ID NOs: 122 and 124, respectively;
SEQ ID NOs: 126 and 128, respectively;
SEQ ID NOs: 130 and 132, respectively; or
SEQ ID NOs: 134 and 136, respectively.


SEQUENCE LISTING (159 sequences)

SEQ ID NO: 1 - DNA, 366 bp, synthetic construct
  source 1..366: mol_type = other DNA
gaagagagattggtggagtctgggggaggcctggtccagcctggggggtccctgagactctcgtgtgcagcctctggattcacttttggcgattatgccatgcactgggtccgccaagttcctgggaagagtctcgagtggctctctggtataagttggagtggttatacgacatactctgcagactccgtcaagggccgattcaccatctccagagacaacgccaagaattccctgtatctgcaaatgaaccgcctgagacctgaggacacaggcctctattactgtgtgaccttgataggggcgattggaattaaagactcattggatgtctggggccggggagttctggtcaccgtctcctca

SEQ ID NO: 2 - protein, 122 aa, synthetic construct
EERLVESGGGLVQPGGSLRLSCAASGFTFGDYAMHWVRQVPGKSLEWLSGISWSGYTTYSADSVKGRFTISRDNAKNSLYLQMNRLRPEDTGLYYCVTLIGAIGIKDSLDVWGRGVLVTVSS

SEQ ID NO: 3 - DNA, 333 bp, synthetic construct
  source 1..333: mol_type = other DNA
cagtctgtgctgacgcagccgccctcagtgtctggggcgcccgggcagagggtcaccgtctcctgctctgggagcagctccaacattggggcgggtaattatgtacagtggtaccagcaacttccaggaacggcccccaaagtcctcatctatcaaactgagaagcgaccctcaggcacttctgatcgattctctggctccaagtctgatacctcggcctccctgaccatcaatggactccagtctgaggatgaggctgattattactgccaggtctatgacagcaatttgaatggttgggtattcggaggagggacccggctgacagtacta

SEQ ID NO: 4 - protein, 111 aa, synthetic construct
QSVLTQPPSVSGAPGQRVTVSCSGSSSNIGAGNYVQWYQQLPGTAPKVLIYQTEKRPSGTSDRFSGSKSDTSASLTINGLQSEDEADYYCQVYDSNLNGWVFGGGTRLTVL

SEQ ID NO: 5 - DNA, 366 bp, synthetic construct
  source 1..366: mol_type = other DNA
gaagtgcagttggtggagtctgggggaggcctggtccagcctggggggtccctgacactctcgtgtgcagcctctggattcacctttggtgattatgtcatgcactgggtccgccaagttccagggaggggtctagagtggctctctgctgtaagttggagtggctacaccacatactctgcagactccgtcaagggccgattcaccatctccagagacaacgccaagaactccctctatctacaaatgagccgcctgggaactgcggacacaggcctctattactgtgtaaccctgataggggcgattggaatacgcgactccttcgatgcctggggccggggagttctggtcaccgtctcctca

SEQ ID NO: 6 - protein, 122 aa, synthetic construct
EVQLVESGGGLVQPGGSLTLSCAASGFTFGDYVMHWVRQVPGRGLEWLSAVSWSGYTTYSADSVKGRFTISRDNAKNSLYLQMSRLGTADTGLYYCVTLIGAIGIRDSFDAWGRGVLVTVSS

SEQ ID NO: 7 - DNA, 333 bp, synthetic construct
  source 1..333: mol_type = other DNA
cagtctgtgctgacgcagccgccctcaatgtctggggcgcccgggcagagggtcaccgtctcctgcacggggagcagctccaacattggagcgggtaattatgtgcagtggtaccagcaacttccaggaacggcccccaaagtcctcatctatcaaactgataagcgaccctcaggtatttctgatcgattctctggctccaagtctggtacctcagcctccctgaccatcagtggactccagtctgaggatgaggctgattattactgccaggtctatgacagcagtttgaatggttgggtattcggaggagggacccggctgacagtacta

SEQ ID NO: 8 - protein, 111 aa, synthetic construct
QSVLTQPPSMSGAPGQRVTVSCTGSSSNIGAGNYVQWYQQLPGTAPKVLIYQTDKRPSGISDRFSGSKSGTSASLTISGLQSEDEADYYCQVYDSSLNGWVFGGGTRLTVL

SEQ ID NO: 9 - DNA, 354 bp, synthetic construct
  source 1..354: mol_type = other DNA
caggtgcagctgcaggagtcggggccaggactggtgaagccttcacagaccctgtccctcacctgcactgtctctggtggctccttcagtagcggtttctactggagttggatccgccagcgcccagggaagggcctggagtgggtcgggtacacctattacagtgggagcacctactacaacccgtccctccagagtcgagtcaccatatcagtggacacgtctaagaaccagttctccctgaacctgagctctgtgactgccgcggacacggccgtgtatcactgtgcgagagccgactacggtggcctctttgactactggggccagggaattctggtcaccgtctcctca

SEQ ID NO: 10 - protein, 118 aa, synthetic construct
QVQLQESGPGLVKPSQTLSLTCTVSGGSFSSGFYWSWIRQRPGKGLEWVGYTYYSGSTYYNPSLQSRVTISVDTSKNQFSLNLSSVTAADTAVYHCARADYGGLFDYWGQGILVTVSS

SEQ ID NO: 11 - DNA, 345 bp, synthetic construct
  source 1..345: mol_type = other DNA
cagtctgtgctgactcagccgtcttccctctctgcatctcctggagcatcagccagtctcacctgcaccttgcgcagtggcatcaatgttggtacttacaggatattctggtaccagcagaagccagggagtcctccccagtttctcctgaggtacaactcagactcagataagcagcagggctctggagtccccagccgcttctctggatccaaagatgcttcggccaatgcagggattttactcatctctgggctccagtctgaggatgaggctgactattactgtatgatttggcacaccagcgcttgggtgttcggcggagggaccaagctgaccgtccta

SEQ ID NO: 12 - protein, 115 aa, synthetic construct
QSVLTQPSSLSASPGASASLTCTLRSGINVGTYRIFWYQQKPGSPPQFLLRYNSDSDKQQGSGVPSRFSGSKDASANAGILLISGLQSEDEADYYCMIWHTSAWVFGGGTKLTVL

SEQ ID NO: 13 - DNA, 363 bp, synthetic construct
  source 1..363: mol_type = other DNA
caggtgcagctgcaggagtcggggccaggactggtgaagccttcacagaccctgtccctcacctgcactgtctctggtggctccatcagccgtgctggttcctactggagctggatccgccagcacccagggaagggcctggagtggattggctacatctcttacagtgggagcacctactacaacccgtccctcgagagtcgagttaccatgtcactagacacgtctaagagccagttctcccttaagttgagctctgtgactgccgcggacacggccgtctattattgtgcgagagaggaccgctcgggcttctacggtttggacgtctggggccaagggaccacggtcaccgtctcctca

SEQ ID NO: 14 - protein, 121 aa, synthetic construct
QVQLQESGPGLVKPSQTLSLTCTVSGGSISRAGSYWSWIRQHPGKGLEWIGYISYSGSTYYNPSLESRVTMSLDTSKSQFSLKLSSVTAADTAVYYCAREDRSGFYGLDVWGQGTTVTVSS

SEQ ID NO: 15 - DNA, 345 bp, synthetic construct
  source 1..345: mol_type = other DNA
cagtctgtgctgactcagccgtcttccctctctgcatctcctggagcaacagccagtctcacttgcaccttgcgcagtggcatcaatgttgatacctacaggatatactggtaccagcagaagccagggagtcctccccagtatctcctgaggtacaaatcagactcggataaacatcagggctctggagtccccagccgcttctctggatccaaagatgcttcggccaatgcagggattttactcatctctgggctccagtctgcggatgaggctgactattactgtatgatatggcacagcggcgcttgggtgttcggcggagggaccaagctgaccgtccaa

SEQ ID NO: 16 - protein, 115 aa, synthetic construct
QSVLTQPSSLSASPGATASLTCTLRSGINVDTYRIYWYQQKPGSPPQYLLRYKSDSDKHQGSGVPSRFSGSKDASANAGILLISGLQSADEADYYCMIWHSGAWVFGGGTKLTVQ

SEQ ID NO: 17 - DNA, 372 bp, synthetic construct
  source 1..372: mol_type = other DNA
caggtgcagctggtgcagtctggggctgagttgaagaagcctggggcctcagtgaaggtctcctgcaaggcttctggatacaccttcagcgaccactatgtgcactgggtgcgacaggcccctggacaagggcttgagtggatgggacggatcaaccctaacagtggtggcacaaactatgcacagaggtttctgggcagggtcaccatgaccagggacgcgtccatcagcacagcctacctggacctgagcagtctgagatctgacgacacggccgtctattattgtgcgagaagtagcagcagttggtcggggcactactactactacatggacgtctggggcaaagggaccacggtcaccgtctcgtca

SEQ ID NO: 18 - protein, 124 aa, synthetic construct
QVQLVQSGAELKKPGASVKVSCKASGYTFSDHYVHWVRQAPGQGLEWMGRINPNSGGTNYAQRFLGRVTMTRDASISTAYLDLSSLRSDDTAVYYCARSSSSWSGHYYYYMDVWGKGTTVTVSS

SEQ ID NO: 19 - DNA, 330 bp, synthetic construct
  source 1..330: mol_type = other DNA
cagtctgtgctgacgcagccgccctcagtgtctgcggccccaggacagagggtcaccatctcctgctctggaagcacctccaacattgcgagtaattttgtatcctggtaccagcagctcccaggaacagcccccaaactcctcatttatgataatcataagcgaccctcagggattcctgaccgattctctggctccaagtctggcacgtcagccaccctgggcatcaccggactccagactggggacgaggccgattattactgcggaacatgggatagcagcctgagtgctggggtcttcggcggagggaccaaggtgaccgtccta

SEQ ID NO: 20 - protein, 110 aa, synthetic construct
QSVLTQPPSVSAAPGQRVTISCSGSTSNIASNFVSWYQQLPGTAPKLLIYDNHKRPSGIPDRFSGSKSGTSATLGITGLQTGDEADYYCGTWDSSLSAGVFGGGTKVTVL

SEQ ID NO: 21 - DNA, 378 bp, synthetic construct
  source 1..378: mol_type = other DNA
gaagtgcagttggtggagtctgggggaggcctggtcaagccgggggggtccctgagactctcctgtgcagcctctggattcaccttcagtagttataccatgaactgggtccgccaggctccagggaaggggctggagtgggtctcatgcattaggagtagcagtagtgacaaatactacgcagactcagtgaagggccgattcaccatctccagagacaacaccaagaactcactgtatctgcaaatgaacagcctgagagccgacgacacggctgtgtattactgtgcgagagatctaggcggtgctaataaccgctactacttcgactatggtatggacgtctggggccaagggaccacggtcaccgtctcctca

SEQ ID NO: 22 - protein, 126 aa, synthetic construct
EVQLVESGGGLVKPGGSLRLSCAASGFTFSSYTMNWVRQAPGKGLEWVSCIRSSSSDKYYADSVKGRFTISRDNTKNSLYLQMNSLRADDTAVYYCARDLGGANNRYYFDYGMDVWGQGTTVTVSS

SEQ ID NO: 23 - DNA, 324 bp, synthetic construct
  source 1..324: mol_type = other DNA
gaaatagtgatgacgcagtctccagccaccctgtctgtgtctccagggggaagagccaccctctcctgcagggccagtcagagtgtttacaccaacttagcctggtaccagcagaaacctggccaggctcccaggctcctcatctatgatgcatccaccagggccactggtatcccagccaggttcagtggcagtgggtctgggacagagttcactctcaccatcagcagcctgcagtgtgaggattttgcagtttattactgtcagcactataataactggcccctgtacacttttggccaggggaccaagctggagatcaaa

SEQ ID NO: 24 - protein, 108 aa, synthetic construct
EIVMTQSPATLSVSPGGRATLSCRASQSVYTNLAWYQQKPGQAPRLLIYDASTRATGIPARFSGSGSGTEFTLTISSLQCEDFAVYYCQHYNNWPLYTFGQGTKLEIK

SEQ ID NO: 25 - DNA, 366 bp, synthetic construct
  source 1..366: mol_type = other DNA
caggtgcagctggtgcagtctggagctgaagtgaagaagcctggggcctcagtgaaggtctcctgcaaggcttctggttacacctttaccaactatgccatcacctgggtgcgacaggcccctggacaagggcttgagtggatgggatggatcagcgctttcaatggtcaaataaactatgcacagaagttccagggcagagtcgccatgaccacagacacatccacgagcacagcctacatggagctgaggagcctgagatctgacgacacggccgtctattactgtgcgagagccccccctatgtggttctctggggccaaatttgactactggggccagggaaccctggtcaccgtctcctca

SEQ ID NO: 26 - protein, 122 aa, synthetic construct
QVQLVQSGAEVKKPGASVKVSCKASGYTFTNYAITWVRQAPGQGLEWMGWISAFNGQINYAQKFQGRVAMTTDTSTSTAYMELRSLRSDDTAVYYCARAPPMWFSGAKFDYWGQGTLVTVSS

SEQ ID NO: 27 - DNA, 327 bp, synthetic construct
  source 1..327: mol_type = other DNA
cagtctgtgctgactcagccaccctcagtgtctgggacccccgggcagagggtcaccatctcttgttctggaagcagctccaacatcggaagcaatactgtaaactggtaccagcagctcccaggaacggcccccaaactcctcatctatagtaatgatcagcggccctcaggggtccctgaccgattctctggctccaagtctggcacctctgcctccctggccatcagtgggctccggtccgaggatgaggctgattattactgtgcagcatgggatgaaaccctgaggagggtgttcggcggagggaccaggctgaccgtccta

SEQ ID NO: 28 - protein, 109 aa, synthetic construct
QSVLTQPPSVSGTPGQRVTISCSGSSSNIGSNTVNWYQQLPGTAPKLLIYSNDQRPSGVPDRFSGSKSGTSASLAISGLRSEDEADYYCAAWDETLRRVFGGGTRLTVL

SEQ ID NO: 29 - DNA, 375 bp, synthetic construct
  source 1..375: mol_type = other DNA
caggtgcagctgcaggagtcggggccaggactggtgaagccttcacagaccctgtccctcacctgcactgtctctggtggctccatcagcagtggtggttaccactggagctggatccgccagcacccagggaagggcctggagtggattgggaacatctattacaatgggcgcacctactataatccgtccctcaagagtcgagttaccatatcagtagacacgtctgagaacgaattctccctgaagttgagctctgtgactgccgcggacacggccgtgtattactgcgcgagagatcggaggaggcctagaatcgggcacgactacggtatggacgtctggggccaagggaccaaggtcatcgtctcctca

SEQ ID NO: 30 - protein, 125 aa, synthetic construct
QVQLQESGPGLVKPSQTLSLTCTVSGGSISSGGYHWSWIRQHPGKGLEWIGNIYYNGRTYYNPSLKSRVTISVDTSENEFSLKLSSVTAADTAVYYCARDRRRPRIGHDYGMDVWGQGTKVIVSS

SEQ ID NO: 31 - DNA, 324 bp, synthetic construct
  source 1..324: mol_type = other DNA
gacatccagatgacccagtctccatcctccctgtctgcatctgtaggagacagagtcaccatctcttgccgggcaagtcagagcattagcaactttttaaattggtatcagcagaaaccagggcaagcccctaaactcctgatctacactgcatccaatttgcaaagtggggtcccatcaaggttcagtggcagtggatctgggacagatttcactctcaccatcagcagtctgcaacctgaagattttgcaacttactactgtcaacagatttacaataccggcatgtactcttttggccaggggaccaagctggagatcaaa

SEQ ID NO: 32 - protein, 108 aa, synthetic construct
DIQMTQSPSSLSASVGDRVTISCRASQSISNFLNWYQQKPGQAPKLLIYTASNLQSGVPSRFSGSGSGTDFTLTISSLQPEDFATYYCQQIYNTGMYSFGQGTKLEIK

SEQ ID NO: 33 - DNA, 375 bp, synthetic construct
  source 1..375: mol_type = other DNA
caggtgcagctggtacaatctgggtctgagttgaagaagcctggggcctcagtgaaggtttcctgcaaggcttctggatacaccttcacaagctatactgtgaattgggtgcgacaggcccctggacaagggcttgagtggatgggatggatcaacaccaacactgggaacccaacgtatgcccagggcttcacaggacggtttgtcttctcctcggacacctctgtcagcacggcatatctgcagatcagcgacctaaaggctgaggacacagccatgtattactgtgcgagacacgtcgcatacgatctttggagtggtcacacggttacctttgaccactggggccagggaaccctggtcacagtctcctca

SEQ ID NO: 34 - protein, 125 aa, synthetic construct
QVQLVQSGSELKKPGASVKVSCKASGYTFTSYTVNWVRQAPGQGLEWMGWINTNTGNPTYAQGFTGRFVFSSDTSVSTAYLQISDLKAEDTAMYYCARHVAYDLWSGHTVTFDHWGQGTLVTVSS

SEQ ID NO: 35 - DNA, 330 bp, synthetic construct
  source 1..330: mol_type = other DNA
cagtctgccctgactcagcctccctccgcgtccgggtctcctggacagtcagtcaccatctcctgcactggaaccagcagtgacgttggtgcttataactacgtctcctggtaccagcagcacccaggcaaagcccccaaactcatcatttatgaggtcactaagcggccctcaggggtccctgatcgcttctctggctccaagtctggcaacacggcctccctgaccgtctctgggctccaggctgaggatgaggctgattattactgcagctcaaaagcaggcggaaaaacttatgtcttcggaactgggaccaaggtcaccgtccta

SEQ ID NO: 36 - protein, 110 aa, synthetic construct
QSALTQPPSASGSPGQSVTISCTGTSSDVGAYNYVSWYQQHPGKAPKLIIYEVTKRPSGVPDRFSGSKSGNTASLTVSGLQAEDEADYYCSSKAGGKTYVFGTGTKVTVL

SEQ ID NO: 37 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacagcttcgccagttactatatacactgggtgcgacaggcccctggacaagggcttgagtggatgggaataatcaaccctagtggtggtaccacaagctacgcacagaagttccagggcagagtcaccatgaccagggacacgtccacgagcacagtctacatggagctgagcagcctgggatctgaggacacggccgtgtattactgtgcgagaatagtcgatcgagttacgatatatctgaatggggttgataactactggggccagggaaccctggtcaccgtctcctca

SEQ ID NO: 38 - protein, 124 aa, synthetic construct
QVQLVQSGAEVKKPGASVKVSCKASGYSFASYYIHWVRQAPGQGLEWMGIINPSGGTTSYAQKFQGRVTMTRDTSTSTVYMELSSLGSEDTAVYYCARIVDRVTIYLNGVDNYWGQGTLVTVSS

SEQ ID NO: 39 - DNA, 333 bp, synthetic construct
  source 1..333: mol_type = other DNA
gaaatagtgatgacgcagtctccagccaccctgtctgtgtctccaggggaaagagtcaccctctcctgcagggccagtcagagcgttagcagcaacttagcctggttccagcagacacctggccaggctcccaggctcctcatctatggtgcatccaccagggcctctggtgtcccagccaggttcagtggcagtgggtctgggacagaattcactctcaccatcagcagcctgcagtctgaagattttgcagtctattactgtctgcagtataataattggcctccgggttggaagtacggttttggccaggggaccaagctggagatcaaa

SEQ ID NO: 40 - protein, 111 aa, synthetic construct
EIVMTQSPATLSVSPGERVTLSCRASQSVSSNLAWFQQTPGQAPRLLIYGASTRASGVPARFSGSGSGTEFTLTISSLQSEDFAVYYCLQYNNWPPGWKYGFGQGTKLEIK

SEQ ID NO: 41 - DNA, 372 bp, synthetic construct
  source 1..372: mol_type = other DNA
gaagtgcagctggtggagtctgggggaggcttgggacagcctggggggtccctgagactctcctgtgcagcctctggattcaagttcaaaatctttggcatgaactgggtccgccaggctccagggaaggggctggagtgggtttcatacattagtagtagaagtagtatcatatattatgcagactctgtgaagggccggttcaccatctccagagacgatgccacgaattcactgtttctgcaaatgaacagcctgagagacgaggacacggctatgtattactgtgtgagagattacaatttgattgacttcgtttgggggagtctatttgactactggggccagggaatcctggtcaccgtctcctca

SEQ ID NO: 42 - protein, 124 aa, synthetic construct
EVQLVESGGGLGQPGGSLRLSCAASGFKFKIFGMNWVRQAPGKGLEWVSYISSRSSIIYYADSVKGRFTISRDDATNSLFLQMNSLRDEDTAMYYCVRDYNLIDFVWGSLFDYWGQGILVTVSS

SEQ ID NO: 43 - DNA, 321 bp, synthetic construct
  source 1..321: mol_type = other DNA
gccatccggatgacccagtctccctcctcactctcagcatctacaggagacagcgtcaccatcacttgtcgggcgagtcagaatattgccagttatttagcctggtatcagcaaataccaggaaaagcccctaagctcctgatttatgttgcatccactttgcaaagtggggtcccatccaggttcagcggcagtggatctgggacagatttcactctcaccatcagctccctgcagtctgaagactttgcaacttattactgtcagcagtattatcgttaccctccgacgttcggccaggggaccaaggtggaaatcaaa

SEQ ID NO: 44 - protein, 107 aa, synthetic construct
AIRMTQSPSSLSASTGDSVTITCRASQNIASYLAWYQQIPGKAPKLLIYVASTLQSGVPSRFSGSGSGTDFTLTISSLQSEDFATYYCQQYYRYPPTFGQGTKVEIK

SEQ ID NO: 45 - DNA, 378 bp, synthetic construct
  source 1..378: mol_type = other DNA
caggtgcagctggtgcagtctggggctgaggtgaagaagcctggggcctcagtgagggtttcctgcaaggcatctggatacaccttcaccagctactatatgcactgggtccgacaggcccctggacaagggcttgagtggatgggacagatcaaccctagtggcggcaccacaaggtacgaacggaagttccagggcagaatcaccgtgacccgggacacgtccacgagcacagtctacttggaactgagcagcctgagatctgaggacacggccagttatttttgtgcgtgtcttcgggataaggtgacgtattacgatctctggagtggagtggactccgtttggggccagggaaccctggtcaccgtctcctca

SEQ ID NO: 46 - protein, 126 aa, synthetic construct
QVQLVQSGAEVKKPGASVRVSCKASGYTFTSYYMHWVRQAPGQGLEWMGQINPSGGTTRYERKFQGRITVTRDTSTSTVYLELSSLRSEDTASYFCACLRDKVTYYDLWSGVDSVWGQGTLVTVSS

SEQ ID NO: 47 - DNA, 321 bp, synthetic construct
  source 1..321: mol_type = other DNA
gacatccagatgacccagtctccatcctcactgtctgcatctgtaggagacagagtcaccatcacttgtcgggcgagtcaggatattagcacctggttagcctggtatcagcagaaaccagagaaagcccctaagtccctgatctatgctgcatccactttggaaactggggtcccatccaggttcagcggcagtggatctgggacagatttcactctcaccatcagcagcctgcagcctgaagattttgcaacttattactgcgagcagtataatggtttccctcggacttttggccaggggaccaagctggacatcaaa

SEQ ID NO: 48 - protein, 107 aa, synthetic construct
DIQMTQSPSSLSASVGDRVTITCRASQDISTWLAWYQQKPEKAPKSLIYAASTLETGVPSRFSGSGSGTDFTLTISSLQPEDFATYYCEQYNGFPRTFGQGTKLDIK

SEQ ID NO: 49 - DNA, 372 bp, synthetic construct
  source 1..372: mol_type = other DNA
caggtccagctggtgcagtctggggctgaggtgaagaagcctgggtcctcggtgaaggtctcctgcaaggcttctggaggcaccttcagcagttatgctatcagctgggtgcgacaggcccctggacaagggcttgagtggttgggaaggatcctccctgcccttggtttagcaaactacgcacagaagtaccaggacagagtcaagattaccgcggacaaatccacgagcacagcctacatggagctgagcagactgggatctgaggacacggccgtgtattactgtgcgagagatcgctgggtgggagctgcccggcgggggcagtactttgactactggggccagggaacccaggtcaccgtctcctca

SEQ ID NO: 50 - protein, 124 aa, synthetic construct
QVQLVQSGAEVKKPGSSVKVSCKASGGTFSSYAISWVRQAPGQGLEWLGRILPALGLANYAQKYQDRVKITADKSTSTAYMELSRLGSEDTAVYYCARDRWVGAARRGQYFDYWGQGTQVTVSS

SEQ ID NO: 51 - DNA, 321 bp, synthetic construct
  source 1..321: mol_type = other DNA
gacatccagatgacccagtctcctgccaccctgtctgcatctgtaggagacagagtcaccatcacttgccgggccagtcagagtattggtacttggttggcctggtatcagcagaaaccagggaaagcccctaaactgctgatctatgatgcctccagtttggaaagtggggtcccaccaaggttcagcggcagtgggtctgaaacagaattcactctcaccatcagcagcctgcagcctgatgattttgcaacttactactgccaccagtataagagttattcgtacacttttggccaggggaccaagctggagatcaaa

SEQ ID NO: 52 - protein, 107 aa, synthetic construct
DIQMTQSPATLSASVGDRVTITCRASQSIGTWLAWYQQKPGKAPKLLIYDASSLESGVPPRFSGSGSETEFTLTISSLQPDDFATYYCHQYKSYSYTFGQGTKLEIK

SEQ ID NO: 53 - DNA, 363 bp, synthetic construct
  source 1..363: mol_type = other DNA
gaagtgcagctggtggagtctgggggaggcgtggtccagcctgggaggtccctgagactctcctgtgcagcctctggattcaccttcaaaacctatactatgcactgggtccgccaggctccaggcaagggactagagtgggtggcagttataacagatgatggagttaataaaaagtacgcagactccgtgaagggccgattcaccatctccagagacaattccaagaacacgctgtatctgcaaatgaacagcctgagacttgaggacacggctgtgtattactgtgcgagaagtgttgtggttctggctgcttaccctcttgactactggggccagggaaccatggtcaccgtctcctca

SEQ ID NO: 54 - protein, 121 aa, synthetic construct
EVQLVESGGGVVQPGRSLRLSCAASGFTFKTYTMHWVRQAPGKGLEWVAVITDDGVNKKYADSVKGRFTISRDNSKNTLYLQMNSLRLEDTAVYYCARSVVVLAAYPLDYWGQGTMVTVSS

SEQ ID NO: 55 - DNA, 321 bp, synthetic construct
  source 1..321: mol_type = other DNA
gacatccagatgacccagtctccatcctccctgtctgcatctgtaggagacagagtcaccatcacttgccgggcaagtcagaccattagcacctatttacattggtatcagcagagaccagggaaagcccctaagttgttgatctatgctgcatccagtttgcaaagtggggtcccatcaagggtcagtggcagtggatctgggacagatttcactctcaccatcaccagtctgcaacctgaagattttgcaacttactactgtctacagacttacagtcttcctctcactttcggcggagggaccaacgtggagatcaaa

SEQ ID NO: 56 - protein, 107 aa, synthetic construct
DIQMTQSPSSLSASVGDRVTITCRASQTISTYLHWYQQRPGKAPKLLIYAASSLQSGVPSRVSGSGSGTDFTLTITSLQPEDFATYYCLQTYSLPLTFGGGTNVEIK

SEQ ID NO: 57 - DNA, 360 bp, synthetic construct
  source 1..360: mol_type = other DNA
caggtccagctggtgcagtctggggctgaggtgaagaagccggggtcctcggtgaaagtctcctgcaaggcttctggaggcaccttcagcaactatgctatcaactgggtgcgacaggcccctggacaagggcttgagtggatgggaaggatcatccctttccttggtttagcaaactacgcacagaagctccagggcagagtcacgattaccgcggacaaatccacgagcacagcctacatggagctgagcagcctgagatctgaggacacggccctttattactgtgcgacaccccctggatccaggtatagtggctttgactcctggggccagggaaccctggtcaccgtctcctca

SEQ ID NO: 58 - protein, 120 aa, synthetic construct
QVQLVQSGAEVKKPGSSVKVSCKASGGTFSNYAINWVRQAPGQGLEWMGRIIPFLGLANYAQKLQGRVTITADKSTSTAYMELSSLRSEDTALYYCATPPGSRYSGFDSWGQGTLVTVSS

SEQ ID NO: 59 - DNA, 318 bp, synthetic construct
  source 1..318: mol_type = other DNA
gaaattgtgttgacgcagtctccaggcaccctgtctttgtctccaggggaaagagccaccctctcctgcagggccagtcagagttttaacaactacttagcctggtaccagcagaaacctggccaggctcccaggctcctcatcaatggtgcatccagcagggccactggcatcccagacaggttcagtggcagtgggtctgggacagacttcactctcaccatcagcagactggagcctgaagattttgcagtgtattactgtcagcagtatggtagctcacctactttcggccctgggaccagagtggaaatcaaa

SEQ ID NO: 60 - protein, 106 aa, synthetic construct
EIVLTQSPGTLSLSPGERATLSCRASQSFNNYLAWYQQKPGQAPRLLINGASSRATGIPDRFSGSGSGTDFTLTISRLEPEDFAVYYCQQYGSSPTFGPGTRVEIK

SEQ ID NO: 61 - DNA, 360 bp, synthetic construct
  source 1..360: mol_type = other DNA
caggtgcagctggtgcagtctggggctgaggtgaagaagcctgggtcctcggtgaaggtctcctgcaaggcctctggaggcaccttcagcaactatgctatcaactgggtgcgacaggcccctggacaagggcttgagtggatgggaaggaccatccccttccttggtttaacaaactacgcagagaacttccaggccagagtcacgattaccgcggacaagtccacgggcacgtcatacatgcagctgagcagcctcagatctgaggacacggccgtgtatttttgtgcgaaagctgccaccagtggctggtccccctttgacaactggggccagggaaccctggtcaccgtctcctca

SEQ ID NO: 62 - protein, 120 aa, synthetic construct
QVQLVQSGAEVKKPGSSVKVSCKASGGTFSNYAINWVRQAPGQGLEWMGRTIPFLGLTNYAENFQARVTITADKSTGTSYMQLSSLRSEDTAVYFCAKAATSGWSPFDNWGQGTLVTVSS

SEQ ID NO: 63 - DNA, 321 bp, synthetic construct
  source 1..321: mol_type = other DNA
gaaattgtgttgacgcagtctccaggcaccctgtctttgtctccaggggaaagagccaccctctcctgcagggccagtcagagtattagcagcagctactttggctggtaccagcagaaacctggccaggctcccaggctcctcatctatggtgcatccaggagggccgctggcatcccagacaggttcagttgcagtgggtctgggacagacttcactctcaccatcagcagactggagcctgaagattttggagtgtattactgtcagcagtatggtagctcaccgaccttcggccaagggaccaaggtggaaatcaaa

SEQ ID NO: 64 - protein, 107 aa, synthetic construct
EIVLTQSPGTLSLSPGERATLSCRASQSISSSYFGWYQQKPGQAPRLLIYGASRRAAGIPDRFSCSGSGTDFTLTISRLEPEDFGVYYCQQYGSSPTFGQGTKVEIK

SEQ ID NO: 65 - DNA, 393 bp, synthetic construct
  source 1..393: mol_type = other DNA
gaggtgcagctggcggagtctgggggaggcgtggtccagcctgggaggtccctgagactctcctgtgcagcgtctggattcaccttcaatagctatggcatgcactgggtccgccaggctccaggcaaggggctggagtgggtggcagtcatctgggatgatggaagtaaaaaatattatgcagactccgtgaagggccgattcaccatctccagagacaattccaagaacacgctgtttctgcaaatgaacagcctgagagccgaggacacggctgtgtattactgtgcgagagagacaattatgtttacatttggggaagttatcgttagcagtagggacaactactacatggacgtctggggcaaagggaccacggtcaccgtctcctca

SEQ ID NO: 66 - protein, 131 aa, synthetic construct
EVQLAESGGGVVQPGRSLRLSCAASGFTFNSYGMHWVRQAPGKGLEWVAVIWDDGSKKYYADSVKGRFTISRDNSKNTLFLQMNSLRAEDTAVYYCARETIMFTFGEVIVSSRDNYYMDVWGKGTTVTVSS

SEQ ID NO: 67 - DNA, 324 bp, synthetic construct
  source 1..324: mol_type = other DNA
gaaattgtgttgacacagtctccagccaccctgtctctgtctccaggggaaagagccaccctctcctgcagggccagtcagagtgttagcaactacttagcctggtaccaacagaaacctggccaggctcccaggctcctcatctatgatgcatctaccagggccattggcatcccagccaggttcagtggcgttgggtctgggacagacttcactctcaccatcagcagcctagagcctgaagattttgcagtttattactgtcagcagcgaagcaactggcctcctctcactttcggcggagggaccaaggtggagatcaaa

SEQ ID NO: 68 - protein, 108 aa, synthetic construct
EIVLTQSPATLSLSPGERATLSCRASQSVSNYLAWYQQKPGQAPRLLIYDASTRAIGIPARFSGVGSGTDFTLTISSLEPEDFAVYYCQQRSNWPPLTFGGGTKVEIK

SEQ ID NO: 69 - DNA, 366 bp, synthetic construct
  source 1..366: mol_type = other DNA
caggtccagctggtgcagtctggggctgaggtgaagaagcctgggtcctcggtgaaggtctcctgcaaggcctctggagacaccttcagcacctatgctatcacctgggtgcgacaggcccctggacaagggcttgagtggatgggaaggatcatccctatccttggtgtaacaaactatgcacagaagttccagggcagagtcacgtttaccgcggacaaatccacgatcacagcctacttggagctggtgaacctgagatctgaagacacggccgtatattactgtacgagagatgggtacggagggcgggacctacttttaaactactactggggccagggaaccctggtcaccgtctcctca

SEQ ID NO: 70 - protein, 122 aa, synthetic construct
QVQLVQSGAEVKKPGSSVKVSCKASGDTFSTYAITWVRQAPGQGLEWMGRIIPILGVTNYAQKFQGRVTFTADKSTITAYLELVNLRSEDTAVYYCTRDGYGGRDLLLNYYWGQGTLVTVSS

SEQ ID NO: 71 - DNA, 324 bp, synthetic construct
  source 1..324: mol_type = other DNA
gaaattgtgttgacgcagtctccaggcttgctgttttggtttccaggggaaagagccaccctctcctgcagggccagccagagtgtcagcagcagcaagttagcctggtaccaacagaaacctggccaggctcccaggctcctcatctatggtgcatccagcagggcccctggcatcccagacaggttcagtggcagtgggtctgggacagacttcactctcaccatcaatagactggagcctgaagattttgcagtgtatttctgtcagcagtatggcagcgctcagtggacgttcggccaagggaccaaggtggacttcaag

SEQ ID NO: 72 - protein, 108 aa, synthetic construct
EIVLTQSPGLLFWFPGERATLSCRASQSVSSSKLAWYQQKPGQAPRLLIYGASSRAPGIPDRFSGSGSGTDFTLTINRLEPEDFAVYFCQQYGSAQWTFGQGTKVDFK

SEQ ID NO: 73 - DNA, 387 bp, synthetic construct
  source 1..387: mol_type = other DNA
gaagtgcagctggtggagtctgggggaggcgtggtccagcctgggaggtccctgagactctcctgtgcagcgtctggattcaccttcagtcagtatggcatgcactgggtccgccaggctccaggcaaggggctggagtgcgtggcagttatatggtttgatggaagtaagaaatactatggagactccgtgaagggccgattcaccatctccagagacaattccaagaacacgctgtatctgcaaatgaacagcctgagagccgaggacacggctgtgtatttctgtgcgagagagagtctgtattactatgatagtggtggtcattactacgagagagggagaattgactactggggccagggaaccctggtcaccgtctcctca

SEQ ID NO: 74 - protein, 129 aa, synthetic construct
EVQLVESGGGVVQPGRSLRLSCAASGFTFSQYGMHWVRQAPGKGLECVAVIWFDGSKKYYGDSVKGRFTISRDNSKNTLYLQMNSLRAEDTAVYFCARESLYYYDSGGHYYERGRIDYWGQGTLVTVSS

SEQ ID NO: 75 - DNA, 321 bp, synthetic construct
  source 1..321: mol_type = other DNA
gacatccagatgacccagtctccatcctcactgtctgcatctgtgggagacagagtcaccatcacttgtcgggcgagtcaggacattagtaattttttagcctggtttcaacagaaaccagggaaagcccctaagtcccttatctatgctgcatccagtttgctaagtggggtcccatcaaagttcagcggcagtggatctgggacagatttcactctcaccatcagcagcctgcagcctgaagattttgcaacttattactgccaacagtataatagttaccctcccacttttggccaggggaccaagctggagatcaaa

SEQ ID NO: 76 - protein, 107 aa, synthetic construct
DIQMTQSPSSLSASVGDRVTITCRASQDISNFLAWFQQKPGKAPKSLIYAASSLLSGVPSKFSGSGSGTDFTLTISSLQPEDFATYYCQQYNSYPPTFGQGTKLEIK

SEQ ID NO: 77 - DNA, 390 bp, synthetic construct
  source 1..390: mol_type = other DNA
gaggtgcagctggtggagtctggggggggcgtggtccagcctgggaggtccctgagactctcctgtgcagcgtctggattcagtttcagtaactatgccatgcactgggtccgccaggctccaggcaagggactggagtgggtggcagttacatggtatgatggaagtagtaagtaccatgcagactccgtgaagggccgattcaccatctccagagacaattcgaagaacacgctgtatctgcaaatgaacagcctgagagccgaggacacggctgtctattactgtgcgagatgggggatgggtatagtagcctccccgggcaacgtactgaactactactactttatggacgtctggggccaagggaccacggtcatcgtctcctca

SEQ ID NO: 78 - protein, 130 aa, synthetic construct
EVQLVESGGGVVQPGRSLRLSCAASGFSFSNYAMHWVRQAPGKGLEWVAVTWYDGSSKYHADSVKGRFTISRDNSKNTLYLQMNSLRAEDTAVYYCARWGMGIVASPGNVLNYYYFMDVWGQGTTVIVSS

SEQ ID NO: 79 - DNA, 321 bp, synthetic construct
  source 1..321: mol_type = other DNA
tcctatgagctgacacagccaccctcggtgtcagtgtccccaggacagacggccaggatcacctgctctggaaatccattgccaaagaaacatacttactggtaccagcagaagccaggccaggcccctgtgttaatgatatataaagacagtgagaggccctcacggatccctgagcgattctccggctccagttcagggacaacagtcacgttgaccatcagtggagtccaggcagaagacgaggctgactattactgtcaatcagcagacagcactggtattgtggttttcggcggagggaccaagctgaccgtccta

SEQ ID NO: 80 - protein, 107 aa, synthetic construct
SYELTQPPSVSVSPGQTARITCSGNPLPKKHTYWYQQKPGQAPVLMIYKDSERPSRIPERFSGSSSGTTVTLTISGVQAEDEADYYCQSADSTGIVVFGGGTKLTVL

SEQ ID NO: 81 - DNA, 366 bp, synthetic construct
  source 1..366: mol_type = other DNA
caggtccagctggtgcagtctggggctgaggtgaagaagcctgggtcctcggtgaaggtctcctgcaaggcttctggaggcaccttcagcacctatgcaatcacctgggtgcgacaggcccctggacaagggcttgagtggatgggaaggatcatccctatccttcgagtagcaaactacgcacagaagttccagggcagagtcacgattaccgcggacagctccacgagcacagcctacatggagctgagcagcctgagatctgaggacacggccgtgtattactgtgcgaacctactagcatattgtggtggtgactgcagatctgactactggggccagggaaccctggtcaccgtctcctca

SEQ ID NO: 82 - protein, 122 aa, synthetic construct
QVQLVQSGAEVKKPGSSVKVSCKASGGTFSTYAITWVRQAPGQGLEWMGRIIPILRVANYAQKFQGRVTITADSSTSTAYMELSSLRSEDTAVYYCANLLAYCGGDCRSDYWGQGTLVTVSS

SEQ ID NO: 83 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttaagaacagctacttagcctggtaccaccagaaacctggccaggctcccaggctcctcatctatggtgcatccagcagggccactggcatcccagacaggttcagtggcagtgggtctgggacagacttcactctcaccatcagcagactggagcctgaagattttgcggtgtattactgtcagcagtacggtagctcactttggacgttcggccaagggaccaaggtggatgtcaaa

SEQ ID NO: 84 - protein, 108 aa, synthetic construct
EIVLTQSPGTLSLSPGERATLSCRASQSVKNSYLAWYHQKPGQAPRLLIYGASSRATGIPDRFSGSGSGTDFTLTISRLEPEDFAVYYCQQYGSSLWTFGQGTKVDVK

SEQ ID NO: 85 - DNA, 366 bp, synthetic construct
  source 1..366: mol_type = other DNA
caggtccagctggtgcagtctggggctgaggtgaagaagcctgggtcctcggtgaaggtctcctgcaaggcttctggaggcaccttcagcaactatggtatcagctgggtgcgacaggcccctggacaagggcttgagtggatgggaaggaacatccctatcattggtatagcaaactacgcacagaagttccaggacagagtcacgattaccgcggacaaatccacgagcacagtgttcatggagctgagaagcctgagatatgaggacacggccgtatattactgtgcgagagaccctctagcagctcgccggggaaactggttcgacccctggggccagggaaccctggtcaccgtctcctca

SEQ ID NO: 86 - protein, 122 aa, synthetic construct
QVQLVQSGAEVKKPGSSVKVSCKASGGTFSNYGISWVRQAPGQGLEWMGRNIPIIGIANYAQKFQDRVTITADKSTSTVFMELRSLRYEDTAVYYCARDPLAARRGNWFDPWGQGTLVTVSS

SEQ ID NO: 87 - DNA, 321 bp, synthetic construct
  source 1..321: mol_type = other DNA
gacatccagatgacccagtctccttccaccctgtctgcatctgtaggagacagagtcaccatcacttgccgggccagtcagagtgttagtagttggttggcctggtatcagcagaaaccagggaaagcccctaaactcctgatctatgatgcctccactttgcaaagtggggtcccatcaaggttcagcggcagtggatctgggacagaattcactctcaccatcagcagcctgcagcctgatgatttcgcaacttattactgccaacagtataatagttattcgtacacttttggccaggggaccaagctggagatcaaa

SEQ ID NO: 88 - protein, 107 aa, synthetic construct
DIQMTQSPSTLSASVGDRVTITCRASQSVSSWLAWYQQKPGKAPKLLIYDASTLQSGVPSRFSGSGSGTEFTLTISSLQPDDFATYYCQQYNSYSYTFGQGTKLEIK

SEQ ID NO: 89 - DNA, 393 bp, synthetic construct
  source 1..393: mol_type = other DNA
gaagtgcagctggtggagtctgggggaggcttggtacagcctggagggtccctgagactctcctgtgcagcctctggactcactttcagtgcttatgaaatgaactgggtccgccaggctccaggaaaggggctggagtgggtttcacacattactactagtggcagtgccatatactacgcagattctgtgaagggccgattcaccatctccagagacaacgccaagaactcactgcatctgcaaatgaacagactgagagccgacgacacggctgtatatttctgtgcgagagatagtaccatcgttcaatgtagtggcaccagctgccatgtgtccacctactcctacatggacgtctggggcaaagggaccacagtcaccgtctcttca

SEQ ID NO: 90 - protein, 131 aa, synthetic construct
EVQLVESGGGLVQPGGSLRLSCAASGLTFSAYEMNWVRQAPGKGLEWVSHITTSGSAIYYADSVKGRFTISRDNAKNSLHLQMNRLRADDTAVYFCARDSTIVQCSGTSCHVSTYSYMDVWGKGTTVTVSS

SEQ ID NO: 91 - DNA, 324 bp, synthetic construct
  source 1..324: mol_type = other DNA
gaaattgtgttgacacagtctccagccaccctgtctttgtctccaggggaaagagccaccctctcctgcagggccagtcagagtgttaccacctacttagcctggtaccaacacaaacctggccaggctcccaggctcctcatttatgatgcatccaagagggccactggcatcccagccaggttcagtggcagtgggtctgggacagacttcactctcaccatcagcagcctagagcctgaagattttgcagtttatcactgtcagcagcgtagcagctggcctccgatcaccttcggccaagggacacgcctggagattaaa

SEQ ID NO: 92 - protein, 108 aa, synthetic construct
EIVLTQSPATLSLSPGERATLSCRASQSVTTYLAWYQHKPGQAPRLLIYDASKRATGIPARFSGSGSGTDFTLTISSLEPEDFAVYHCQQRSSWPPITFGQGTRLEIK

SEQ ID NO: 93 - DNA, 357 bp, synthetic construct
  source 1..357: mol_type = other DNA
gaagtgcagctggtggagtctggaggaggcttgatccagcctggggggtccctgagactctcctgtgcagcctctgggttcaccgtcagtagccactacatgagttgggtccgccaggctccagggaaggggctggagtgggtctcaggtatttatagcggtggtaggacacactacgcagactccgtgaagggccgattcaccatctccagagacaattccaagaacacgctgtatcttcaaatgaacagcctgagagccgatgacacggccgtgtattcctgtgcgagagggaccctactttacttgtacggtatggacgtctggggccaagggaccacggtcaccgtctcctca

SEQ ID NO: 94 - protein, 119 aa, synthetic construct
EVQLVESGGGLIQPGGSLRLSCAASGFTVSSHYMSWVRQAPGKGLEWVSGIYSGGRTHYADSVKGRFTISRDNSKNTLYLQMNSLRADDTAVYSCARGTLLYLYGMDVWGQGTTVTVSS

SEQ ID NO: 95 - DNA, 330 bp, synthetic construct
  source 1..330: mol_type = other DNA
cagtctgtgctgacgcagccgccctcagtgtctgcggccccagggcagagggtcaccatctcctgcactgggagcagctccaacatcggggcaggttataatgtacactggtaccagcaacttccaggagcagcccccaaactcctcatctatggtaacaccaatcggccctcaggggtccctgaccgattctctggctccaagtctggcacctcagcctccctggccatcactgggctccaggctgaggatgaggctgattattactgccagtcctttgacagcagcctgagtggggtcttcggaactgggaccaaggtcaccgtcctg

SEQ ID NO: 96 - protein, 110 aa, synthetic construct
QSVLTQPPSVSAAPGQRVTISCTGSSSNIGAGYNVHWYQQLPGAAPKLLIYGNTNRPSGVPDRFSGSKSGTSASLAITGLQAEDEADYYCQSFDSSLSGVFGTGTKVTVL

SEQ ID NO: 97 - DNA, 393 bp, synthetic construct
  source 1..393: mol_type = other DNA
gaagtgcagctggtggagtctgggggaggcttggtacagcctggagggtccctgagactctcctgttcagcctctggattcaccttccacagttacgacatgaactgggcacgccaggctccagggaaggggctggagtgggtttcagagattagtagtagtgggcaaagcaaaaattacgcagactctgtgaggggccgattcaccatctccagagacaacgccaagaactcactgtatctgcaaataaacagcctgcgaagcgaggacacggctgtttattactgtgcgcgagtgggttttgtggccaagtttgggagtgatttttctatcactcacgggacgtactacttggactactggggccagggaaccctggtcaccgtctcctca

SEQ ID NO: 98 - protein, 131 aa, synthetic construct
EVQLVESGGGLVQPGGSLRLSCSASGFTFHSYDMNWARQAPGKGLEWVSEISSSGQSKNYADSVRGRFTISRDNAKNSLYLQINSLRSEDTAVYYCARVGFVAKFGSDFSITHGTYYLDYWGQGTLVTVSS

SEQ ID NO: 99 - DNA, 324 bp, synthetic construct
  source 1..324: mol_type = other DNA
caacttgtgttgacgcagtctccagccaccctgtctttgtctccaggggaaagagccaccctctcctgcagggccagtcagagtgttagcaactacttagcctggtaccaacagaaacctggccaggctcccaggctcctcatctatgatgcatccaacagggcccctggcgttccagccaggttcagtggcagtgggtctgggacagacttctctctcaccatcagcagccttgagcctgaagattttgcagtttattactgtctgcagcgtagcaactggcctccttggacgttcggccaagggaccaaggtggaaatcaaa

SEQ ID NO: 100 - protein, 108 aa, synthetic construct
QLVLTQSPATLSLSPGERATLSCRASQSVSNYLAWYQQKPGQAPRLLIYDASNRAPGVPARFSGSGSGTDFSLTISSLEPEDFAVYYCLQRSNWPPWTFGQGTKVEIK

SEQ ID NO: 101 - DNA, 384 bp, synthetic construct
  source 1..384: mol_type = other DNA
gaggtgcagctggtggagagcggcggcggcgtggtgcagcccggcaggagcctgaggctgagctgcgccgccagcggcttcaccttcagcagctacgagctgcactgggtgaggcaggcccccggcaagggcctggagtgggtggccgtgatcagctacgacggcagcagcaagtactacgccgacagcctgaagggcaggttcaccatcagcagggacaacagcaagaacaccctgagcctgcagatgaacagcctgagggccgaggacaccgccatgtactactgcgtgaggcagagcgaggcctactgcagcgacggcaggtgcgactggaggagggccctggacatctggggccagggcaccatggtgaccgtgagcccc

SEQ ID NO: 102 - protein, 128 aa, synthetic construct
EVQLVESGGGVVQPGRSLRLSCAASGFTFSSYELHWVRQAPGKGLEWVAVISYDGSSKYYADSLKGRFTISRDNSKNTLSLQMNSLRAEDTAMYYCVRQSEAYCSDGRCDWRRALDIWGQGTMVTVSP

SEQ ID NO: 103 - DNA, 324 bp, synthetic construct
  source 1..324: mol_type = other DNA
gacatccagatgacccagtctccatcctccctgtctgcatctgtaggagacagagtcaccatcacttgccaggcgagtcaggacattaagaactatttaaattggtatcagcagaaaccagggaaagcccctaagctcctgatctacgatgtatccaaattggaaacaggggtcccatcaaggttcagtggaagtggatctgggacagatttttctttcaccatcagcagcctgcagcctgaagatattgcaacatattactgtcaacaatatgataatctgccctcgtacacttttggccaggggaccaagctggagatcaaa

SEQ ID NO: 104 - protein, 108 aa, synthetic construct
DIQMTQSPSSLSASVGDRVTITCQASQDIKNYLNWYQQKPGKAPKLLIYDVSKLETGVPSRFSGSGSGTDFSFTISSLQPEDIATYYCQQYDNLPSYTFGQGTKLEIK

SEQ ID NO: 105 - DNA, 370 bp, synthetic construct
  source 1..370: mol_type = other DNA
gaggtgcagctggtggagagcggcggcggcctggtgcagcccggcggcagcctgaggctgagctgcgccgccagcggcttcaccttcagcagctacggcatccactgggtgaggcaggcccccggcaagggcctggagtgggtggccatcatcagctacgacggcaacaagaagttccacgccgacagcgtgaaggacaggttcaccatcagcagggacaacagcaagaacatgctgtacctgcagatgaacaacctgaagctggaggacaccgccgtgtactactgcgccagggcctacgacagcggctacttcgccggcagcgtgttctactactggggccagggcgtgctggtgaccgtgagcagcg

SEQ ID NO: 106 - protein, 123 aa, synthetic construct
EVQLVESGGGLVQPGGSLRLSCAASGFTFSSYGIHWVRQAPGKGLEWVAIISYDGNKKFHADSVKDRFTISRDNSKNMLYLQMNNLKLEDTAVYYCARAYDSGYFAGSVFYYWGQGVLVTVSS

SEQ ID NO: 107 - DNA, 334 bp, synthetic construct
  source 1..334: mol_type = other DNA
gaggtggtgttcacccagccccacagcgtgagcggcagccccggccagaccgtgaccatcagctgcaccaggagcagcggcagcatcgacagcgagtacgtgcagtggtaccagcagaggcccggcaacgcccccaccaccgtgatctacaaggacaaccagaggcccagcggcgtgcccgacaggttcagcggcagcatcgacagcagcagcaacagcgccagcctggccatcagcggcctgaagagcgaggacgaggccgactactactgccagagcgccgacggcaactaccaccccgtgttcggcgagggcaccaggctgaccgtgctgg

SEQ ID NO: 108 - protein, 111 aa, synthetic construct
EVVFTQPHSVSGSPGQTVTISCTRSSGSIDSEYVQWYQQRPGNAPTTVIYKDNQRPSGVPDRFSGSIDSSSNSASLAISGLKSEDEADYYCQSADGNYHPVFGEGTRLTVL

SEQ ID NO: 109 - DNA, 370 bp, synthetic construct
  source 1..370: mol_type = other DNA
gaggtgcagctggtggagagcggcggcggcgtggtgcagcccggcggcagcctgaggctgagctgcgccgccagcggcttcaccttcgacgactacgccatgcactgggtgaggcaggcccccggcaagggcctggagtgggtgagcggcatcagctggagcggcggcaacacctactacgccgacagcgtgaagggcaggttcaccgtgagcagggagaacgccaagaacagcctgtacctgcagatgaacaggctgagggccgaggacaccgccttctacaactgcgccaggaccagccagggcggcgccgtggtgttcagcgcccccaactactggggccagggcgtgctggtgaccgtgagcagcg

SEQ ID NO: 110 - protein, 123 aa, synthetic construct
EVQLVESGGGVVQPGGSLRLSCAASGFTFDDYAMHWVRQAPGKGLEWVSGISWSGGNTYYADSVKGRFTVSRENAKNSLYLQMNRLRAEDTAFYNCARTSQGGAVVFSAPNYWGQGVLVTVSS

SEQ ID NO: 111 - DNA, 331 bp, synthetic construct
  source 1..331: mol_type = other DNA
cagagcgtgctgacccaggcccccagcgtgagcggcgcccccggccagagggtgaccatcagctgcaccggcagcagcagcaacatcggcggctactacgtgcagtggtaccagcagctgcccggcagggcccccaagctgctgatctacgagaacaacaagaggcccagcggcgtgagcgacaggttcagcggcagcaggagcgccagcagcgccagcctgaccatcaccggcctgcagagcgaggacgaggccgactactactgccagagctacgacagcaggctgaacggctacatcttcggcggcggcaccaggctgaccgtgctgg

SEQ ID NO: 112 - protein, 110 aa, synthetic construct
QSVLTQAPSVSGAPGQRVTISCTGSSSNIGGYYVQWYQQLPGRAPKLLIYENNKRPSGVSDRFSGSRSASSASLTITGLQSEDEADYYCQSYDSRLNGYIFGGGTRLTVL

SEQ ID NO: 113 - DNA, 364 bp, synthetic construct
  source 1..364: mol_type = other DNA
gaggtgcagctggtggagagcggcggcggcctggtgcagcccggcggcagcctgaggctgagctgcgccgccagcgccttcaccttcgacgactacgccatgcactgggtgaggcaggcccccggcaagggcctggagtgggtgagcggcatcacctggagcggcctgagcacctactacgccgacagcgtgaagggcaggttcaccatcagcagggacaacgccaagaacagcctgtacctgcagatgaacagcctgagggtggaggacaccgccctgtactactgcgccaagagcggcaacaacagccccttcagcggcctggacagctggggccagggcgccgtggtgaccgtgagcagcg

SEQ ID NO: 114 - protein, 121 aa, synthetic construct
EVQLVESGGGLVQPGGSLRLSCAASAFTFDDYAMHWVRQAPGKGLEWVSGITWSGLSTYYADSVKGRFTISRDNAKNSLYLQMNSLRVEDTALYYCAKSGNNSPFSGLDSWGQGAVVTVSS

SEQ ID NO: 115 - DNA, 334 bp, synthetic construct
  source 1..334: mol_type = other DNA
cagagcgtgctgacccagccccccagcgtgagcggcgcccccggccagagggtgaccgtgagctgcaccggcagcagcagcaacatcggcgccggcagctacgtgcagtggtaccagcagctgcccggcaccgcccccaagctgctgatctacgagaacaacaagaggcccagcggcgtgagcgacaggttcagcggcagcaagagcggcaccagcgccagcctgaccatcaccggcctgcagagcgaggacgaggccgactactactgccagagctacgacagcagcctgaacgcctacatcttcggcgccggcaccaggctgaccgtgctgg

SEQ ID NO: 116 - protein, 111 aa, synthetic construct
QSVLTQPPSVSGAPGQRVTVSCTGSSSNIGAGSYVQWYQQLPGTAPKLLIYENNKRPSGVSDRFSGSKSGTSASLTITGLQSEDEADYYCQSYDSSLNAYIFGAGTRLTVL

SEQ ID NO: 117 - DNA, 355 bp, synthetic construct
  source 1..355: mol_type = other DNA
aagagccagctggtggagagcggcggcggcctggtgcagcccggcggcagcctgaggctgagctgcgccgccagcggcttcaccttcgacgcctacgccatgcactgggtgaggcaggcccccggcgagggcctggagtgggtgaccgccatcagctggagcggcagcagcacctactacgccgacagcgtgaagggcaggttcaccttcagcagggacaacgccaagaacagcctgtacctggagatgaacagcctgagggccgacgacaccgccttctactactgcgtgaagggcggcgtgtacaactggttcgacgtgtggggccccggcgtgctggtgaccgtgagcagcg

SEQ ID NO: 118 - protein, 118 aa, synthetic construct
KSQLVESGGGLVQPGGSLRLSCAASGFTFDAYAMHWVRQAPGEGLEWVTAISWSGSSTYYADSVKGRFTFSRDNAKNSLYLEMNSLRADDTAFYYCVKGGVYNWFDVWGPGVLVTVSS

SEQ ID NO: 119 - DNA, 331 bp, synthetic construct
  source 1..331: mol_type = other DNA
cagagcgtgctgacccagccccccagcgtgagcggcgcccccggccagagggtgaccatcagctgcaccggcagcaggagcaacatcggcggctactacgtgagctggtaccagcagttccccggcaccacccccaagctgctgatctaccaggacaacaagaggcccagcggcgtgagcgacaggttcagcggcagcaagagcggcaccagcgccagcctgaccatcaccggcctgcagaccgaggacgaggccgactactactgcctgagctacgacagcagcctgaacggctgggtgttcggcggcggcaccaggctgaccgtgctgg

SEQ ID NO: 120 - protein, 110 aa, synthetic construct
QSVLTQPPSVSGAPGQRVTISCTGSRSNIGGYYVSWYQQFPGTTPKLLIYQDNKRPSGVSDRFSGSKSGTSASLTITGLQTEDEADYYCLSYDSSLNGWVFGGGTRLTVL

SEQ ID NO: 121 - DNA, 367 bp, synthetic construct
  source 1..367: mol_type = other DNA
gagatgcagctggtggagagcggcggcggcctggtgcagcccggcggcagcctgaggctgagctgcgccgccagcggcttcaccttcgacgcctacgccatgcactgggtgaggcagaggcccggcaagggcctggagtggatcagcggcatcatctggagcggcaggaccacctactacgccgacagcgtgaagggcaggttcaccttcagcagggacaacagcaagaacagcctgtacctggagatgaacagcctgaagcccgaggacaccgccctgtactactgcgccaaggacagctggaactacggcgtgtacaacagcctggacgtgtggggcaggggcatcctggtgaccgtgagcagcg

SEQ ID NO: 122 - protein, 122 aa, synthetic construct
EMQLVESGGGLVQPGGSLRLSCAASGFTFDAYAMHWVRQRPGKGLEWISGIIWSGRTTYYADSVKGRFTFSRDNSKNSLYLEMNSLKPEDTALYYCAKDSWNYGVYNSLDVWGRGILVTVSS

SEQ ID NO: 123 - DNA, 331 bp, synthetic construct
  source 1..331: mol_type = other DNA
cagagcgtgctgacccagccccccagcgtgagcggcgcccccggccagagggtgaccatcagctgcaccggcagcagcagcaacgtgggcccccactacgtgagctggtaccagcacctgcccggcaccacccccaagctgctgatcttccaggacaacaagaggcccagcggcgtgagcgacaggttcagcggcagcaagagcgccagcagcgccagcctgaccatcaccggcctgcagaccgaggacgaggccgactactactgcctgagctacgacagcagcctgaacgtgtacatcttcggcagcggcaccaggctgaccgtgctgg

SEQ ID NO: 124 - protein, 110 aa, synthetic construct
QSVLTQPPSVSGAPGQRVTISCTGSSSNVGPHYVSWYQHLPGTTPKLLIFQDNKRPSGVSDRFSGSKSASSASLTITGLQTEDEADYYCLSYDSSLNVYIFGSGTRLTVL

SEQ ID NO: 125 - DNA, 367 bp, synthetic construct
  source 1..367: mol_type = other DNA
gaggagaggctggtggagagcggcggcggcctggtgcagcccggcggcagcctgaggctgagctgcgccgccagcggcttcaccttcggcgactacgccatgcactgggtgaggcaggtgcccggcaagagcctggagtggctgagcggcatcagctggagcggctacaccacctacagcgccgacagcgtgaagggcaggttcaccatcagcagggacaacgccaagaacagcctgtacctgcagatgaacaggctgaggcccgaggacaccggcctgtactactgcgtgaccctgatcggcgccatcggcatcaaggacagcctggacgtgtggggcaggggcgtgctggtgaccgtgagcagcg

SEQ ID NO: 126 - protein, 122 aa, synthetic construct; identical to SEQ ID NO: 2

SEQ ID NO: 127 - DNA, 331 bp, synthetic construct
  source 1..331: mol_type = other DNA
gaggtggtgttcacccagccccacagcgtgagcggcagccccggccagatggtgaccatcagctgcaccaggagcagcggcaccatcgacaacagctacgtgtactggcaccagcagaggcccggcagcgcccccaccaccgtgatctacaacgacgaccagaggcccagcggcgtgcccgacaggttcagcggcagcatcgacagcagcagcaacagcgccagcctgaccatcagcggcctgaagagcgaggacgaggccgactactactgccagagctacgacagcagcggctgggtgttcggcggcggcaccaggctgaccgtgctgg

SEQ ID NO: 128 - protein, 110 aa, synthetic construct
EVVFTQPHSVSGSPGQMVTISCTRSSGTIDNSYVYWHQQRPGSAPTTVIYNDDQRPSGVPDRFSGSIDSSSNSASLTISGLKSEDEADYYCQSYDSSGWVFGGGTRLTVL

SEQ ID NO: 129 - DNA, 364 bp, synthetic construct
  source 1..364: mol_type = other DNA
caggtgcagctgcaggagagcggccccggcctggtgaagcccagcgagaccctgagcctgacctgcaccgtgagcggcggcagcatcaccggcaactactggacctgggtgaggcagccccccggcaagggcctgcagtacatcggcaggttcgacgccggcggcaagacctactacaacccccccctgaagagcagggtgagcatcctgggcgacaagagcaagaaccaggtgagcctgaacctgaccagcgtgaccgtggccgacaccggcgtgtactactgcgccagggagcagtggggccagctgctgagcggcagcttcgacgtgtggggcaggggcgtgctggtgaccgtgagcagcg

SEQ ID NO: 130 - protein, 121 aa, synthetic construct
QVQLQESGPGLVKPSETLSLTCTVSGGSITGNYWTWVRQPPGKGLQYIGRFDAGGKTYYNPPLKSRVSILGDKSKNQVSLNLTSVTVADTGVYYCAREQWGQLLSGSFDVWGRGVLVTVSS

SEQ ID NO: 131 - DNA, 325 bp, synthetic construct
  source 1..325: mol_type = other DNA
agctacgagctgacccagccccccagcgtgagcgtgagccccggccagaccgccaggatcacctgcagcggcgacgccctgcccgactactacgcccactggtaccagcagaagagcggccaggcccccatgctggtgatctacaaggacaccgagaggcccagcggcatccccgagaggttcagcggcagcagcagcggcaccaccgtgagcctgaccatcagcggcgtgcaggccgaggacgaggccgactactactgccagagcggcgacagcagcggcaaccactgggtgttcggcggcggcaccaggctgaccgtgctgg

SEQ ID NO: 132 - protein, 108 aa, synthetic construct
SYELTQPPSVSVSPGQTARITCSGDALPDYYAHWYQQKSGQAPMLVIYKDTERPSGIPERFSGSSSGTTVSLTISGVQAEDEADYYCQSGDSSGNHWVFGGGTRLTVL

SEQ ID NO: 133 - DNA, 367 bp, synthetic construct
  source 1..367: mol_type = other DNA
aagaggggctggtggagcctgggcgaggcctggagcagcctgggcggcagcctgaggctgagctgcgccgccagcggcttcaacttcggcgactacgccatgcactgggtgaggcaggtgcccggcaagagcctggagtggctgagcggcatcagctggagcggctacaccacctacagcgccgacagcgtgaagggcaggttcaccatcagcagggacaacgccaagaacagcctgtacctgcagatgaacaggctgaggcccgaggacaccggcctgtactactgcgtgaccctgatcggcgccatcggcatcaaggacagcctggacgtgtggggcaggggcgtgctggtgaccgtgagcagcg

SEQ ID NO: 134 - protein, 122 aa, synthetic construct
KRGWWSLGEAWSSLGGSLRLSCAASGFNFGDYAMHWVRQVPGKSLEWLSGISWSGYTTYSADSVKGRFTISRDNAKNSLYLQMNRLRPEDTGLYYCVTLIGAIGIKDSLDVWGRGVLVTVSS

SEQ ID NO: 135 - DNA, 334 bp, synthetic construct
  source 1..334: mol_type = other DNA
cagagcgtgctgacccagccccccagcgtgagcggcgcccccggccagagcatcaccgtgagctgcaccggcagcagcagcaacatcggcgccggcaacttcgtgcagtggtacaggcacctgcccggcaccgcccccaagctgctgatctaccagaccgacaagaggcccagcggcctgagcgacaggctgagcggcagcaggagcggcagcagcgccagcctgaccatcaccggcctgcagagcgacgacgaggccgactactactgccaggcctacgacagccacagcaacacctggctgttcggcggcggcaccaggctgaccgtgctgg

SEQ ID NO: 136 - protein, 111 aa, synthetic construct
QSVLTQPPSVSGAPGQSITVSCTGSSSNIGAGNFVQWYRHLPGTAPKLLIYQTDKRPSGLSDRLSGSRSGSSASLTITGLQSDDEADYYCQAYDSHSNTWLFGGGTRLTVL

SEQ ID NO: 137 - protein, 474 aa, synthetic construct
MGWSCIILFLVATATGVHSEERLVESGGGLVQPGGSLRLSCAASGFTFGDYAMHWVRQVPGKSLEWLSGISWSGYTTYSADSVKGRFTISRDNAKNSLYLQMNRLRPEDTGLYYCVTLIGAIGIKDSLDVWGRGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 138 - protein, 236 aa, synthetic construct
MGWSCIILFLVATATGVHSQSVLTQPPSVSGAPGQRVTVSCSGSSSNIGAGNYVQWYQQLPGTAPKVLIYQTEKRPSGTSDRFSGSKSDTSASLTINGLQSEDEADYYCQVYDSNLNGWVFGGGTRLTVLGQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 139 - protein, 470 aa, synthetic construct
MGWSCIILFLVATATGVHSQVQLQESGPGLVKPSQTLSLTCTVSGGSISRAGSYWSWIRQHPGKGLEWIGYISYSGSTYYNPSLESRVTMSLDTSKSQFSLKLSSVTAADTAVYYCAREDRSGFYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 240 aa, synthetic construct
MGWSCIILFLVATATGSVTQSVLTQPSSLSASPGATASLTCTLRSGINVDTYRIYWYQQKPGSPPQYLLRYKSDSDKHQGSGVPSRFSGSKDASANAGILLISGLQSADEADYYCMIWHSGAWVFGGGTKLTVQGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 141 - protein, 473 aa, synthetic construct
MGWSCIILFLVATATGVHSQVQLVQSGAELKKPGASVKVSCKASGYTFSDHYVHWVRQAPGQGLEWMGRINPNSGGTNYAQRFLGRVTMTRDASISTAYLDLSSLRSDDTAVYYCARSSSSWSGHYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 235 aa, synthetic construct
MGWSCIILFLVATATGSVTQSVLTQPPSVSAAPGQRVTISCSGSTSNIASNFVSWYQQLPGTAPKLLIYDNHKRPSGIPDRFSGSKSGTSATLGITGLQTGDEADYYCGTWDSSLSAGVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 143 - protein, 474 aa, synthetic construct
MGWSCIILFLVATATGVHSQVQLQESGPGLVKPSQTLSLTCTVSGGSISSGGYHWSWIRQHPGKGLEWIGNIYYNGRTYYNPSLKSRVTISVDTSENEFSLKLSSVTAADTAVYYCARDRRRPRIGHDYGMDVWGQGTKVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 234 aa, synthetic construct
MGWSCIILFLVATATGVHSDIQMTQSPSSLSASVGDRVTISCRASQSISNFLNWYQQKPGQAPKLLIYTASNLQSGVPSRFSGSGSGTDFTLTISSLQPEDFATYYCQQIYNTGMYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 19 aa, synthetic construct
MGWSCIILFLVATATGVHS

SEQ ID NO: 146 - protein, 19 aa, synthetic construct
MGWSCIILFLVATATGSVT

SEQ ID NO: 147 - protein, 333 aa, Macaca mulatta
ASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 148 - protein, 106 aa, Macaca mulatta
GQPKAAPSVTLFPPSSEELQANKATLVCLISDFYPGAVEVAWKADGSAVNAGVETTKPSKQSNNKYAASSYLSLTSDQWKSHKSYSCQVTHEGSTVEKTVAPAECS

SEQ ID NO: 149 - protein, 330 aa, Homo sapiens [sequence omitted]

SEQ ID NO: 150 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 151 - protein, 107 aa, Homo sapiens [sequence omitted]

SEQ ID NO: 152 - protein, 473 aa, synthetic construct
MGWSCIILFLVATATGVHSQVQLVQSGAELKKPGASVKVSCKASGYTFSDHYVHWVRQAPGQGLEWMGRINPNSGGTNYAQRFLGRVTMTRDASISTAYLDLSSLRSDDTAVYYCARSSSSWSGHYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 153 - protein, 235 aa, synthetic construct
MGWSCIILFLVATATGVHSQSVLTQPPSVSAAPGQRVTISCSGSTSNIASNFVSWYQQLPGTAPKLLIYDNHKRPSGIPDRFSGSKSGTSATLGITGLQTGDEADYYCGTWDSSLSAGVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 154 - protein, 472 aa, synthetic construct
MGWSCIILFLVATATGVHSQVQLQESGPGLVKPSQTLSLTCTVSGGSISRAGSYWSWIRQHPGKGLEWIGYISYSGSTYYNPSLESRVTMSLDTSKSQFSLKLSSVTAADTAVYYCAREDRSGFYGLDVWGQGTTVTVSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 155 - protein, 239 aa, synthetic construct
MGWSCIILFLVATATGVHSQSVLTQPSSLSASPGATASLTCTLRSGINVDTYRIYWYQQKPGSPPQYLLRYKSDSDKHQGSGVPSRFSGSKDASANAGILLISGLQSADEADYYCMIWHSGAWVFGGGTKLTVQSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 156 - protein, 470 aa, synthetic construct
MGWSCIILFLVATATGVHSQVQLQESGPGLVKPSQTLSLTCTVSGGSISRAGSYWSWIRQHPGKGLEWIGYISYSGSTYYNPSLESRVTMSLDTSKSQFSLKLSSVTAADTAVYYCAREDRSGFYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 157 - protein, 240 aa, synthetic construct
MGWSCIILFLVATATGVHSQSVLTQPSSLSASPGATASLTCTLRSGINVDTYRIYWYQQKPGSPPQYLLRYKSDSDKHQGSGVPSRFSGSKDASANAGILLISGLQSADEADYYCMIWHSGAWVFGGGTKLTVQGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 158 - protein, 474 aa, synthetic construct
MGWSCIILFLVATATGVHSQVQLVQSGAELKKPGASVKVSCKASGYTFSDHYVHWVRQAPGQGLEWMGRINPNSGGTNYAQRFLGRVTMTRDASISTAYLDLSSLRSDDTAVYYCARSSSSWSGHYYYYMDVWGKGTTVTVSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 159 - protein, 234 aa, synthetic construct
MGWSCIILFLVATATGVHSQSVLTQPPSVSAAPGQRVTISCSGSTSNIASNFVSWYQQLPGTAPKLLIYDNHKRPSGIPDRFSGSKSGTSATLGITGLQTGDEADYYCGTWDSSLSAGVFGGGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC